(19) United States
(12) Patent Application Publication
(10) Pub. No.: US 2003/0140372 A1
(43) Pub. Date: Jul. 24, 2003

(54) Genes for desaturases to alter lipid profiles in corn

(76) Inventor: Jennie Bih-Jien Shen, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/223,646

(22) Filed: Aug. 19, 2002

Related U.S. Application Data
(62) Division of application No. 09/326,285, filed on Jun. 7, 1999.

(51) Int. Cl. A01H 5/00; C12N 15/82; C07H 21/04; C12N 9/04; C12P 21/02; C12N 5/04

(52) U.S. Cl. 800/281; 800/320.1; 536/23.2; 435/190; 435/419; 435/69.1; 435/320.1

(57) ABSTRACT

The preparation and use of nucleic acid fragments comprising all or substantially all of a corn oleosin promoter, a stearoyl-ACP desaturase and a delta-12 desaturase which can be used individually or in combination to modify the lipid profile of corn are described. Chimeric genes incorporating such nucleic acid fragments and suitable regulatory sequences can be used to create transgenic corn plants having altered lipid profiles.

### FIELD OF THE INVENTION

The invention relates to the preparation and use of nucleic acid fragments comprising all or substantially all of a corn oleosin promoter, a stearoyl-ACP desaturase and a delta-12 desaturase which can be used individually or in combination to modify the lipid profile of corn. Chimeric genes comprising such nucleic acid fragments and suitable regulatory sequences can be used to create transgenic corn plants having altered lipid profiles.

### BACKGROUND OF THE INVENTION

Plant lipids have a variety of industrial and nutritional uses and are central to plant membrane function and climatic adaptation. These lipids represent a vast array of chemical structures, and these structures determine the physiological and industrial properties of the lipid. Many of these structures result either directly or indirectly from metabolic processes that alter the degree of unsaturation of the lipid. Different metabolic regimes in different plants produce these altered lipids, and either domestication of exotic plant species or modification of agronomically adapted species is usually required to produce economically large amounts of the desired lipid.

Plant lipids find their major use as edible oils in the form of triacylglycerols. The specific performance and health attributes of edible oils are determined largely by their fatty acid composition. Most vegetable oils derived from commercial plant varieties are composed primarily of palmitic (16:0), stearic (18:0), oleic (18:1), linoleic (18:2) and linolenic (18:3) acids. Palmitic and stearic acids are, respectively, 16- and 18-carbon-long, saturated fatty acids. Oleic, linoleic, and linolenic acids are 18-carbon-long, unsaturated fatty acids containing one, two, and three double bonds, respectively. Oleic acid is referred to as a mono-unsaturated fatty acid, while linoleic and linolenic acids are referred to as poly-unsaturated fatty acids. The relative amounts of saturated and unsaturated fatty acids in commonly used, edible vegetable oils are summarized below (Table 1):

TABLE 1
Percentages of Saturated and Unsaturated Fatty
Acids in the Oils of Selected Oil Crops

| | | Mono- | Poly |
|---|---|---|---|
| | Saturated | unsaturated | unsaturated |
| Canola | 6% | 58% | 36% |
| Soybean | 15% | 24% | 61% |
| Corn | 13% | 25% | 62% |
| Peanut | 18% | 48% | 34% |
| Safflower | 9% | 13% | 78% |
| Sunflower | 9% | 41% | 51% |
| Cotton | 30% | 19% | 51% |

Corn oil is comprised primarily of even-numbered carbon chain fatty acids. The distribution of fatty acids in typical corn oil is approximately 12% palmitic acid (16:0), 2% stearic acid (18:0), 25% oleic acid (18:1), 60% linoleic acid (18:2), and 1% linolenic acid (18:3). Palmitic and stearic acids are referred to as saturated fatty acids because their carbon chains contains only single bonds and the carbon chain is “saturated” with hydrogen atoms. Oleic, linoleic, and linolenic acids contain one, two, and three double bonds respectively, and are referred to as unsaturated fatty acids. Fatty acids in corn oil nearly always occur esterified to the hydroxyl groups of glycerol, thus forming triglycerides. Approximately 99% of refined corn oil is made up of triglycerides (“Corn Oil”, Corn Refiners Association, Inc., 1001 Connecticut Ave., N.W., Washington, D.C. 20036, 1986, 24 pp.).

Many recent research efforts have examined the role that saturated and unsaturated fatty acids play in reducing the risk of coronary heart disease. In the past, it was believed that mono-unsaturates, in contrast to saturates and poly-unsaturates, had no effect on serum cholesterol and coronary heart disease risk. Several recent human clinical studies suggest that diets high in mono-unsaturated fat and low in saturated fat may reduce the “bad” (low-density lipoprotein) cholesterol while maintaining the “good” (high-density lipoprotein) cholesterol (Mattson et al. (1985) Journal ofLipid Research 26:194-202).

A vegetable oil low in total saturates and high in mono-unsaturates would provide significant health benefits to consumers as well as economic benefits to oil processors. As an example, canola oil is considered a very healthy oil. However, in use, the high level of poly-unsaturated fatty acids in canola oil renders the oil unstable, easily oxidized, and susceptible to development of disagreeable odors and flavors (Gailliard (1980) in The Biochemistry of Plants Vol. 4, pp. 85-116, Stumpf, P. K., ea., Academic Press, New York). The levels of poly-unsaturates may be reduced by hydrogenation, but the expense of this process and the concomitant production of nutritionally questionable trans isomers of the remaining unsaturated fatty acids reduces the overall desirability of the hydrogenated oil (Mensink et al. (1990) N. Eng J. Med.N323: 439-445).

When exposed to air, unsaturated fatty acids are subject to oxidation which causes the oil to have a rancid odor. Oxidation is accelerated by high temperatures, such as in frying conditions. The rate of oxidation is enhanced in the cases of oils containing greater degrees of unsaturation. Thus, linoleic acid with two double bonds is more unstable than oleic acid which has only one double bond. Oxidation reduces the shelf life of products containing corn oil because of that oil's high proportion of linoleic acid. Corn oil and products containing corn oil are often packaged under nitrogen in special packaging materials such as plastic or laminated foil, or are stored under refrigeration to extend their shelf life. These extra measures to reduce oxidation and subsequent rancidity add considerable cost to products containing corn oil.

Another measure to reduce the effects of oxidation on corn oil is to chemically hydrogenate the oil. This commercially important process by which hydrogen is added to double bonds of unsaturated fatty acids changes the physical properties of the oil and extends the shelf life of products containing corn oil. Hydrogenated vegetable oils are used to make margarine, salad dressings, cooking oils, and shortenings, for example. Approximately half a billion pounds, or roughly 40-50% of corn oil produced in the U.S. is used for cooking and for salad oils (Fitch, B., (1985) JAOCS, Vol. 62, no. 11, pp. 1524-31). Production of a more stable oil by genetic means would clearly have value by reducing or eliminating the time and input costs of chemical hydrogenation.

In addition to the economic factors associated with chemical hydrogenation of corn oil, there are human health factors that favor the production of a natural high oleic oil. During the hydrogenation process, double bonds in fatty acids are completely hydrogenated or are converted from the cis configuration to the trans configuration. C is double bonds cause a fatty acid molecule to “bend,” which impairs crystallization and keeps the oil liquid at room temperature. During hydrogenation, cis bonds are straightened into the trans configuration, causing the oil to harden at room temperature. Recent studies on the effect of dietary trans fatty acids on cholesterol levels show that the trans isomer of oleic acid raises blood cholesterol levels at least as much as saturated fatty acids, which have been know for some time to raise cholesterol in humans (Mensink, R. P. and B. K. Katan, (1990) N. Engl. J. Med., 323:439-45). Furthermore, these studies show that the undesirable low density lipoprotein level increases and the desirable high density lipoprotein level decreases in response to diets high in trans fatty acids. Large amounts of trans fatty acids are found in margarines, shortenings, and oils used for frying; the most abundant trans fatty acid in the human diet is the trans isomer of oleic acid, elaidic acid.

While oils with low levels of saturated fatty acids are desirable from the standpoint of providing a healthy diet, fats that are solid at room temperature are required in some foods because of their functional properties. Such applications include the production of non-dairy margarines and spreads, and various applications in confections and in baking. Many animal and dairy fats provide the necessary physical properties, but they also contain both cholesterol and cholesterogenic medium-chain fatty acids. An ideal triglyceride for solid fat applications should contain a predominance of the very high melting, long chain fatty acid, stearic acid, and a balance of mono-unsaturated fatty acid with very little polyunsaturated fat. Natural plant solid fat fractions typically have a triacylglyceride structure with saturated fatty acids occupying the sn-1 and sn-3 positions of the triglycerides and an unsaturated fatty acid at the sn-2 position. This overall fatty acid composition and triglyceride structure confers an optimal solid fat crystal structure and a maximum melting point with minimal saturated fatty acid content.

The natural fat prototype for this high melting temperature vegetable fat is cocoa butter. More than 2 billion pounds of cocoa butter, the most expensive commodity edible oil, are produced worldwide. The U.S. imports several hundred million dollars worth of cocoa butter annually. High and volatile prices together with the uncertain supply of cocoa butter have encouraged the development of cocoa butter substitutes. The fatty acid composition of cocoa butter is 26% palmitic, 34% stearic, 35% oleic and 3% linoleic acids. About 72% of cocoa ‘butter’s triglycerides have the structure in which saturated fatty acids occupy positions 1 and 3 and oleic acid occupies position 2. Cocoa ‘butter’s unique fatty acid composition and distribution on the triglyceride molecule confer on it properties eminently suitable for confectionery end-uses: it is brittle below 27° C. and depending on its crystalline state, melts sharply at 25°-30° C. or 35°-36° C. Consequently, it is hard and non-greasy at ordinary temperatures and melts very sharply in the mouth. It is also extremely resistant to rancidity. For these reasons, producing corn oil with increased levels of stearic acid, especially in corn lines containing higher-than-normal levels of palmitic acid, and reduced levels of unsaturated fatty acids is expected to produce a cocoa butter substitute in com. This will provide additional value to oil and food processors as well as reduce the foreign import of certain tropical oils.

The human diet could also be improved by reducing saturated fat intake. Much of the saturated fat in the human diet comes from meat products. Poultry and swine diets often contain animal fat, which is high in saturated fatty acids, as an energy source. Non-ruminant animals such as these are very susceptible to tissue fatty acid alteration through dietary modification (M. F. Miller, et al. (1990) J. Anim. Sci., 68:1624-31). A large portion of animal feed rations is made up of com, which typically contains only about 4% oil. By replacing some or all of the supplemental animal fat in a feed ration with the oil present in high oil corn varieties, which contain up to 10% oil, it will be possible to produce meat products having a lower content of saturated fats. Feeding trials in which swine were fed diets high in oleic acid show that the amount of oleic acid deposited in adipose tissue can be raised substantially without adversely influencing the quality of the meat (M. F. Miller, et al.; L. C. St. John et al. (1987) J. Anim. Sci., 64: 1441-47). The degree of saturation of the fatty acids comprising an oil determines whether it is liquid or solid. In these studies, the animal diets high in oleic acid led to meat quality that was acceptable to the meat processing industry because of the low level of polyunsaturated fatty acids.

Only recently have serious efforts been made to improve the quality of corn oil through plant breeding, especially following mutagenesis, and a wide range of fatty acid composition has been discovered in experimental lines. These findings (as well as those with other oilcrops) suggest that the fatty acid composition of corn oil can be significantly modified without affecting the agronomic performance of a corn plant.

There are serious limitations to using mutagenesis to alter fatty acid composition. It is unlikely to discover mutations that a) result in a dominant (“gain-of-function”) phenotype, b) are in genes that are essential for plant growth, and c) are in an enzyme that is not rate-limiting and that is encoded by more than one gene. Even when some of the desired mutations are available in mutant corn lines, their introgression into elite lines by traditional breeding techniques will be slow and expensive, since the desired oil compositions in corn are most likely to involve several recessive genes.

Recent molecular and cellular biology techniques offer the potential for overcoming some of the limitations of the mutagenesis approach, including the need for extensive breeding. Some of the particularly useful technologies are seed-specific expression of foreign genes in transgenic plants [see Goldberg et al:(1989) Cell 56:149-160], and the use of antisense RNA to inhibit plant target genes in a dominant and tissue-specific manner [see van der Krol et al. (1988) Gene 72:45-50]. Other advances include the transfer of foreign genes into elite commercial varieties of commercial oilcrops, such as soybean [Chee et al. (1989) Plant Physiol. 91:1212-1218; Christou et al. (1989) Proc. Natl. Acad. Sci. U.S.A. 86:7500-7504; Hinchee et al. (1988) Bio/Technology 6:915-922; EPO publication 0 301 749 A2], rapeseed [De Block et al. (1989) Plant Physiol. 91:694-701], and sunflower [Everett et al.(1987) Bio/Technology 5:1201-1204], and the use of genes as restriction fragment length polymorphism (RFLP) markers in a breeding program, which makes introgression of recessive traits into elite lines rapid and less expensive [Tanksley et al. (1989) Bio/Technology 7:257-264]. However, application of each of these technologies requires identification and isolation of commercially-important genes.

WO 91/13972, published Sep. 19, 1991, describes desaturase enzymes relevant to fatty acid synthesis in plants, especially delta-9 desaturases.

U.S. Pat. No. 5,443,974, issued to Hitz et al. on Aug. 22, 1995, describes the preparation and use of nucleic acid fragments encoding soybean seed stearoyl-ACP desaturase enzymes or its precursor to modify plant oil composition.

WO 94/11516, published May 26, 1994, describes genes for microsomal delta-12 desaturases and related enzymes from plants. The cloning of a corn (Zea mays) cDNA encoding seed microsomal delta-12 fatty acid desaturase is described. The discussion of that citation is hereby incorporated by reference.

Oil biosynthesis in plants has been fairly well-studied [see Harwood (1989) in Critical Reviews in Plant Sciences Vol. 8(1): 1-43]. The biosynthesis of palmitic, stearic and oleic acids occur in the plastics by the interplay of three key enzymes of the “ACP track”: palmitoyl-ACP elongase, stearoyl-ACP desaturase and acyl-ACP thioesterase. Stearoyl-ACP desaturase introduces the first double bond on stearoyl-ACP to form oleoyl-ACP. It is pivotal in determining the degree of unsaturation in vegetable oils. Because of its key position in fatty acid biosynthesis it is expected to be an important regulatory step. While the ‘enzyme’s natural substrate is stearoyl-ACP, it has been shown that it can, like its counterpart in yeast and mammalian cells, desaturate stearoyl-CoA, albeit poorly [McKeon et al. (1982) J. Biol. Chem. 257:12141-12147]. The fatty acids synthesized in the plastic are exported as acyl-CoA to the cytoplasm. At least three different glycerol acylating enzymes (glycerol-3-P acyltransferase, 1-acyl-glycerol-3-P acyltransferase and diacylglycerol acyltransferase) incorporate the acyl moieties from the cytoplasm into triglycerides during oil biosynthesis. These acyltransferases show a strong, but not absolute, preference for incorporating saturated fatty acids at the sn-1 and sn-3 positions and monounsaturated fatty acid at the sn-2 of the triglyceride. Thus, altering the fatty acid composition of the acyl pool will drive a corresponding change in the fatty acid composition of the oil due to the effescts of mass action. Furthermore, there is experimental evidence that, because of this specificity, and given the correct composition of fatty acids, plants can produce oils suitable as cocoa butter substitutes [Bafor et al. (1990) JAOCS 67:217-225].

Based on the above discussion, one approach to altering the levels of stearic and oleic acids in vegetable oils is by altering their levels in the cytoplasmic acyl-CoA pool used for oil biosynthesis. There are two ways of doing this genetically. One of these ways is to alter the biosynthesis of stearic and oleic acids in the plastic by modulating the levels of stearoyl-ACP desaturase in seeds through either overexpression or antisense inhibition of its gene. Another is converting stearoyl-CoA to oleoyl-CoA in the cytoplasm through the expression of the stearoyl-ACP desaturase in the cytoplasm.

In order to use antisense or sense inhibition of stearoyl-ACP desaturase in the seed, it is essential to isolate the gene(s) or cDNA(s) encoding the target enzymes) in the seed, since either of these mechanisms of inhibition requires a high-degree of complementarily between the antisense RNA (see Stam et al. (1997) Annals of Botany 79:3-12) and the target gene. Such high levels of sequence complementarily or identity is not expected in stearoyl-ACP desaturase genes from heterologous species.

The purification and nucleotide sequences of mammalian microsomal stearoyl-CoA desaturases have been published [Thiede et al. (1986) J. Biol. Chem. 262:13230-13235; Ntambi et al. (1988) J. Biol. Chem. 263:17291-17300 and Kaestner et al. (1989) J. Biol. Chem. 264:14755-14761]. However, the plant enzyme differs from them in being soluble, in utilizing a different electron donor, and in its substrate-specificities. The purification and the nucleotide sequences for animal enzymes do not teach how to purify a plant enzyme or isolate a plant gene. The purification of stearoyl-ACP desaturase was reported from safflower seeds [McKeon et al. (1982) J. Biol. Chem. 257:12141-12147] and from soybean (U.S. Pat. No. 5,443,974).

The rat liver stearoyl-CoA desaturase protein has been expressed in E. coli [Strittmatter et al. (1988) J. Biol. Chem. 263:2532-2535] but, as mentioned above, its substrate specificity and electron donors are quite distinct from that of the plant.

Plant stearoyl-ACP desaturase cDNAs have been cloned from numerous species including safflower [Thompson et al. (1991) Proc. Natl. Acad. Sci. 88:2578], castor [Shanklin and Somerville (1991) Proc. Natl. Acad. Sci. 88:2510-2514], and cucumber [Shanklin et al. (1991) Plant Physiol. 97:467-468]. Kutzon et al. [(1992) Proc. Natl. Acad. Sci. 89:2624-2648] have reported that rapeseed stearoyl-ACP desaturase when expressed in Brassica rapa and B. napa in an antisense orientation can result in increase in 18:0 level in transgenic seeds.

Manipulation of stearate levels has been described (Knutzon, D. S. et al., (1992) Proc. Natl. Acad. Sci. USA 89(7): 2624-2628). It is possible to elevate the level of stearate seed oils by underexpression of stearoyl-ACP desaturase, the enzyme responsible for introducing the first double bond into 18 carbon fatty acids in plants. Seeds from both B. campestris and B. napus plants produced by antisense expression of a cDNA encoding the B. campestris stearoyl-ACP desaturase using a seed specific promoter region produced oils high in stearic acid, but also contained elevated levels of linolenic acid (18:3) when compared to unmodified plants from the same species. Elevated levels of stearic acid have been obtained in soybean by a similar underexpression of stearoyl-ACP desaturase (U.S. Pat. No. 5,443,974) and in canola by overexpression of an acyl-ACP thioesterase (U.S. Pat. No. 5,530,186). Mutation breeding has also produced soybean lines with elevated levels of stearic acid in their seed oils (Graef, G. L. et al., (1985) JAOCS 62:773-775; Hammond, E. G. and W. R. Fehr, (1983) Crop Sci. 23:192-193).

Poly-unsaturated fatty acids contribute to the low melting point of liquid vegetable oils. In high saturate oils their presence is a detriment in that they decrease melting point, and therefore even higher levels of undesirable saturated fatty acid are required to achieve a plastic fat at room temperature. Additionally, when used in baking and confectionery applications, high levels of poly-unsaturates leads to oxidative instability as described above for liquid oils. Thus for maximum utility a high saturate fat produced in corn should contain saturated fatty acids, mono-unsaturated fatty acid and as little poly-unsaturated fatty acid as possible. Gene combinations discovered in this invention provide novel fatty acid profiles in corn which meet these criteria. Other combinations result in a lipid profile in which the oleic acid content is not less than 60% of the total oil content. Many of these combinations also utilize a novel corn oleosin promoter or an intron/exon region from the shrunken 1 gene, or both an oleosin promoter and an intron/exon region from the shrunken 1 gene.

Lipid reserves in corn seeds are synthesized and stored primarily in a specialized tissue of the embryo called the scutellum. These lipid reserves constitute up to 50% of the dry weight of the embryo at seed maturity. As in all seeds, the storage lipid in corn seeds is packaged into simple organelles called oil bodies. These small spherical organelles consist of a triacylglycerol core surrounded by a single layer of phospholipids embedded with proteins termed oleosins (Huang(1985) Modern Methods of Plant Analysis 1:175-214; Stymme and Stobart (1987) The Biochemistry of Plants 10: 175-214; Yatsue and Jacks (1972) Plant Physiol. 49: 937-943; and Gurr (1980) The Biochemistry of plants 4: 205-248).

At least two classes of oleosin isoforms have been identified in diverse species of plants (Tzen et al. (1990) Plant Physiol. 94: 1282-1289). These two classes are arbitrarily named as high (H) and low (L) molecular weight isoforms within a particular species. Members of one isoform from diverse species are understood to be structurally related based on demonstrations of shared immunochemical properties and possession of significant amino acid sequence identity, and they are clearly distinct from members of the other isoform (Hatzopoulos et al. (1990) Plant Cell 2: 457-467; Lee and Huang (1994) Plant Mol. Biol. 26(6): 1981-1987; Murphy et al. (1991) Biochim. Biophys. Acta, 1088: 86-94; Qu and Huang (1990) J. Biol. Chem. 265: 2238-2243).

There are three oleosin isoforms present in corn seeds. They are found in the approximately proportional amounts of 2:1:1. These isoforms are named OLE16, OLE 17, and OLE 18, corresponding to their apparent molecular weights which range from approximately 16 kDa to 18 kDa. OLE17 and OLE18 are closely related members of the H class, whereas OLE16 is a member of the L class (Lee and Huang, 1994). The genes encoding the three oleosins have been cloned and sequenced (Qu and Huang (1990) J. Biol. Chem. 265: 2238-2243; and Huang, personal communication). The genes are expressed only in tissues within the embryo (scutellum and embryonic axis) and the aleurone layer during seed development, and are positively regulated by the hormone abscissic acid (Vance and Huang (1988) J. Biol. Chem. 263: 1476-1491; Huang (1992) Annul Rev. Plant Physiol. 35Plant Mol. Biol. 43: 177-200). The oleosins are highly expressed in the embryo, representing about 5-10% of the total scutellum protein or 2-8% of the total seed proteins.

Promoters from genes that display an embryo- and aleurone-specific (“embryo/aleurone”) pattern of expression, such as the oleosin genes, would be attractive candidates for use in transgenic approaches to direct the expression of a gene encoding an oil-modifying enzyme (Qu and Huang (1990) J. Biol. Chem. 265: 2238-2243; and Huang (1992)) or other enzymes of interest for embryo-specific traits, especially in corn. Another potential candidate gene from which to isolate a corn embryo/aleurone-specific promoter is the maize globulin-1 gene (Belanger and Kriz, 1989, Plant Physiol. 91: 636-643). However, to date, there is no report that describes the expression, regulation, or use of such promoters in either transient expression assays or stably integrated transgenic corn plants.

### SUMMARY OF THE INVENTION

This invention relates to an isolated nucleic acid fragment comprising a corn oleosin promoter wherein said promoter can be full length or partial and further wherein said promoter comprises a nucleotide sequence corresponding substantially to the nucleotide sequence in any of SEQ ID NOS:19 or 38-49 or said promoter comprises a fragment or subfragment that is substantially similar and functionally equivalent to any of the nucleotide sequences set forth in SEQ ID NOS:19 or 38-49.

In a second embodiment this invention concerns an isolated nucleic acid fragment encoding a corn delta-9 stearoyl-ACP desaturase corresponding substantially to a nucleotide sequence set forth in any of SEQ ID NOS:8 and 10 or any functionally equivalent subfragment thereof. Also included are chimeric genes comprising such fragments or subfragments thereof or the reverse complement of such fragment or subfragment which are operably linked to suitable regulatory sequences wherein expression of the chimeric gene results in an altered corn stearic acid phenotype.

In a third embodiment, this invention concerns an isolated nucleic acid fragment encoding a corn delta-12 desaturase corresponding substantially to the nucleotide sequence set forth in SEQ ID NO:2 or any functionally equivalent subfragment thereof as well as chimeric genes comprising such fragments or subfragments or the reverse complement of such fragment or subfragment which are operably linked to suitable regulatory sequences wherein expression of the chimeric gene results in an altered corn oleic acid phenotype.

In a fourth embodiment, this invention also concerns chimeric genes comprising an isolated nucleic acid fragment encoding a corn delta-9 stearoyl-ACP desaturase corresponding substantially to a nucleotide sequence set forth in any of SEQ ID NOS:8 and 10 or any functionally equivalent subfragment thereof or the reverse complement of such fragment or subfragment and an isolated nucleic acid fragment encoding a corn delta-12 desaturase or any functionally equivalent subfragment or the reverse complement of such fragment or subfragment which are operably linked and wherein expression of such combinations results in an altered corn oil phenotype.

Any of these chimeric genes may further comprise an isolated nucleic acid fragment comprising a corn oleosin promoter wherein said promoter can be full length or partial and further wherein said promoter comprises a nucleotide sequence corresponding substantially to the nucleotide sequence in any of SEQ ID NOS: 19 or 38-49 or said promoter comprises a fragment or subfragment that is substantially similar and functionally equivalent to any of the nucleotide sequences set forth in SEQ ID NOS:19 or 38-49 or a shrunken 1 intronl/exonl, or both.

Also included in this invention are corn plants and plant parts thereof containing the various chimeric genes, seeds of such plants, oil obtained from the grain of such plants, animal feed derived from the processing of such grain, the use of the foregoing oil in food, animal feed, cooking oil or industrial applications, products made from the hydrogenation, fractionation, interesterification or hydrolysis of such oil and methods for improving the carcass quality of an animal.

### BRIEF DESCRIPTION OF THE SEOUENCE LISTINGS AND FIGURES

The invention can be more fully understood from the following detailed description and the Figure and Sequence Descriptions which form a part of this application.

The sequence descriptions summarize the Sequences Listing attached hereto. The Sequence Listing contains one letter codes for nucleotide sequence characters and the three letter codes for amino acids as defined in the IUPAC-IUB standards described in Nucleic Acids Research 13:3021-3030 (1985) and in the Biochemical Journal 219 (No. 2):345-373 (1984), and the symbols and format used for all nucleotide and amino acid sequence data further comply with the rules governing nucleotide and/or amino acid sequence disclosures in patent applications as set forth in 37 C.F.R. §1.821-1.825 and WIPO Standard St.25.

SEQ ID NO:1 is a 1790 nucleotide sequence obtained from a corn cDNA which encodes a delta-12 desaturase enzyme (fad2-1). This sequence is also set forth in WO 94/11516.

SEQ ID NO:2 is a 1733 nucleotide sequence obtained from a corn cDNA which encodes a second delta-12 desaturase enzyme (fad2-2).

SEQ ID NO:3 is the translation product of the nucleotide sequence set forth in SEQ ID NO:2. The translation product is a polypeptide of 392 amino acids (translation frame: nucleotides 176-1351).

SEQ ID NO:4 is a 12,313 nucleotide sequence obtained from corn genomic DNA which comprises the region upstream of the fad2-2 coding region.

SEQ ID NO:5 is 2,907 nucleotide sequence obtained from corn genomic DNA which includes the fad2-1 intron.

SEQ ID NO:6 is a 18 base oligonucleotide primer used to amplify corn delta-9 desaturase via PCR.

SEQ ID NO:7 is a 17 base oligonucleotide primer used to amplify corn delta-9 desaturase via PCR.

SEQ ID NO:8 is the 1714 nucleotide sequence of a corn delta-9 desaturase cDNA as contained in plasmid pCD520.

SEQ ID NO:9 is the translation product of the nucleotide sequence set forth in SEQ ID NO:8. The translation product is a polypeptide of 392 amino acids (translation frame: nucleotides 134-1312).

SEQ ID NO: 10 is a 1709 nucleotide sequence of a second corn delta-9 desaturase cDNA as contained in plasmid pBN408.

SEQ ID NO: 11 is the translation product of the nucleotide sequence set forth in SEQ ID NO:10. The translation product is a polypeptide of 392 amino acids (translation frame: nucleotides 102-1280).

SEQ ID NO: 12 is a 18 base oligonucleotide primer used to amplify a portion of corn fad2-1 via PCR.

SEQ ID NO:13 is a 17 base oligonucleotide primer used to amplify a portion of corn fad2-1 via PCR.

SEQ ID NOS:14 and 15 are 21 base oligonucleotide primers used to amplify a portion of the oleosin 16 kDa gene via PCR.

SEQ ID NOS:16 and 17 are 22 and 20, respectively, base oligonucleotide primers used to amplify a portion of the oleosin 18 kDa gene via PCR.

SEQ ID NO:18 is a 46 base oligonucleotide used as a hybridization probe to identify oleosin genes.

SEQ ID NO:19 is a 1714 nucleotide sequence of a corn oleosin 16 kDa promoter.

SEQ ID NO:20 is a 32 base oligonucleotide primer used to amplify deletion derivatives of the oleosin 16 kDa promoter via PCR.

SEQ ID NO:21 is a 33 base oligonucleotide primer used to amplify deletion derivatives of the oleosin 16 kDa promoter via PCR.

SEQ ID NO:22 is a 33 base oligonucleotide primer used to amplify deletion derivatives of the oleosin 16 kDa promoter via PCR.

SEQ ID NO:23 is a 32 base oligonucleotide primer used to amplify deletion derivatives of the oleosin 16 kDa promoter via PCR.

SEQ ID NO:24 is a 37 base oligonucleotide primer used to amplify deletion derivatives of the oleosin 16 kDa promoter via PCR.

SEQ ID NO:25 is a 32 base oligonucleotide primer used to amplify deletion derivatives of the oleosin 16 kDa promoter via PCR.

SEQID NO:26 is a 32 base oligonucleotide primer used to amplify deletion derivatives of the oleosin 16 kDa promoter via PCR.

SEQ NO:27 is a 33 base oligonucleotide primer used to amplify deletion derivatives of the oleosin 16 kDa promoter via PCR.

SEQ ID NO:28 is a 24 base oligonucleotide primer used to amplify deletion derivatives of the oleosin 16 kDa promoter via PCR.

SEQID NO:29 is a 19 base oligonucleotide primer used to amplify deletion derivatives of the oleosin 16 kDa promoter via PCR.

SEQ ID NO:30 is a 25 base oligonucleotide primer used to amplify the shrunken 1 intronl/exonl via PCR.

SEQ ID NO:31 is a 25 base oligonucleotide primer used to amplify the shrunken 1 intron1/exon1 via PCR.

SEQ ID NOS:32 and 33 are 30 base oligonucleotides used as hybridization probes to identify clones containing the globulin-1 gene.

SEQ ID NOS:34 and 35 are 30 base oligonucleotide primers used to amplify the globulin-I promoter.

SEQ ID NOS:36 and 37 are 36 and 39, respectively, base oligonucleotide primers used to amplify the globulin-I promoter.

SEQ ID NO:38 is a 1.1 kb deletion derivative of the oleosin 16 kDa promoter.

SEQ ID NO:39 is a 0.9 kb deletion derivative of the oleosin 16 kDa promoter.

SEQ ID NO:40 is a 0.55 kb deletion derivative of the oleosin 16 kDa promoter.

SEQ ID NO:41 is a 0.95 kb deletion derivative of the oleosin 16 kDa promoter.

SEQ ID NO:42 is a 1.4 kb deletion derivative of the oleosin 16 kDa promoter.

SEQ ID NO:43 is a 1.0 kb deletion derivative of the oleosin 16 kDa promoter.

SEQ ID NO:44 is a 0.75 kb deletion derivative of the oleosin 16 kDa promoter.

SEQ ID NO:45 is a 0.4 kb deletion derivative of the oleosin 16 kDa promoter.

SEQ ID NO:46 is a 1.3 kb deletion derivative of the oleosin 16 kDa promoter.

SEQ ID NO:47 is a 0.8 kb deletion derivative of the oleosin 16 kDa promoter.

SEQ ID NO:48 is a 0.6 kb deletion derivative of the oleosin 16 kDa promoter.

SEQ ID NO:49 is a 0.3 kb deletion derivative of the oleosin 16 kDa promoter.

SEQ ID NOS:50 and 51 are 29 base oligonucleotide primers used to amplify the fad2-1 coding region via PCR.

SEQ ID NOS:52 and 53 are 31 and 30, respectively, base oligonucleotide primers used to amplify the delta-9 desaturase coding region via PCR.

SEQID NO:54 and 55 are 20 and 25, respectively, base oligonucleotide primers used to amplify portions of the fad2 genes via PCR.

SEQ ID NO:56 and 57 are 20 base oligonucleotide primers used to amplify the fad2-1 intron via PCR.

SEQID NO:58 is the complete nucleotide seqquece of plasmid pBN257. It contains an out-of-frame translation start for fad2-1 beginning at position 1978.

SEQ ID NO:59 is a truncated form of the fad2-1 gene from pBN257. The coding frame from pBN257 is represented by nucleotides 1991-3136 of SEQ ID NO:58.

FIG. 1 depicts Northern blot analyses of the developmental regulation of genes that are highly expressed in embryo and aleurone. Individual blots used the following as probes: FIG. 1A, fad2-1; FIG. 1B, delta-9 desaturase; FIGS. 1C and 1D, globulin-1, and FIGS. 1E and 1F, oleosin 16 kDa.

FIG. 2A depicts a restriction map of plasmid pML63.

FIG. 2B depicts a restriction map of plasmid pSH12.

FIG. 2C depicts a restriction map of plasmid pSM100.

FIG. 3A depicts a restriction map of plasmid pBN256.

FIG. 3B depicts a restriction map of plasmid pBN257.

FIG. 3C depicts a restriction map of plasmid pBN264.

FIG. 3D depicts a restriction map of plasmid pBN262.

FIG. 3E depicts a restriction map of plasmid pBN414.

FIG. 3F depicts a restriction map of plasmid pBN412.

FIG. 4A depicts the lipid profiles of individual kernels obtained from corn line FA013-2-4.

FIG. 4B is a histogram depicting the segregation analysis of the lipid profiles of individual kernels obtained from corn line FA013-2-4.

FIG. 5 depicts the lipid profiles of individual R2 kernels obtained from corn line FA013-3-2-15.

FIG. 6 depicts the lipid profiles of individual R1 kernels obtained from corn line FA014-5-1.

FIG. 7A depicts a restriction map of plasmid pBN427.

FIG. 7B depicts a restriction map of plasmid pBN428.

FIG. 7C depicts a restriction map of plasmid pBN431.

### DETAILED DESCRIPTION OF THE INVENTION

In the context of this disclosure, a number of terms shall be utilized.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric genes to produce the desired phenotype in a transformed plant. Chimeric genes can be designed for use in co-suppression or antisense by linking a nucleic acid fragment or subfragment thereof, whether or not it encodes an active enzyme, in the appropropriate orientation relative to a plant promoter sequence.

The terms “substantially similar” and “corresponding substantially” as used herein refer to nucleic acid fragments wherein changes in one or more nucleotide bases does not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

Moreover, the skilled artisan recognizes that substantially similar nucleic acid sequences encompassed by this invention are also defined by their ability to hybridize, under moderately stringent conditions (for example, 0.5× SSC, 0.1% SDS, 60° C.) with the sequences exemplified herein, or to any portion of the nucleotide sequences reported herein and which are functionally equivalent to the promoter of the invention. Preferred substantially similar nucleic acid sequences encompassed by this invention are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. Sequence alignments and percent similarity calculations may be determined using the Megalign program of the LASARGENE bioinformatics computing suite (DNASTAR Inc., Madison, Wis.). Multiple alignment of the sequences are performed using the Clustal method of alignment (Higgins and Sharp (1989) CABIOS. 5:151-153) with the default parameters (GAP PENALTY=10, GAP LENGTH PENALTY=10). Default parameters for pairwise alignments and calculation of percent identiy of protein sequences using the Clustal method are KTUPLE=1, GAP PENALTY=3, WINDOW=5 and DIAGONALS SAVED=5. For nucleic acids these parameters are GAP PENALTY=10, GAP LENGTH PENALTY=10, KTUPLE=2, GAP PENALTY=5, WINDOW=4 and DIAGONALS SAVED=4. A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F., et al., (1993) J. Mol. Biol. 215:403-410) and Gapped Blast (Altschul, S. F. et al., (1997) Nucleic Acids Res. 25:3389-3402); see also www.ncbi.nlm.nih.gov/BLAST/).

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg (1989, Biochemistry of Plants 15:1-82). It is further recognized that since in most cases the exact boundaries of regulatory sequences have not been completely defined, DNA fragments of some variation may have identical promoter activity.

An “intron” is an intervening sequence in a gene that does not encode a portion of the protein sequence. Thus, such sequences are transcribed into RNA but are then excised and are not translated. The term is also used for the excised RNA sequences. An “exon” is a portion of the sequence of a gene that is transcribed and is found in the mature messenger RNA derived from the gene, but is not necessarily a part of the sequence that encodes the final gene product.

The term “shrunken 1 intron/exon” refers to a region of the shrunken 1 gene from corn. The particular intron/exon used in the present invention is derived from a non-coding region (“exon 1/intron 1”) of the shrunken 1 gene and is identical to the sequence in GenBank accession #X02382 from nucleotides 1138 through 2220. As used herein, the terms shrunken 1 and its abbreviation, Sh1, are used interchangably.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The expression “3′ non-coding sequences” refers to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of an mRNA precursor. The use of different 3′ non-coding sequences is exemplified by Ingelbrecht et al. (1989, Plant Cell 1:671-680).

“RNA transcript” refers to a product resulting from RNA polymerase-catalyzed transcription of a DNA sequence. When an RNA transcript is a perfect complementary copy of a DNA sequence, it is referred to as a primary transcript or it may be a RNA sequence derived from posttranscriptional processing of a primary transcript and is referred to as a mature RNA. “Messenger RNA” (“mRNA”) refers to RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded by using the klenow fragment of DNA polymerase I. “Sense” RNA refers to RNA transcript that includes mRNA and so can be translated into protein within a cell or in vitro. “Antisense RNA” refers to a RNA transcript that is complementary to all or part of a target primary transcript or mRNA and that blocks expression or transcripts accumulation of a target gene (U.S. Pat. No. 5,107,065). The complementarily of an antisense RNA may be with any part of the specific gene transcript, i.e. at the 5′ non-coding sequence, 3′ non-coding sequence, introns, or the coding sequence. “Functional RNA” refers to antisense RNA, ribozyme RNA, or other RNA that may not be translated but yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence, i.e., that the coding sequence is under the transcriptional control of the promoter. Coding sequences can be operably linked to regulatory sequences in sense or antisense orientation.

The term “expression”, as used herein, refers to the production of a functional end-product. Expression or overexpression of a gene involves transcription of the gene and translation of the mRNA into a precursor or mature protein. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression or transcript accumulation of identical or substantially similar foreign or endogenous genes (U.S. Pat. No. 5,231,020). The mechanism of co-suppression may be at the DNA level (such as DNA methylation), at the transcriptional level, or at post-transcriptional level.

“Altered expression” refers to the production of gene product(s) in transgenic organisms in amounts or proportions that differ significantly from that activity in comparable tissue (organ and of developmental type) from wild-type organisms.

“Mature” protein refers to a post-translationally processed polypeptide, i.e., one from which any pre- or propeptides present in the primary translation product have been removed. “Precursor” protein refers to the primary product of translation of mRNA, i.e., with pre- and propeptides still present. Pre- and propeptides may be but are not limited to intracellular localization signals.

A “chloroplast transit peptide” is an amino acid sequence which is translated in conjunction with a protein and directs the protein to chloroplasts or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels, J. J., (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632).

“Delta-9 desaturase” (alternatively, “stearoyl-ACP desaturase”) catalyzes the introduction of a double bond between carbon atoms 9 and 10 of stearoyl-ACP to form oleoyl-ACP. It can also convert stearoyl-CoA into oleoyl-CoA, albeit with reduced efficiency.

“Delta-12 desaturase” refers to a fatty acid desaturase that catalyzes the formation of a double bond between carbon positions 6 and 7 (numbered from the methyl end), (i.e., those that correspond to carbon positions 12 and 13 (numbered from the carbonyl carbon) of an 18 carbon-long fatty acyl chain.

As used herein, the expressions “nucleic acid fragment encoding a corn delta-9 desaturase” and “nucleic acid fragment encoding a corn delta-12 desaturase” refer to nucleic acid fragments that are derived from a desaturase cDNA or genomic sequence, but which may or may not produce active enzymes. For example, such a fragment could be a mutant sequence that does not give rise to a translated product, or coding frame has been shifted that may give rise to a different polypeptide, but which is functional for the alteration of desaturase enzyme level. In other words, such a fragment could be used in the construction of a co-suppression or antisense chimeric gene to alter desaturase enzyme level and, thus, alter the lipid profile of a plant transformed with such a chimeric gene.

“Transformation” refers to the transfer of a nucleic acid fragment into the genome of a host organism, resulting in genetically stable inheritance. Host organisms containing the transformed nucleic acid fragments are referred to as “transgenic” organisms. The preferred method of corn cell transformation is use of particle-accelerated or “gene gun” transformation technology (Klein K. et al. (1987) Nature (London) 327:70-73; U.S. Pat. No. 4,945,050), or Agrobacterium-mediated method using an appropriate Ti plasmid containing the transgene (Ishida Y. et al. 1996, Nature Biotech. 14:745-750). The expression “transgenic event” refers to an independent transgenic line that is derived from a single callus clone containing a transgene.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the the target segment are annealed at low temperature and then extended at an intermediate temperature. One set of these three consecutive steps comprises a cycle. An “expression construct” is a plasmid vector or a subfragment thereof comprising the instant chimeric gene. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

An “RO” plant is equivalent to a “primary transformant,” which is the plant regenerated directly from the tissue culture processes after transformation by the biolistic or Agrobacterium-mediated method. Seeds harvested from RO plants, were named R1 or RO:1 seeds. Progenies derived from R1 seeds are R1 plants, and seeds harvested from R1 plants are R2 or R1:2 seeds. Future generations are named according to this convention.

The “kernel” is the corn caryopsis, consisting of a mature embryo and endosperm which are products of double fertilization. The term “corn” or “maize” represents any variety, cultivar, or population of Zea mays L.

“Grain” comprises mature corn kernels produced by commercial growers for on farm use or for sale to customers in both cases for purposes other than growing or reproducing the species. The “seed” is the mature corn kernel produced for the purpose of propagating the species and for sale to commercial growers. As used herein the terms seeds, kernels, and grains can be used interchangeably. The “embryo” or also termed “germ” is a young sporophytic plant, before the start of a period of rapid growth (seed germination). The embryo (germ) of corn contains the vast majority of the oil found in the kernel. The structure of embryo in cereal grain includes the embryonic axis and the scutellum. The “scutellum” is the single cotyledon of a cereal grain embryo, specialized for absorption of the endosperm. The “aleurone” is a proteinaceous material, usually in the form of small granules, occurring in the outermost cell layer of the endosperm of corn and other grains.

A “dominant” trait requires one allele to be dominant with respect to an alternative allele if an individual cell or organism homozygous for the dominant allele is phenotypically indistinguishable from the heterozygote. The other, alternative allele is said to be recessive. “Recessive” describes a gene whose phenotypic expression is masked in the heterozygote by a dominant allele. “Semi-dominant” describes an intermediate phenotype in a heterozygote. The term “homozygous” describes a genetic condition existing when identical alleles reside at corresponding loci on homologous chromosomes. The term “heterozygous” describes a genetic condition existing when different alleles reside at corresponding loci on homologous chromosomes.

As used herein in describing “oleic acid content”, the term “high oleate” refers to a grain or seed having an oleic acid content of not less than about 60% of the total oil content of the seed, by weight when measured at 0% moisture. “Stearic acid content”, the term “high stearate” refers to a grain or seed having an stearic acid content of not less than about 20% of the total oil content of the seed, by weight when measured at 0% moisture. “Saturated fatty acid” is a fatty acid that contains a saturated alkyl chain. The term “high saturate” refers to a grain or seed having an total saturated fatty acid content of not less than about 30% of the total oil content of the seed, by weight when measured at 0% moisture. The major components of the saturated fatty acid fraction of a grain or seed include but not limited to palmitic (16:0), stearic (18:0), and arachidic (20:0) acids.

A “carcass quality improving amount” is that amount needed to improve the carcass quality of an animal. The present invention concerns the alteration of lipid profiles in corn.

In one aspect this invention concerns an isolated nucleic acid fragment comprising a corn oleosin promoter wherein said promoter can be full length or partial and further wherein said promoter comprises a nucleotide sequence corresponding substantially to the nucleotide sequence in any of SEQ ID NOS:19 or 38-49 or said promoter comprises a fragment or subfragment that is substantially similar and functionally equivalent to any of the nucleotide sequences set forth in SEQ ID NOS:19 or 38-49. In addition, the fragment or subfragment discussed above may hybridize to the nucleotide sequence set forth in SEQ ID NOS:19 or 38-49 under moderately stringent conditions. This novel corn oleosin promoter is capable of driving gene expression in an embryo and aleurone-specific manner at a high expression level. Strong promoter activity in developing corn embryos is best achieved by using the nucleic acid fragment corresponding substantially to the nucleotide sequence set forth in SEQ ID NO:39 and an intron element in the expression construct as discussed in the examples below. It has been found that the activity of oleosin promoter is much higher, and expressed much earlier in the developing corn kernels, than a corn embryo/aleurone-specific promoter obtained from the globulin-1 gene. The preferred oleosin promoter has the nucleotide sequence set forth in SEQ ID NO:39. However, as those skilled in the art will appreciate, any functional promoter which has embryo/aleurone specificity is useful in the present invention. Other suitable promoters are well known to those skilled in the art, examples of which are discussed in WO 94/11516, the disclosure of which is hereby incorporated by reference. Furthermore, one skilled in the art will be able to use the methods and analyses that are described in the Examples below to identify other promoters with the desired embryo/aleurone specificity of expression. For example, using the instant optimized oleosin promoter as a contol, it is possible to identify other sequences that function in a similar manner, using the histological and molecular biological characterizations of embryo/aleurone promoter function, such as levels of expression of a GUS reporter function, timing of gene expression that is comtemporaneous with seed oil formation, and the appropriate tissue specificity.

In a second embodiment, this invention concerns an isolated nucleic acid fragment encoding a corn delta-9 stearoyl-ACP desaturase corresponding substantially to a nucleotide sequence set forth in any of SEQ ID NOS:8 or 10 or any functionally equivalent subfragment thereof. Chimeric genes comprising this nucleic acid fragment or subfragment thereof or the reverse complement of such fragment or subfragment operably linked to suitable regulatory suitable regulatory sequences can be constructed wherein expression of the chimeric gene results in an altered corn stearic acid phenotype.

Transgenic plants can be made in which a corn delta-9 desaturase enzyme is present at higher or lower levels than normal or in cell types or developmental stages in which it is not normally found. This would have the effect of altering the level of delta-9 desaturases in those cells. It may be desirable to reduce or eliminate expression or transcript accumulation of a gene encoding delta-9 desaturases in plants for some applications. In order to accomplish this, a chimeric gene designed for co-suppression of the endogenous delta-9 desaturases can be constructed by linking a nucleic acid fragment or subfragment thereof encoding corn delta-9 desaturases to plant promoter sequences. Alternatively, a chimeric gene designed to express antisense RNA for all or part of the instant nucleic acid fragment can be constructed by linking the nucleic acid fragment or subfragment in reverse orientation to plant promoter sequences, i.e., by linking the reverse complement of the fragment or subfragment. Either the co-suppression or antisense chimeric genes could be introduced into plants via transformation wherein expression or transcript accumulation of the corresponding endogenous genes are reduced or eliminated (Stam, et al. (1997) Annals of Botany 79:3-12).

Expression of a trait gene in corn kernels may be accomplished by constructing a chimeric gene in which the coding region of the trait gene and other regulatory element (for example, intron) is operably linked to the oleosin 16 kDa promoter. The chimeric gene may comprise the shrunken 1 intronl/exonl in the 5′-untranslated sequence to either enhance the gene expression or stabilize the transcripts of the transgene. The Shl exon I sequence will remain as part of the leader sequences in mRNA after the splicing occurs. All or a portion of the coding sequence of the trait gene is located 3′ to the Shl exonl/intronl sequence, and may be in a sense or antisense orientation. Such a chimeric gene may also comprise one or more introns in order to facilitate gene expression. The position of the intron element(s) can be in the translation leader sequence as described above, or in the coding region of the trait gene. Intron elements from other genes, such as actin-1, ubiquitin-1, Adh-1, fad2-1, and fad2-2 may also be used in replacing the Shl element to have the same effect. Accordingly, any intron element from other genes may be used to practice the instant invention. 3′ non-coding sequences containing transcription termination signals may also be provided in the chimeric gene.

All or a portion of any of the nucleic acid fragments of the instant invention may also be used as a probe for genetically and physically mapping the genes that it is a part of, and as a marker for traits linked to these genes. Such information may be useful in plant breeding in order to develop lines with desired phenotypes. For example, such fragment may be used as a restriction fragment length polymorphism (RFLP) marker. Southern blots (Sambrook) of restriction-digested plant genomic DNA may be probed with the nucleic acid fragment of the instant invention. The resulting banding patterns may then be subjected to genetic analyses using computer programs such as MapMaker (Lander et al., (1987) Genomics 1:174-181) in order to construct a genetic map. In addition, the nucleic acid fragment of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein, D. et al., (1980) Am. J. Hum. Genet. 32:314-331).

The production and use of plant gene-derived probes for use in genetic mapping is described in R. Bernatzky, R. and Tanksley, S. D. (1986) Plant Mol. Biol. Reporter 4(1):37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequence may also be used for physical mapping (i.e., placement of sequences on physical maps; see Hoheisel, J. D., et al., In: Nonmammalian Genomic Analysis: A Practical Guide, Academic press 1996, pp. 319-346, and references cited therein).

In a third embodiment, this invention concerns an isolated nucleic acid fragment encoding a corn delta-12 desaturase corresponding su.bstantially to the nucleotide sequence set forth in SEQ ID NO:2 or any functionally equivalent subfragment thereof. The gene for microsomal delta-12 fatty acid desaturases described in WO 94/11516, published on May 26, 1994, can be used to practice the instant invention. Chimeric genes comprising such a nucleic acid fragment or subiragment thereof or the reverse complement of such fragment or subfragment operably linked to suitable regulatory sequences can be constructed wherein expression of the chimeric gene results in an altered corn oleic acid phenotype. As was discussed above with respect to an isolated nucleic acid fragment encoding a delta-9 desaturase, it may be desirable to reduce or eliminate expression or transcript accumulation of a gene encoding delta-12 desaturases in plants for some applications. To accomplish this, a chimeric gene designed for co-suppression of the endogenous delta-12 desaturases can be constructed by linking a nucleic acid fragment or subfragment thereof to plant promoter sequences. Alternatively, a chimeric gene designed to express antisense RNA for all or part of this nucleic acid fragment can be constructed by linking the nucleic acid fragment or subfragment in reverse orientation to plant promoter sequences, i.e., by linking the reverse complement of the fragment or subfragment to plant promoter sequences. Either the co-suppression or antisense chimeric genes can be introduced into plants via transformation wherein expression of the corresponding endogenous genes are reduced or eliminated.

The aforementioned chimeric genes can further comprise (1) an isolated nucleic acid fragment comprising a corn oleosin promoter wherein said promoter can be full length or partial and further wherein said promoter comprises a nucleotide sequence corresponding substantially to the nucleotide sequence in any of SEQ ID NOS:19 or 38-49 or said promoter comprises a fragment or subfragment that is substantially similar and functionally equivalent to any of the nucleotide sequences set forth in SEQ ID NOS:19 or 38-49 and/or (2) a shrunken 1 intron/exon.

In a further aspect, chimeric genes can be constructed to encompass a variety of combinations, including but not limited to the following:

a) A chimeric gene comprising an isolated nucleic acid fragment encoding a corn delta-9 stearoyl-ACP desaturase corresponding substantially to a nucleotide sequence set forth in any of SEQ ID NOS:8 or 10 or any functionally equivalent subfragment thereof or the reverse complement of this fragment or subfragment and a nucleic acid fragment encoding a corn delta-12 desaturase or any functionally equivalent subfragment thereof or the reverse complement of this fragment or subfragment wherein the fragments or subfragment are operably linked and further wherein expression of this chimeric gene results in an altered corn oil phenotype.

The nucleic acid fragment encoding a corn delta-12 desaturase enzyme used in the contraction of such a chimeric gene can be the fragment identified in WO 94/11516 or this fragment can correspond substantially to the nucleotide sequence set forth in SEQ ID NO:2 or any functionally equivalent subfragment thereof.

b) The chimeric gene described in (a) above can still further comprise an isolated nucleic acid fragment comprising a corn oleosin promoter wherein said promoter can be full length or partial and further wherein said promoter comprises a nucleotide sequence corresponding substantially to the nucleotide sequence in any of SEQ ID NOS:19 or 38-49 or said promoter comprises a fragment or subfragment that is substantially similar and functionally equivalent to any of the nucleotide sequences set forth in SEQ ID NOS: 19 or 38-49.

c) The chimeric gene described in (a) or (b) above can each further comprise a shrunken 1 intron/exon.

d) A chimeric gene comprising (1) an isolated nucleic acid fragment comprising a corn oleosin promoter wherein said promoter can be full length or partial and further wherein said promoter comprises a nucleotide sequence corresponding substantially to the nucleotide sequence in any of SEQ ID NOS: 19 or 38-49 or said promoter comprises a fragment or subfragment that is substantially similar and functionally equivalent to any of the nucleotide sequences set forth in SEQ ID NOS:19 or 38-49, (2) an isolated nucleic acid fragment encoding a corn delta-9 stearoyl-ACP desaturase corresponding substantially to a nucleotide sequence set forth in any of SEQ ID NOS:8 or 10 or a functionally equivalent subfragment thereof or the reverse complement of the fragment or subfragment, (3) a nucleic acid fragment encoding a corn delta-12 desaturase or any functionally equivalent subfragment thereof, thereof or the reverse complement of the fragment or subfragment, and (4) a shrunken 1 intron/exon wherein the fragments are operably linked and further wherein expression of this chimeric gene results in an altered corn oil phenotype. In another embodiment, the nucleic acid fragment encoding the delta-12 desaturase corresponds substantially to the nucleotide sequence set forth in SEQ ID NO:2.

e) A chimeric gene comprising (1) an isolated nucleic acid fragment comprising a corn oleosin promoter wherein said promoter can be full length or partial and further wherein said promoter comprises a nucleotide sequence corresponding substantially to the nucleotide sequence in any of SEQ ID NOS: 19 or 38-49 or said promoter comprises a fragment or subfragment that is substantially similar and functionally equivalent to any of the nucleotide sequences set forth in SEQ ID NOS: 19 or 38-49, (2) a nucleic acid fragment encoding a corn delta-12 desaturase corresponding substantially to the nucleotide sequence set forth in SEQ ID NO: 1 or any functionally equivalent subfragment thereof, or the reverse complement of this fragment or subfragment, or an isolated nucleic acid fragment corresponding substantially to the nucleotide sequence set forth in SEQ ID NO:58 or 59 or any functionally equivalent subfragment thereof, or the reverse complement of this fragment or subfragment and a shrunken 1 intron/exon wherein the fragments are operably linked and further wherein expression of this chimeric gene results in an altered corn oil phenotype. In another embodiment, the nucleic acid fragment encoding the delta-12 desaturase corresponds substantially to the nucleotide sequence set forth in SEQ ID NO:2.

This invention also concerns corn plants and plant parts thereof comprising in their genome these various chimeric genes. Corn grains obtained from such plants will have altered corn oil phenotypes. For example, a corn grain obtained from a corn plant comprising in its genome a chimeric gene comprising an isolated nucleic acid fragment encoding a corn delta-9 stearoyl-ACP desaturase corresponding substantially to a nucleotide sequence set forth in any of SEQ ID NOS:8 or 10 or any functionally equivalent subfragment thereof or the reverse complement of this fragment or subfragment operably linked to suitable regulatory sequences will have a stearic acid content of not less than about 20% of the total oil content or a total saturate content of not less than about 35% of the total oil content. The preferred regulatory sequence is the oleosin promoter. This same phenotype will be obtained if this chimeric gene further comprises an isolated nucleic acid fragment encoding a corn delta-9 stearoyl-ACP desaturase corresponding substantially to a nucleotide sequence set forth in any of SEQ ID NOS:8 or 10 or any functionally equivalent subfragment thereof or the reverse complement of this fragment or subfragment and/or a shrunken 1 intron/exon.

A corn grain comprising in its genome a chimeric gene comprising an isolated nucleic acid fragment comprising a corn delta-12 desaturase corresponding substantially to the nucleotide sequence set forth in SEQ ID NO: 1, a functionally equivalent subfragment thereof or the reverse complement of said fragment or subfragment, or an isolated nucleic acid fragment corresponding substantially to the nucleotide sequence set forth in SEQ ID NO:58 or 59 or a functionally equivalent subfragment thereof or the reverse complement of such fragment or subfragment, an isolated nucleic acid fragment comprising a corn oleosin promoter wherein said promoter can be full length or partial and further wherein said promoter comprises a nucleotide sequence corresponding substantially to the nucleotide sequence in any of SEQ ID NOS:19 or 38-49 or said promoter comprises a fragment or subfragment that is substantially similar and functionally equivalent to any of the nucleotide sequences set forth in SEQ ID NOS:19 or 38-49, and shrunken 1 intron/exon wherein said fragments are operably linked and further wherein expression of the chimeric gene results in an altered corn oleic acid phenotype, wherein said corn grain has an oil content in the range from about 6% to about 10% on a dry matter basis and further wherein said oil is comprised of not less than about 60% oleic acid based on the total oil content of the seed.

Such a corn grain can be obtained by the Top Cross® grain production method cited in the Examples below. In this method one of the parents comprises the chimeric gene discussed above and the other parent comprises a high oil phenotype in the range from about 12% to 20% oil by weight or on a dry matter basis. Alternatively, one of the parents may comprise both a transgene of the invention, e.g., a chimeric gene of this invention, and a high oil phenotype, and the other parent is an elite hybrid line.

A corn grain obtained from a corn plant comprising in its genome a chimeric gene comprising an isolated nucleic acid fragment encoding a corn delta-12 desaturase corresponding substantially to the nucleotide sequence set forth in SEQ ID NO:2 or any functionally equivalent subfragment thereof or the reverse complement of the fragment or subfragment operably linked to suitable regulatory sequences will have an oleic acid content of not less than about 70% of the total oil content. The preferred regulatory sequence is the oleosin promoter. This same phenotype will be obtain if this chimeric gene further comprises an isolated nucleic acid fragment encoding a corn delta-9 stearoyl-ACP desaturase corresponding substantially to a nucleotide sequence set forth in any of SEQ ID NOS:8 or 10 or any functionally equivalent subfragment thereof thereof or the reverse complement of the fragment or subfragment and/or a shrunken 1 intron/exon.

With respect to the chimeric genes discussed above in (a) through (e), comprising the various gene combinations, corn grains obtained from plants comprising such chimeric genes will have a total saturate content of not less than about 30% of the total oil content and an oleic acid content of not less than about 30% of the total oil content.

This invention also concerns obtained from corn plants containing any of the above-discussed chimeric genes, oil obtained from such seeds, animal feed derived from the processing of such seeds, the use of such oil in food, animal feed, cooking or industrial applications and products made from the hydrogenation, fractionation, interesterification or hydrolysis of such oil, by-products made during the production of this oil, and methods for improving the carcass quality of animals.

The present invention also concerns a method for improving the carcass quality of an animal which comprises feeding the animal a carcass quality improving amount of animal feed derived from the processing of corn seeds/grain obtained from any of the corn plants of the present invention.

Vegetable oils are often used in high temperature applications. Oil oxidation is accelerated in the presence of heat. It is important that an oil be able to withstand these conditions for applications such as frying, baking, roasting, etc. In some cases, antioxidants may be added to improve stability but not all antioxidants withstand high temperatures. In addition, in many cases a food manufacturer does not want to use oils with added antioxidants if a label with unadulterated ingredients is desired. Therefore, an oil which is stable to oxidation under high temperatures in the absence of any additives or other processing is highly desirable. Overheating of oils often leads to thermal polymerization of the oil and oxidation products resulting in-a gummy, varnish-like buildup on the equipment used for heating and excessive foaming of the oil. As a result of oxidation, a variety of degradation products are formed depending on the conditions under which the oil is exposed. High temperature stability can be evaluated by exposing the oils to high temperature and monitoring the formation of the undesirable degradation products. These include both volatile and nonvolatile products and may be hydrocarbons, alcohols, aldehydes, ketones, and acids. The nonvolatile components can be further classified into polar and polymerized compounds. The polar and polymerized compounds present in a degraded oil can be analyzed directly by reverse phase high performance liquid chromatography as described in Lin, S. S., 1991, Fats and oils oxidation. Introduction to Fats and Oils Technology (Wan, P. J. ed.), pages 211-232, Am. Oil Chem. Soc.

The oil of this invention can be used in a variety of applications. In general, oxidative stability is related to flavor stability. The oil of this invention can be used in the preparation of foods. Examples include, but are not limited to, uses as ingredients, as coatings, as salad oils, as spraying oils, as roasting oils, and as frying oils. Foods in which the oil may be used include, but are not limited to, crackers and snack foods, confectionery products, syrups and toppings, sauces and gravies, soups, batter and breading mixes, baking mixes and doughs. Foods which incorporate the oil of this invention may retain better flavor over longer periods of time due to the improved stability against oxidation imparted by this oil.

The oils of this invention can also be used as a blending source to make a blended oil product. By a blending source, it is meant that the oil of this invention can be mixed with other vegetable oils to improve the characteristics, such as fatty acid composition, flavor, and oxidative stability, of the other oils. The amount of oil of this invention which can be used will depend upon the desired properties sought to be achieved in the resulting final blended oil product. Examples of blended oil products include, but are not limited to, margarines, shortenings, frying oils, salad oils, etc.

In another aspect, this invention concerns the industrial use of the oil of this invention for industrial applications such as an industrial lubricant for a variety of end uses, as a hydraulic fluid, etc. The industrial use of vegetable oils as a base fluid for lubricants has been known for many years. However, interest has intensified due to environmental concerns over the use of petroleum oils in environmentally sensitive areas. Vegetable oils are readily biodegradable, have low toxicity and have good lubricant characteristics. However, high pour points and rapid oxidation at high temperatures limit their use. Since the oil of this invention is low in polyunsaturates, as discussed herein, and has high oxidative stability and high temperature stability, these characteristics also make the oil of this invention desirable for industrial applications such as an industrial fluid, i.e., as industrial lubricant or as a hydraulic fluid, etc. Additives which can be used to make industrial lubricants and hydraulic fluids are commercially available. Indeed, some additives have been specially formulated for use with high oleic vegetable oils. Additives generally contain antioxidants and materials which retard foaming, wear, rust, etc.

Oil is obtained from plants by a milling process. Corn oil is extracted from kernels through the use of a either a wet or dry milling process. Wet milling is a multi-step process involving steeping and grinding of the kernels and separation of the starch, protein, oil, and fiber fractions. A review of the maize wet milling process is given by S. R. Eckhoff in the Proceedings of the Fourth Corn Utilization Conference, Jun. 24-26, 1992, St. Louis, Mo., printed by the National Corn Growers Association, CIBA-GEIGY Seed Division and the United States Department of Agriculture. Dry milling is a process by which the germ and hull of the corn kernel are separated from the endosperm by the controlled addition of water to the grain and subsequent passage through a degerming mill and a series of rollers and sieves. The U.S. dry milling industry produces approximately 50 million pounds of crude corn oil per year, and the wet milling industry produces over one billion pounds of crude corn oil (Fitch, B. (1985) JAOCS 62(11):1524-1531). The resulting oil is called crude oil.

The crude oil may be degummed by hydrating phospholipids and other polar and neutral lipid complexes which facilitate their separation from the nonhydrating, triglyceride fraction. Oil may be further refined for the removal of impurities; primarily free fatty acids, pigments, and residual gums. Refining is accomplished by the addition of caustic which reacts with free fatty acid to form soap and hydrates phosphatides and proteins in the crude oil. Water is used to wash out traces of soap formed during refining. The soapstock byproduct may be used directly in animal feeds or acidulated to recover the free fatty acids. Color is removed through adsorption with a bleaching earth which removes most of the chlorophyll and carotenoid compounds. The refined oil can be hydrogenated resulting in fats with various melting properties and textures. Winterization (fractionation) may be used to remove stearine from the hydrogenated oil through crystallization under carefully controlled cooling conditions. Deodorization which is principally steam distillation under vacuum, is the last step and is designed to remove compounds which impart odor or flavor to the oil. Other valuable byproducts such as tocopherols and sterols may be removed during the deodorization process. Deodorized distillate containing these byproducts may be sold for production of natural vitamin E and other high value pharmaceutical products. Refined, bleached, (hydrogenated, fractionated) and deodorized oils and fats may be packaged and sold directly or further processed into more specialized products.

Hydrogenation is a chemical reaction in which hydrogen is added to the unsaturated fatty acid double bonds with the aid of a catalyst such as nickel. High oleic oil contains unsaturated oleic acid, linoleic acid, and minor amount of linolenic acids and each of these can be hydrogenated. Hydrogenation has two primary effects. First, the oxidative stability of the oil is increased as a result of the reduction of the unsaturated fatty acid content. Second, the physical properties of the oil are changed because the fatty acid modifications increase the melting point resulting in a semi-liquid or solid fat at room temperature.

There are many variables which affect the hydrogenation reaction which in turn alter the composition of the final product. Operating conditions including pressure, temperature, catalyst type and concentration, agitation and reactor design are among the more important parameters which can be controlled. Selective hydrogenation conditions can be used to hydrogenate the more unsaturated fatty acids in preference to the less unsaturated ones. Very light or brush hydrogenation is often employed to increase stability of liquid oils. Further hydrogenation converts a liquid oil to a physically solid fat. The degree of hydrogenation depends on the desired performance and melting characteristics designed for the particular end product. Liquid shortenings, used in the manufacture of baking products, solid fats and shortenings used for commercial frying and roasting operations, and base stocks for margarine manufacture are among the myriad of possible oil and fat products achieved through hydrogenation. A more detailed description of hydrogenation and hydrogenated products can be found in Patterson, H. B. W., 1994, Hydrogenation of Fats and Oils: Theory and Practice. The American Oil Chemists' Society.

Interesterification refers to the exchange of the fatty acyl moiety between an ester and an acid (acidolysis), an ester and an alcohol (alcoholysis) or an ester and ester (transesterification). Interesterification reactions are achieved using chemical or enzymatic processes. Random or directed transesterification processes rearrange the fatty acids on the triglyceride molecule without changing the fatty acid composition. The modified triglyceride structure may result in a fat with altered physical properties. Directed interesterfication reactions using lipases are becoming of increasing interest for high value specialty products like cocoa butter substitutes. Products being commercially produced using interesterification reactions include but are not limited to shortenings, margarines, cocoa butter substitutes and structured lipids containing medium chain fatty acids and polyunsaturated fatty acids. Interesterification is further discussed in Hui, Y. H.(1996, Bailey's Industrial Oil and Fat Products, Volume 4, John Wiley & Sons).

Fatty acids and fatty acid methyl esters are two of the more important oleochemicals derived from vegetables oils. Fatty acids are used for the production of many products such as soaps, medium chain triglycerides, polyol esters, alkanolamides, etc. Vegetable oils can be hydrolyzed or split into their corresponding fatty acids and glycerine. Fatty acids produced from various fat splitting processes may be used crude or more often are purified into fractions or individual fatty acids by distillation and fractionation. Purified fatty acids and fractions thereof are converted into a wide variety of oleochemicals, such as dimer and trimer acids, diacids, alcohols, amines, amides, and esters. Fatty acid methyl esters are increasingly replacing fatty acids as starting materials for many oleochemicals such as fatty alcohols, alkanolamides, a-sulfonated methyl esters, diesel oil components, etc. Glycerine is also obtained by the cleavage of triglycerides using splitting or hydrolysis of vegetable oils. Further references on the commercial use of fatty acids and oleochemicals may be found in Erickson, D. R., 1995, Practical Handbook of Soybean Processing and Utilization, The American Oil Chemists' Society, and United Soybean Board; Pryde, E. H., 1979, Fatty Acids, The American Oil Chemists' Society; and Hui, Y. H., 1996, Bailey's Industrial Oil and Fat Products, Volume 4, John Wiley & Sons.

As was discussed above, this invention includes a transgenic corn plant capable of producing grains having an oleic acid content of not less than about 60% of the total oil content. The high oleate trait is dominant. Therefore, the desired phenotype can be obtained if only one of the parental lines in the seeds or grains production scheme contains the trait gene. The timeline for commercial production of corn having elevated oleic levels can be greatly accelerated.

In addition, the transgenic high saturate trait is dominant. Therefore, the desired phenotype can be obtained if only one of the parental lines in the seeds or grains production scheme contains the trait gene. The timeline for commercial production of corn having elevated oleic levels can be greatly accelerated.The DNA sequence information set forth in the instant invention may be used to isolate cDNAs and genes encoding delta-9 and delta-12 desaturases from corn. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding the desaturases (either as cDNAs or genomic DNAs), could be isolated directly by using all or a portion of the instant nucleic acid sequences to create DNA hybridization probes which could be used to screen libraries employing methodology well known to those skilled in the art. Specific oligonucleotide probes based upon the instant nucleic acid sequences can be designed and synthesized by methods known in the art (Sambrook). Moreover, the entire sequences can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency. It is further well known to persons skilled in the art that minor alterations (substitutions, additions or deletions) may be created by the use of various in vitro mutagenesis protocols. In this manner, any of the nucleic acid fragments of the instant invention may be readily obtained.

### EXAMPLES

The present invention is further defined in the following EXAMPLES, in which all parts and percentages are by weight and degrees are Celsius, unless otherwise stated. From the above discussion and these EXAMPLES, one skilled in the art can ascertain the essential characteristics of this invention, and without departing from the spirit and scope thereof, can make various changes and modifications of the invention to adapt it to various usages and conditions.

### Example 1

### Corn fad2-2 cDNA and Genomic DNA Clones

A corn embryo cDNA library was screened using a radioisotopically-labeled DNA fragment obtained by PCR and containing the com gene for delta-12 desaturase (“fad2-1”, WO 94/11516, and set forth in SEQ ID NO:1). A second delta-12 desaturase cDNA clone was identified on the basis of its sequence. The second gene for delta-12 desaturase is designated fad2-2.

The full-length cDNA sequence is shown in SEQ ID NO:2. It encodes a polypeptide of 392 amino acids (translation frame: nucleotide 176-1351). The coding region of the corn fad2-2 shares significant sequence identity with fad2-1: they share 88% identify at the amino acid level, and 92% at the nucleotide level. They also possess 77% identity at the 5′-untranslated region, and 64% at the 3′ end.

A full-length or a portion of the coding region of either one of genes in either antisense or sense approach may be used to suppress both the fad2-1 and fad2-2 genes or gene products, due to the significant homology in the coding region between the fad2-1 and fad2-2 genes, and thus produce a high oleate phenotype in transgenic corn.

A genomic clone with a 13 kb insert containing the fad2-2 gene was identified using the corn fad2-1 cDNA insert as a probe in a screen of a corn genomic DNA library (Mol 7 line, in λFix II vector, Stratagene, La Jolla, Calif.). The sequence upstream of the coding region is shown in SEQ ID NO:4, which contains the upstream regulatory element, 5′-untranslated region, and a 6.7 kb intron (nucleotide position at 5651-12301) located inside the 5′-untranslated region. The intron splice site (/GT-AGI) is conserved. The 5′-leader sequence (nucleotide position 5492-5650, and 12302-12313) flanking the intron matches the sequence of the 5′-untranslated region of fad2-2 cDNA. The putative TATA box (TAAATA) is at position 5439-5444, which is 47 nucleotides upstream from the first nucleotide of the fad2-2 cDNA clone. The promoter element of this gene may be used to express a gene of interest in transgenic corn plants.

### Example 2

### Corn fad2-1 Intron

Based on the fad2-2 intron sequence (SEQ ID NO:4), primers (SEQ ID NOS:54 and 55) were designed for PCR amplification of a fad2-2 fragment from corn genomic DNA for use in mapping the fad2-2 locus.

| | |
|---|---|
| 5′-CTGCACTGAAAGTTTTGGCA-3′ | SEQ ID NO:54 |
| 5′-AGTACAGCGGCCAGGCGGCGTAGCG-3′ | SEQ ID NO:55 |

In addition to the expected 0.8 kb fragment that should result from amplification from the fad2-2 sequence, a second fragment, 1.1 kb in length, was also produced in the same PCR. The 1.1 kb fragment was purified, sequenced, and it was determined that this fragment contains a portion of the fad2-1 intron. A new set of primers (SEQ ID NOS:56 and 57) were designed according to the sequences of this 1.1 kb partial intron, and the 5′-untranslated region of fad2-1.

| | |
|---|---|
| 5′-AAGGGGAGAGAGAGGTGAGG-3′ | SEQ ID NO:56 |
| 5′-TGCATTGAAGGTGGTGGTAA-3′ | SEQ ID NO:57 |

Using the new primer set and corn genomic DNA as the template, a PCR product containing the other half of the fad2-1 intron was obtained. The fragment was purified and sequenced. A contig containing the complete fad2-1 intron was assembled using the sequence that overlaps with the 1.1 kb fragment. The contig is shown in SEQ ID NO:5.

Comparison of the structures of corn fad2-1 and fad2-2 genes revealed that the locations of the introns are conserved. Both of the introns are localized to the 5′-leader region of the precursor RNA. The fad2-1 intron is 11 bases upstream of the start codon (ATG), whereas the fad2-2 intron is 27 bases upstream of the start codon. The consensus sequences of intron splice sites (/GT - - - AG/) are conserved in both introns.

Comparison of the fad2-1 and fad2-2 introns using the BestFit program (Genetics Computer Group, Madison, Wis.; employing the algorithm of Smith and Waterman (1981) Advances in Applied Mathematics 2:482-489) demonstrated 81% sequence identity in the first 0.76 kb (nucleotide positions 3-765 in the fad2-1 intron [SEQ ID NO:5]) and nucleotides 5650-6790 of the fad2-2 intron [as shown in SEQ ID NO:4]), and 73% homology near the end of the intron (nucleotide positions 2619-2893 in the fad2-1 intron [SEQ ID NO:5]), and 12006-12320 in the fad2-2 intron [SEQ ID NO:4]). The internal intron sequences are not conserved.

Very few plant introns studied to date are longer than 2-3 kb (Simpson and Filipowicz (1996) Plant Mol. Biol. 32:1-41). Further investigation indicated that the unusually large size of the fad2-2 intron was due to insertion of an apparently intact copy (about 4.8 kb) of a retrotransposable element, Milt (SanMiguel et al. (1996) Science 274:765-768). This retroelement is inserted in an opposite orientation of the transcription direction of the fad2-2 gene. The fad2-1 intron does not contain this element.

### Example 3

### Cloning and Sequencing of Corn Delta-9 Desaturase cDNA

Degenerate primers were designed according to the conserved regions of delta-9 desaturase genes from various species, and used for PCR. These are set forth in SEQ ID NOS:6 and 7.

| | |
|---|---|
| 5′-GAYATGATHACNGARGAR-3′ | SEQ ID NO:6 |
| 5′-CCRTCRTACATNAGATG-3′ | SEQ ID NO:7 |

Two PCR fragments (520 and 500 bp, respectively) were generated when these oligomers were used as primers and DNA from a corn embryo cDNA library was used as a template. The fragments were purified and used as probes to screen a corn embryo cDNA library. Two independent clones (pCD520, and pCD500) were isolated.

These two clones were sequenced, and cross-hybridized between themselves and with the soybean delta-9 desaturase gene. It was confirmed that only the insert of pCD520 was homologous to the soybean delta-9 desaturase gene. The cDNA sequence was shown in SEQ ID NO:8. Nucleotide number 1-133 is the 5′-untranslated leader sequence. The coding sequence starts from 134 (ATG), and the stop codon (TAA) is at 1309-1312, encoding a polypeptide of 392 amino acids set forth in SEQ ID NO:9. There are 396 nucleotides in the 3′-untranslated region (1309-1714) including the poly(A) tail starting at nucleotide position 1661. There is no obvious polyadenylation signal in this region with the possible exception of a AT-rich region (1621-1630) located at 31 base upstream from the poly(A) tail.

The sequence of the cDNA insert in pCD520 (SEQ ID NO:8) was used as a query in a search of a DuPont EST database using BLAST programs and algorithms as search tools (Altschul, S. F. et al.(1990) J. Mol.Biol. 215:403-410 and Altschul, S. F. et al.(1997) Nucleic Acids Res. 25:3389-3402). An EST was identified by this method, and the full sequence of the cDNA clone from which it was derived is given in SEQ ID NO:10. The 5′-untranslated leader sequence is in nucleotide position from 1-101, the coding sequence starts from position 102, and ends with the stop codon (TAA) in position 1278-1280. This sequence also encodes a polypeptide of 392 amino acids the sequence of which is listed in SEQ ID NO: 11. The coding region of this second corn delta-9 desaturase gene shares significant homology with that listed in SEQ ID NO:8: The sequence share 63% identity and 83% similarity at the nucleotide level, and 77% identity at the amino acid level. There are 429 nucleotides in the 3′-untranslated region of SEQ ID NO: 10, including the poly(A) tail starting at nucleotide 1626. A putative polyadenylation signal (AATAA) is located at nucleotides 1588-1594.

### Example 4

### Spatial and Developmental Regulation of Delta-9 and Delta-12 Desaturases

Northern blot analyses were performed to investigate the spatial and developmental regulation of genes involved in lipid biosynthesis in corn embryos. Total RNA fractions were purified from leaves, sheath, tassels, roots and immature embryos dissected from the developing kernels at 15, 20, 25, and 30 days after pollination (DAP). RNA blots were prepared and hybridized individually with ³²P-labeled probes of corn fad2-1 (SEQ ID NO:1), delta-9 desaturase (SEQ ID NO:8), oleosin 16 kDa (Vance and Huang 1987), and globulin 1 (Belanger and Kriz, 1989, Plant Physiol. 91:636-643). The probes were prepared using gene-specific fragments purified as described below.

Using the sequence of fad2-1 (SEQ ID NO: 1), primers (SEQ ID NOS: 12 and 13) were designed to hybridize the 3′-end, and used in PCR with fad2-1 cDNA as the template.

| | |
|---|---|
| 5′-AGGACGCTACCGTAGGAA-3′ | SEQ ID NO:12 |
| 5′-GCGATGGCACTGCAGTA-3′ | SEQ ID NO:13 |

An expected 0.16 kb PCR fragment was gel-purified, and used as a fad2-1-specific probe. A cDNA clone containing the delta-9 desaturase (SEQ ID NO:8) was digested with EcoRI and XhoI, and a 1.7 kb fragment containing the entire cDNA insert was purified as the delta-9 desaturase gene probe.

The oleosin 16 kDa-specific probe was a 0.25 kb fragment purified from a PCR, using the corn embryo cDNA library as the template and primers (SEQ ID NO:14 and 15) hybridizing to the 3′-untranslated region of oleosin 16 kDa gene.

| | |
|---|---|
| 5′-CTTGAGAGAAGAACCACACTC-3′ | SEQ ID NO:14 |
| 5′-CTAGACATATCGAGCATGCTG-3′ | SEQ ID NO:15 |

A corn genomic clone containing the globulin-1 gene was digested by Xho I and Pst I. A 0.77 kb fragment containing the exon 4/intron 5/a portion of exon 5 was purified as the globulin-1 specific probe.

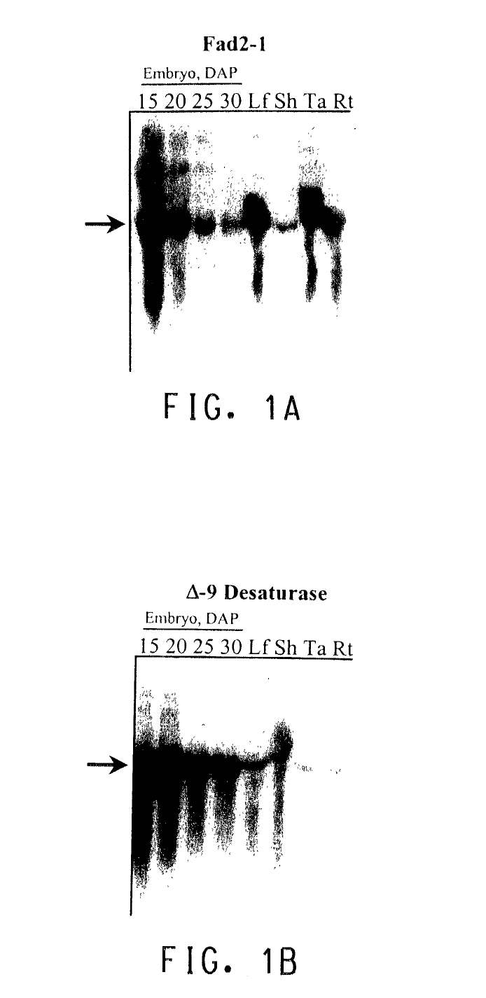

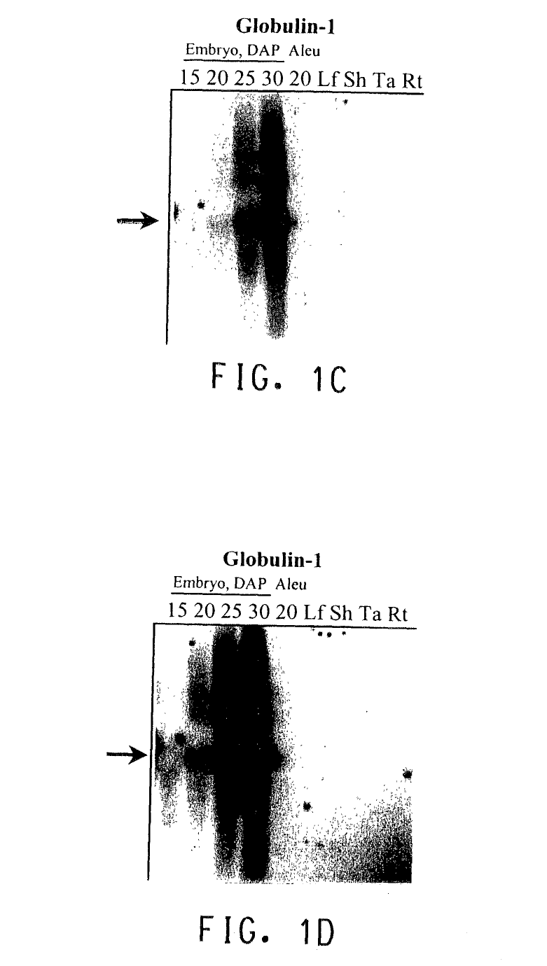

Analyses of the Northern blots are summarized in FIG. 1. Both the lipid biosynthetic genes (delta-9 and delta-12 desaturases) are expressed in all tissues/organs examined although at various levels. The expression of the desaturases seems coordinately regulated in embryos, but have different levels of expression spatially. The transcript homologous to the fad2-1 cDNAwas most abundant in the embryos at 15 DAP, and the message level declined toward maturation. The same developmental expression profile was detected for the delta-9 desaturase gene. There are high levels of expression of fad2-1 in both leaves and tassels, less in roots, and low but detectable in sheath. The delta-9 desaturase gene expressed at a lower level in these four tissues examined.

In order to down regulate the genes encoding the delta-9 desaturase, or the microsomal delta-12 desaturase, a seed-specific promoter which is expressed earlier than the target genes, or at least with timing that matches that of the target gene, would be highly desirable. Specifically, a promoter that is embryo/aleurone-specific is desired, since these are the tissues that store oil. The same promoter will be equally suitable for over-expression of a trait gene in the developing corn embryos. Therefore, there are two known maize genes which are good sources of promoter sequences, globulin-I (Belanger and Kriz, 1989, Plant Physiol., 91: 636-643) and oleosin 16 kDa (Vance and Huang, 1987, J. Biol. Chem. 262: 11275-11279). The expression profiles of these genes were also characterized by Northern blot analysis.

The steady state level of globulin-I transcripts began to accumulate at 20 DAP and reached a maximum level at a relatively late developmental stage (30 DAP). Although oleosin 16 kDa gene and globulin-1 are both tightly regulated spatially and are expressed only in seeds (Belanger and Kriz, 1989, Plant Physiol., 91: 636-643; Vance and Huang, 1988, J. Biol. Chem. 163; 1476-1481), the oleosin 16 kDa expression level is much higher judged by the strong hybridization signal in the embryo samples at all developmental stages (15-30 DAP) that were examined. The timing of oleosin 16 kDa expression is also much earlier than the globulin-1 gene. Immunofluoresent microscopy showed that oleosin 16 kDa protein is confined to the embryo and aleurone layer of developing seeds (Vance and Huang, 1988, J. Biol. Chem. 163; 1476-1481). Therefore, it was concluded that the oleosin 16 kDa promoter would be superior to globulin-I promoter for driving trait genes over-expression in corn embryos, and the timing of the expression would be optimal to down regulate the genes involved in lipid biosynthetic pathway.

### Example 5

### Isolation and Sequencing of a Corn Embryo and Aleurone-Specific Promoter

The profile of gene expression for oleosin 16 kDa was compared to the lipid biosynthetic genes and globulin-1, as shown in FIG. 1. It was concluded that oleosin 16 kDa is a very good source from which to isolate an embryo/aleurone specific promoter sequence.

Corn oleosin proteins contain three major structural domains; a largely hydrophilic domain at the N-terminus, a hydrophobic hairpin α-helical domain at the center, and an amphipathic α-helical domain at the C-terminus. However, oleosin 18 kDa and 16 kDa amino acid and nucleotide sequences are highly similar only at the central domain (Qu and Huang, 1990, J. Biol. Chem. 265: 2238-2243). Primers (SEQ ID NOS:16 and 17) were designed based on the published sequence of oleosin 18 kDa (accession # J05212, GenBank).

| | |
|---|---|
| 5′-AGGCGCTGACGGTGGCGACGCT-3′ | SEQ ID NO:16 |
| 5′-GTGTTGGCGAGGCACGTGAG-3′ | SEQ ID NO:17 |

These primers hybridize to the central domain region of the oleosin 18 kDa cDNA sequence. RT-PCR (Perkin-Elmer, Norwalk, Conn.) was performed using the total RNA purified from developing corn embryos and the above primer pairs to generate a unique 0.23 kb fragment. The fragment was gel purified, and ³²P-labeled as a probe to screen a corn genomic library (Missouri 17 line, in λFixII vector, Stratagene). Positive genomic clones were identified and recovered after three rounds of purification.

An oleosin 16 kda-specific oligomer (“3221-ATG”, SEQ ID NO:18) was synthesized.

5′-ACCTCCCGTCGCACCCCGGTGGTGATCAGCCATGGTAGGCTAGCAG-3′ SEQ ID NO: 18

This oligonucleotide contains a sequence complementary to the sequence flanking the translation start codon of oleosin 16 kDa gene. Specifically, the oligonucleotide is complementary to the region beginning 12 nucleotides prior to the translations start ATG and extending another 33 nucleotides into the coding region). This oligomer was labeled with ³²P using [γ-32P]ATP and T4 polynucleotide kinase (Life Technologies, Gaithersburg, Md.), and used to screen the positive genomic clones described above. One of the clones, λ3221, containing an insert of 15 kb, was identified as hybridizing strongly to the oligomer probe. DNA was purified from clone λ3221, digested with various restriction enzymes, electrophoresed on an agarose gel, and blotted onto a Zeta-probe nylon membrane (Stratagene). The same ³²P labeled oligomer (3221-ATG) was used as a probe to the X3221 restricted DNA blot to identify fragments containing the upstream sequences. Based on the hybridization signal patterns of various restriction digestion, and oleosin 16 kDa cDNA sequence, the λ3221 DNA was subcloned as the follows. The DNA of λ3221 was digested with Xho I and Xba I, and cloned into the pBluescript vector (pSK(−), Stratagene) previously cut by the same enzymes. The transformants were screened by the hybridization to the ³²P-labeled 3221-ATG oligomer. Positive clones were isolated. One of the clones (pBN164) was confirmed by sequencing to contain the elements of the upstream, 5′-leader, and the N-terminal part of the coding region of the oleosin 16 kDa gene.

The 1.7 kb sequence of the upstream region of oleosin 16 kDa gene in pBN164 is shown in SEQ ID NO:19. The transcription initiation site (+1) was identified at nucleotide position 1609 on the basis of primer extension data. This is 92 base pairs upstream of the ATG translation start codon. The putative TATA box (TATAAA) is located at position 1565-1571, 37-43 base pairs upstream of the transcription initiation site. Another TA-rich box is identified at position 1420-1426. These two TA-rich boxes are located in a region that is unusually GC-rich for an upstream element. The 5′-untranslated leader sequence is also GC-rich. There is a GC content of 67% from position 1326 to 1700, in contrast to a GC content of only 38% from position 1 to 1325. Southern blot analysis was conducted using genomic DNA purified from corn line LH192 (Holders Foundation Seeds, IA), hybridized with oleosin 16 kDa-specific probe. The result indicates that corn oleosin 16 kDa is encoded by one or two genes.

### Example 6

### Oleosin 16 kDa Promoter Deletion Assay

The relative activities of promoters from oleosin 16 kDa, and globulin-1, were analyzed using a transient expression assay. The 35S promoter of cauliflower mosaic virus was used as a positive control. The transient expression cassette used β-glucuronidase (GUS) as the reporter gene, fused with the 3′-end of the nopaline synthase gene (NOS) to provide a polyadenylation signal. The putative promoter fragment of olesoin 16 kDa contains the full-length (1.7 kb, SEQ ID NO: 19) of the upstream fragment of oleosin 16 kDa gene. The globulin-1 promoter contained a 1.1 kb upstream fragment from globulin-1 gene. The plasmid DNA was prepared according to the standard procedures (Wizard Miniprep kit, Promega, Madison, Wis.), coated onto gold particles, and bombarded into immature corn embryos dissected from cobs at 18-19 DAP. Nine embryos were placed onto each plate, and 3 plates were bombarded for every construct tested. After bombardment, the embryos were incubated at 37° in a substrate solution containing X-Gluc (Jefferson, 1989, Nature 342: 837-838) for 12 hours, and blue foci that developed indicating expression of the GUS gene were counted under the microscope. The result showed only minimal promoter activity was provided by the full-length upstream fragment of the oleosin 16 kDa gene, indicating there may be a negative regulatory element present in this region.

A number of oleosin 16 kDa promoters of varying length were designed to remove the potential negative regulatory element, and determine the optimal length with a high activity without losing its tissue specificity. Progressive deletions from the 5′- or 3′-end of this upstream sequence were made using PCR, or by restriction digests. The primers used in PCR, and the resulting putative promoter fragments, along with the corresponding nucleotide positions in SEQ ID NO:19 are shown in Table 1. The exon 1/intron 1 fragment (nucleotide position 1138-2220 in accession #X02382, GenBank) of maize shrunken-1 gene was cloned into the 5′-untranslated region as described below to further optimize the expression cassette.

TABLE 1
Putative promoter fragments from the oleosin 16 kDa gene.

| Promoter fragment | Primers used in | Nucleotide position | |
|---|---|---|---|
| (size in kb) | PCR^{a} | (as in SEQ ID1) | 5′-untranslated sequence |
| f168 (1.7)^{b} | | 1-1700 | Native oleosin 16 kDa |
| | | | 5′-leader^{c} |
| f184 (1.7)^{a} | u:I, d:J | 1-1700 | Sh1 |
| f222 (1.1) | u:A, d:E | 512-1619 | Sh1 |
| f220 (0.9) | u:B, d:E | 749-1619 | Sh1 |
| f218 (0.55) | u:C, d:E | 1075-1619 | Sh1 |
| f236 (0.4)^{b} | | 1254-1700 | Native oleosin 16 kDa |
| | | | 5′-leader |
| f254 (0.95) | u:B, d:H | 749-1700 | Native oleosin 16 kDa |
| | | | 5′-leader |
| f235 (1.4) | u:D, d:F | 99-1501 | Sh1 |
| 1231 (1.0) | u:A, d:F | 512-1501 | Sh1 |
| f232 (0.75) | u:B, d:F | 749-1501 | Sh1 |
| f233 (0.4) | u:C, d:F | 1075-1501 | Sh1 |
| f227 (1.2) | u:D, d:G | 99-1346 | Sh1 |
| f228 (0.8) | u:A, d:G | 512-1346 | Sh1 |
| f229 (0.6) | u:B, d:G | 749-1346 | Sh1 |
| f230 (0.3) | u:C, d:G | 1075-1346 | Sh1 |

a. PCR was conducted using the pBN164 plasmid DNA as the template, and upstream (u) and downstream (d) primers specified as indicated, except for f184, in which pBN168 was used as the template. A restriction enzyme recognition site (underlined) was built in most of the primers to facilitate the cloning.

(SEQ ID NO:20)

A: 5′-CTTATGTAATAGAAAAGACAGGATCCATATGG-3′

(SEQ ID NO:21)

B: 5′-GAGGAGTGAGGATCCTGATTGACTATCTCATTC-3′

(SEQ ID NO:22)

C: 5′-TCTGGACACCCTACCATTGGATCCTCTTCGGAG-3′

(SEQ ID NO:23)

D: 5′-AGAGTTGGATCCGTGTACAACTTGGTCTCTGG-3′

(SEQ ID NO:24)

E: 5′-GCCGCTGATGCTCGAGCTACGACTACGAGTGAGGTAG-3′

(SEQ ID NO:25)

F: 5′-ATGCGGGACTCGAGTCGGGGGCAGCGCGACAC-3′

(SEQ ID NO:26)

G: 5′-GTGGCGGGGCCGAATCTCGAGTGGGCCGTAGT-3′

(SEQ ID NO:27)

H: 5′-GCCACGTGCCATGGTAGGCTAGCAGAGCGAGCT-3′

(SEQ ID NO:28)

I: 5′-AACACACACCCATGGATATCACAG-3′

(SEQ ID NO:29)

J: 5′-GGTCTGACTTACGGGTGTC-3′

b. Fragment f168 was obtained by cutting pBN164 plasmid DNA with Xba I and NcoI. The fragment contain the full-length upstream region in pBN164. (A Nco I site is naturally present in the position of translation start codon in oleosin 16 kDa gene). Fragment f236 was present in pBN236. pBN236 was obtained by cutting pBN168 with Spe I and Xba I, blunt-end treated by Klenow enzyme, and religated.

c. The transcription initiation site (+1) is at nucleotide position 1609 in SEQ ID NO:19. Therefore, the 5′-leader sequence is considered from 1609-1700.

d. Sh1 includes the sequence of exon I/intron I (nucleotide position 1138-2220, in accession #X02382, GenBank) of maize shrunken-i gene.

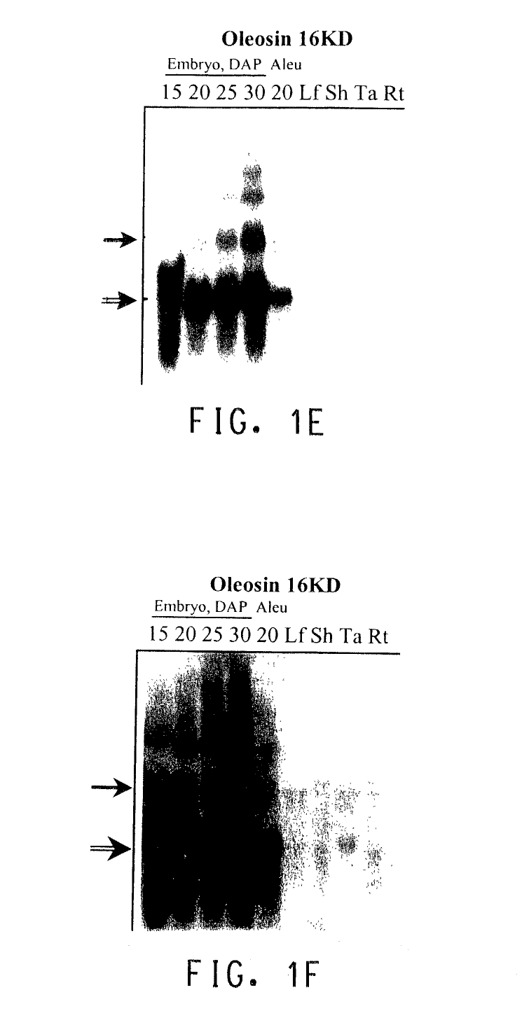

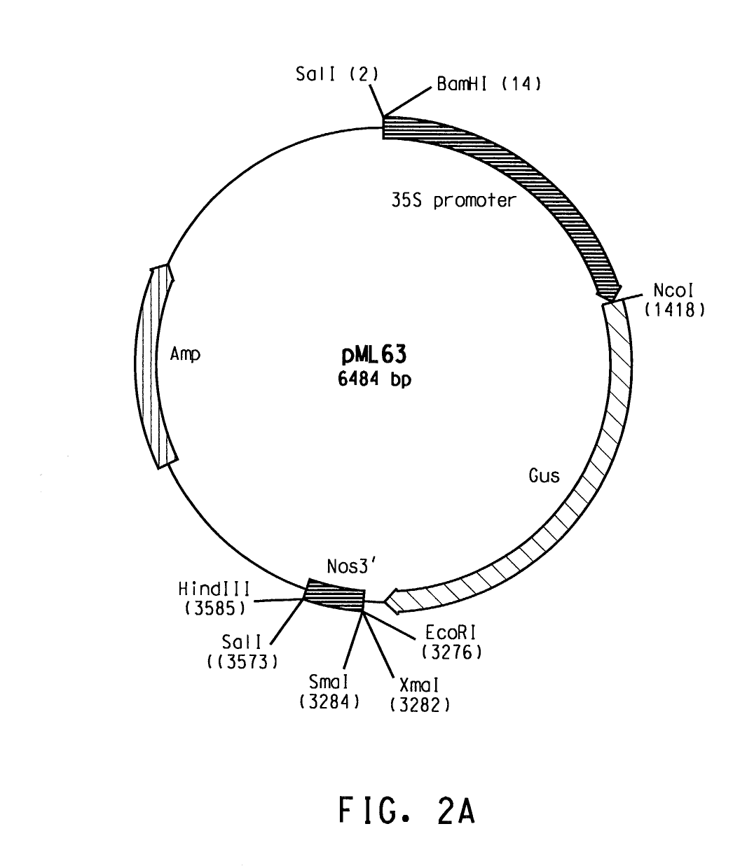

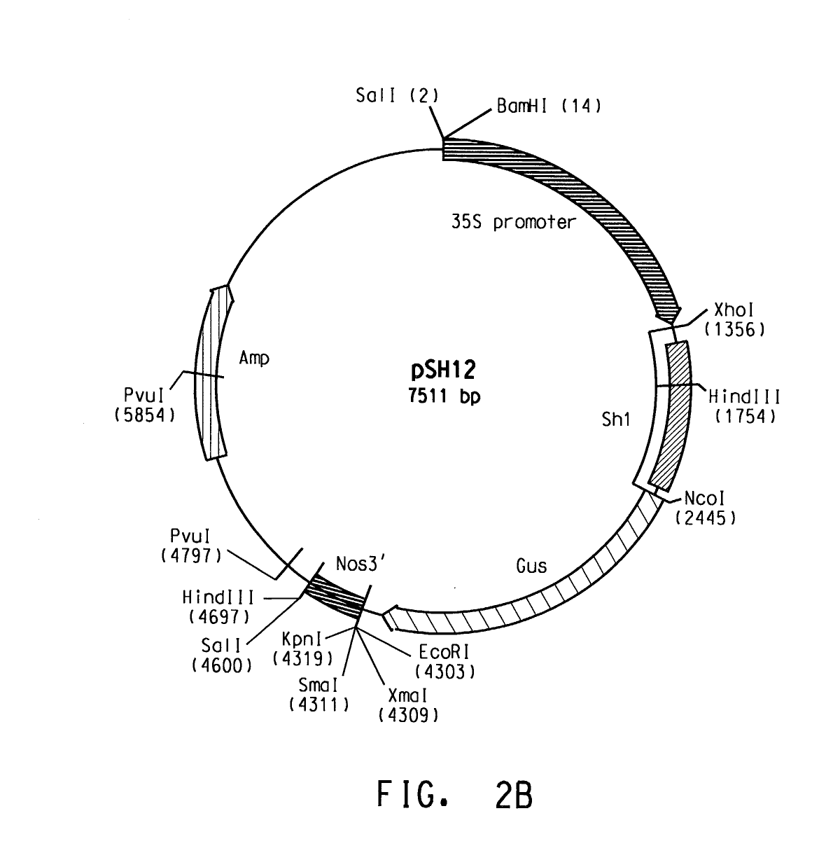

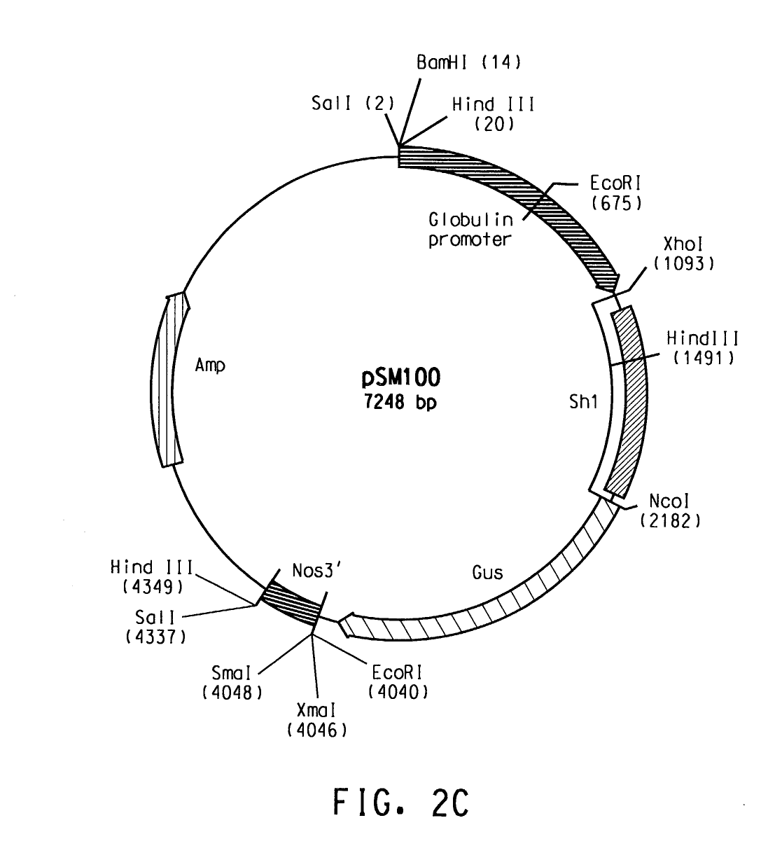

Three intermediate expression constructs, pML63, pSH12, and pSM100, were made. pML63 (FIG. 2A) was derived from the commercial available vector pGEM-9Zf(−) (Promega), with an insert containing the 35S promoter, the GUS coding region, and a NOS 3′-region. Plasmid pSH12 contains an exon 1/intron 1 fragment (Sh1) of corn shrunken-i gene, inserted in between the 35S promoter, and GUS coding region of pML63. The Shl fragment (nucleotide position of 1139-2230, in accession #X02382, GenBank) was obtained using a PCR approach. A pair of primers (SEQ ID NOS:30 and 31) were synthesized. The upstream primer (SEQ ID NO:30) contains an Xho I (underlined), and the downstream primer (SEQ ID NO:31) contains a Nco I site (underlined). These sequences were derived from the published sequence of maize sucrose synthase gene (X02382, GenBank) were used in PCR in which used DNA from a corn genomic library (Missouri 17 line, in λFixII vector, Stratagene) as the template.

| | |
|---|---|
| 5′-CTCTCCCGTCCTCGAGAAACCCTCC-3′ | SEQ ID NO:30 |
| 5′-CTTGGCAGCCATGGCTCGATGGTTC-3′ | SEQ ID NO:31 |

The resulting 1.1 kb fragment was gel-purified, digested with Xho I and Nco I enzymes, and inserted into the Xho I and Nco I site of pML63 to become pSH12 (FIG. 2B).

Plasmid pSM100 contains a globulin-1 promoter, Sh1 in the 5′-untranslated region, GUS gene, and a Nos 3′-end (FIG. 2C). The globulin-I promoter was obtained from a genomic clone isolated from a corn genomic library (constructed in EMBL3, Clontech, Palo Alto, Calif.) using end-labeled oligomers (SEQ ID NOS:32 and 33) as probes in the screening. The sequences of the oligomers are based on the globulin-I cDNA sequence available as GenBank accession M24845).

| | |
|---|---|
| 5′-ATGGTGAGCGCCAGAATCGTTGTCCTCCTC-3′ | SEQ ID NO:32 |
| 5′-CATCCTGGCGGTCACCATCCTCAGGAGCGT-3′ | SEQ ID NO:33 |

A positive clone with an insert about 10 kb hybridized to both the oligomer probes was confirmed to have the globulin-1 gene. A 0.45 kb fragment 5′ to the start codon was obtained from PCR using the 10 kb clone as the template. Primers used in the amplification of the 0.45 kb segment are presented in SEQ ID NOS:34 and 35. The upstream primer (SEQ ID NO:34) contains a site for the enzyme EcoRI (underlined), and the downstream primer contains a site for the enzyme NcoI (underlined).

| | |
|---|---|
| 5′-ATAGGGAATTCTCTGTTTTTCTAAAAAAAA-3′ | SEQ ID NO:34 |
| 5′-GCTCACCATGGTGTAGTGTCTGTCACTGTG-3′ | SEQ ID NO:35 |

The fragment was purified and cut with EcoRI and NcoI, inserted into a vector with comparable sites for cloning. A 0.66 kb Hind III—EcoRI fragment immediately upstream of the 0.45 kb region was cut out from the 10 kb clone and ligated upstream to the 0.45 kb fragment, giving rise to a final 1.1 kb globulin-1 promoter fragment. This clone was used in PCR with globulin-1 promoter-specific primers (SEQ ID NOS:36 and 37). The upstream primer (SEQ ID NO:36) contains a site for BamHI (underlined), and the downstream primer (SEQ ID NO:37) contains a site for XhoI (underlined).

SEQ ID NO:36

5′-GGGGGATCCAAGCTTGAGGAGACAGGAGATAAAAGT-3′

SEQ ID NO:37

5′-GGGCTGCAGCTCGAGGGTGTAGTGTCTGTCACTGTGATA-3′

The resulting 1.1 kb PCR fragment was purified, digested with BamHI and XhoI, and inserted into the BamHI and XhoI sites of pSH12 to replace the 35S promoter. The resulting plasmid is designated as pSM100 (FIG. 2C).

All putative oleosin 16 kDa promoter fragments (listed in Table 1) were gel-purified before cloning into the expression vector. The f168 fragment was inserted into the XbaI and NcoI site of pML63 (to replace the original 35S promoter in the construct), and the new construct was named pBN168.

The purified PCR fragments described in Table 1 were digested with the corresponding restriction enzymes designed into the primers (BamHI and XhoI for f222, f220, f218, f235, f231, f232, f233, f227, f228, f229, and f230), and inserted into the expression vector (pSM 100) previously digested by the same enzymes in order to replace the globulin-I promoter. Fragment f184 was cut with Nco I, and inserted into the NcoI site of pBNi68. The resulting construct, pBNi84, contained the native oleosin 16 kDa 5′-leader sequence with the Shl element in the 5′-untranslated region. Fragment f254 was digested with BamHI and NcoI, and inserted into the BamHI/NcoI site of pML63.

The different promoters and 5′-untranslated fragments contained in these constructs are listed in Tables 1 and 2. The sequences of each of these promoters (as derived from the full length 1.7 kb promoter, and not including the restriction sites introduced during the cloning) are set forth in the sequence listings, as follows. SEQ ID NO:38 is the 1.1 kb promoter fragment, SEQ ID NO:39 is the 0.9 kb promoter fragment, SEQ ID NO:40 is the 0.55 kb promoter fragment, SEQ ID NO:41 is the 0.95 kb promoter fragment, SEQ ID NO:42 is the 1.4 kb promoter fragment, SEQ ID NO:43 is the 1.0 kb promoter fragment, SEQ ID NO:44 is the 0.75 kb promoter fragment, SEQ ID NO:45 is the 0.4 kb promoter fragment, SEQ ID NO:46 is the 1.3 kb promoter fragment, SEQ ID NO:47 is the 0.8 kb promoter fragment, SEQ ID NO:48 is the 0.6 kb promoter fragment, SEQ ID NO:38 is the 1.1 kb promoter fragment, and SEQ ID NO:49 is the 0.3 kb promoter fragment.

Purified plasmid DNAs from these constructs were used in the transient expression assays as described previously. GUS staining assay results indicating promoter activities are summarized in Table 2.

TABLE 2
Oleosin 16 kDa promoter deletion assay.

| Plasmid | Construct | Promoter activity^{a} |
|---|---|---|
| pBN168 | pOle-1.7 kb5′::GUS::Nos3′ | +/− |
| pBN184 | pOle-1.7 kb5′::Sh::GUS::Nos3′ | − |
| pBN222 | pOle-1.1 kb5′::Sh::GUS::Nos3′ | +++ |
| pBN220 | pOle-0.9 kb5′::Sh::GUS::Nos3′ | +++++ |
| pBN218 | pOle-0.55 kb5′::Sh::GUS::Nos3′ | ++++ |
| pBN254 | pOle-0.95 kb5′::GUS::Nos3′ | + |
| pBN236 | pOle-0.4 kb5′::GUS::Nos3′ | +/− |
| pBN235 | pOle-1.4 kb5″::Sh::GUS::Nos3′ | ++ |
| pBN231 | pOle-1.0 kb5″::Sh::GUS::Nos3′ | ++ |
| pBN232 | pOle-0.75 kb5″::Sh::GUS::Nos3′ | ++ |
| pBN233 | pOle-0.4 kb5″::Sh::GUS::Nos3′ | ++ |
| pBN227 | pOle-1.3 kb5′′′::Sh::GUS::Nos3′ | + |
| pBN228 | pOle-0.8 kb5′′′::Sh::GUS::Nos3′ | + |
| pBN229 | pOle-0.6 kb5′′′::Sh::GUS::Nos3′ | + |
| pBN230 | pOle-0.3 kb5′′′::Sh::GUS::Nos3′ | + |
| pSM100 | pGlo-1.1 kb5′::Sh::GUS::Nos3′ | ++ |

^{a}Promoter activity was measured by a transient expression assay of the reporter gene, GUS. The + was assigned based on the visual estimation of the intensity and counts of the blue foci. −: 0, +/−: 0-1; +: 2-10; ++:10-50; +++: 50-100; ++++: 50-100, but significantly darker blue than +++; +++++: >150 blue foci.

The full-length promoter (as contained in pBN 168 and pBN 184), whether or not in conjunction with the Shl intron element, confers non-detectable or minimal promoter activity in the transient expression system. Promoter activity was increased when this region was progressively deleted from the far upstream end. It appears that there is a negative-regulatory element in this far upstream region (1-511). Deletion of this region as in pBN222 significantly increased the GUS expression as compared to the activity of pBN184 in the assay. Removal of yet more sequence, up to nucleotide position 748, further enhanced the activity of the promoter, as was demonstrated with construct pBN220. However, promoter activity decreased if the upstream sequence was deleted beyond position 748 (pBN218 vs. pBN220).

Inclusion of the TATA box (1566-1571) is important for attaining high promoter activity. However, the upstream TATA-rich element (1420-1436) can substituted for the TATA box (1566-1571), albeit with a significantly lower activity. The function of the GC-rich region (1326-1700) surrounding the TATA boxes is not apparent from these data. Minimal promoter activities was still detected when the entire GC-rich region, including both the TATA boxes, was deleted.

Intron enhancement is very important in optimizing gene expression. None of the constructs lacking the Sh1 element provided any significant level of GUS expression in the assay. The oleosin 16 kDa promoter with an optimized length and composition, as in pBN220, was found to be stronger than the globulin-1 promoter (as contained in pSM100). The results of the Northern blot analyses characterizing early timing of expression in the young developing corn embryos, combined with the demonstration of its high activity in the expression assay, indicated that the optimal embryo/aleurone-specific promoter is the 0.9 kb fragment (SEQ ID NO:39) isolated from the oleosin 16 kDa gene combined with a Shl exon 1/intron 1 element in the 5′-untranslated region.

### Example 7

### Corn Embryo/Aleurone-Specific Expression Constructs with Lipid Trait Genes

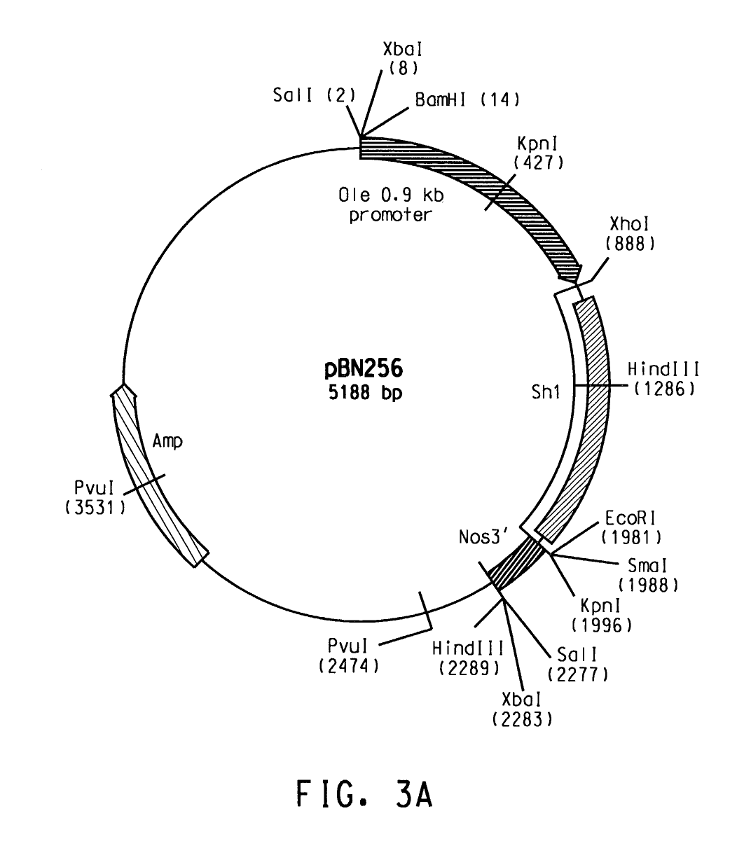

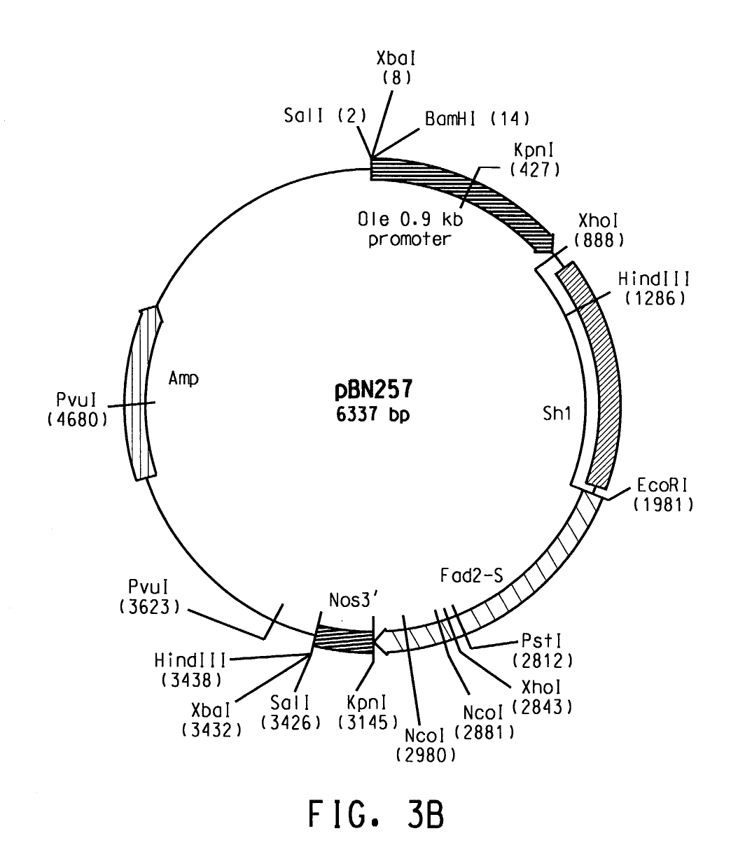

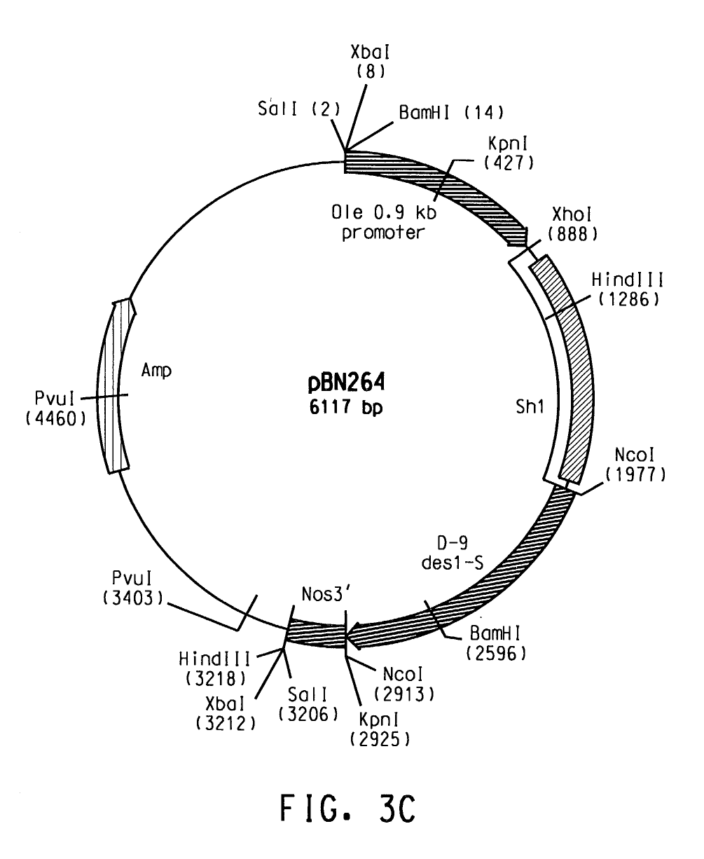

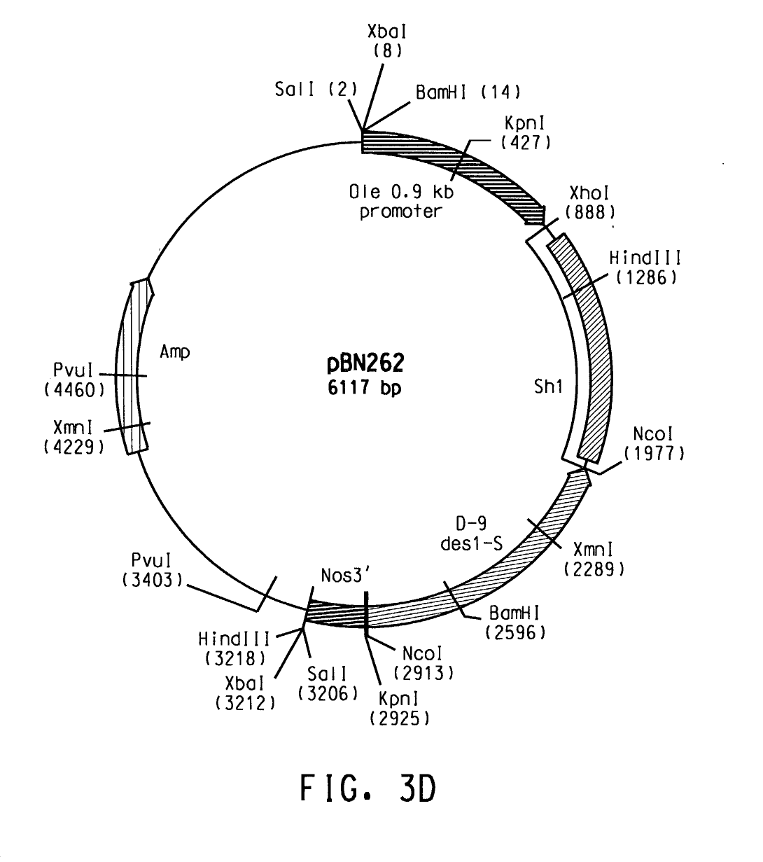

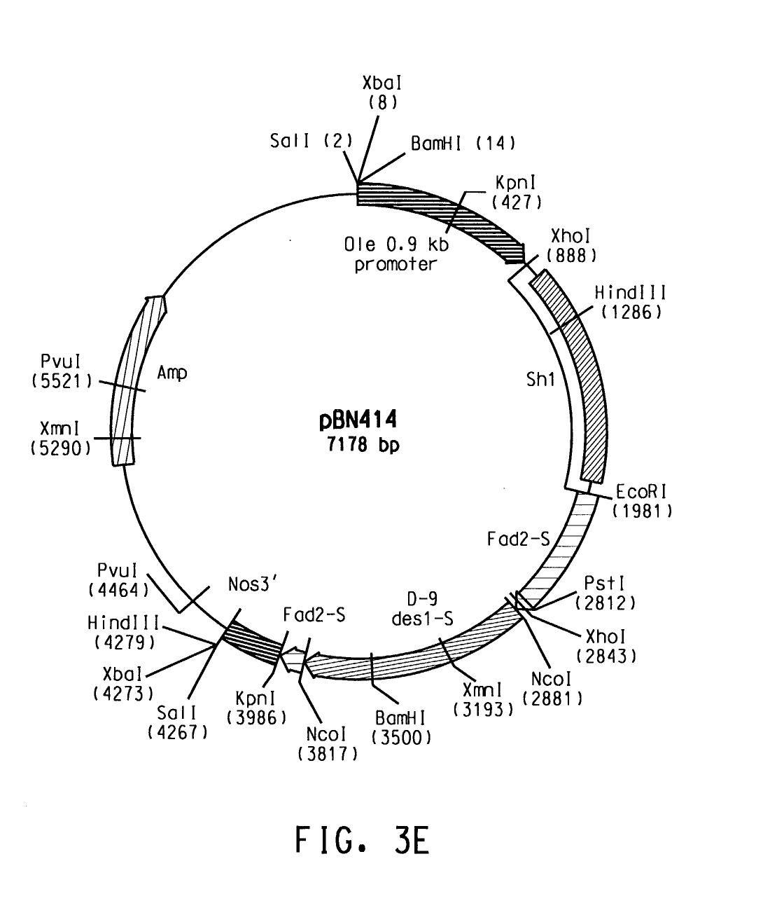

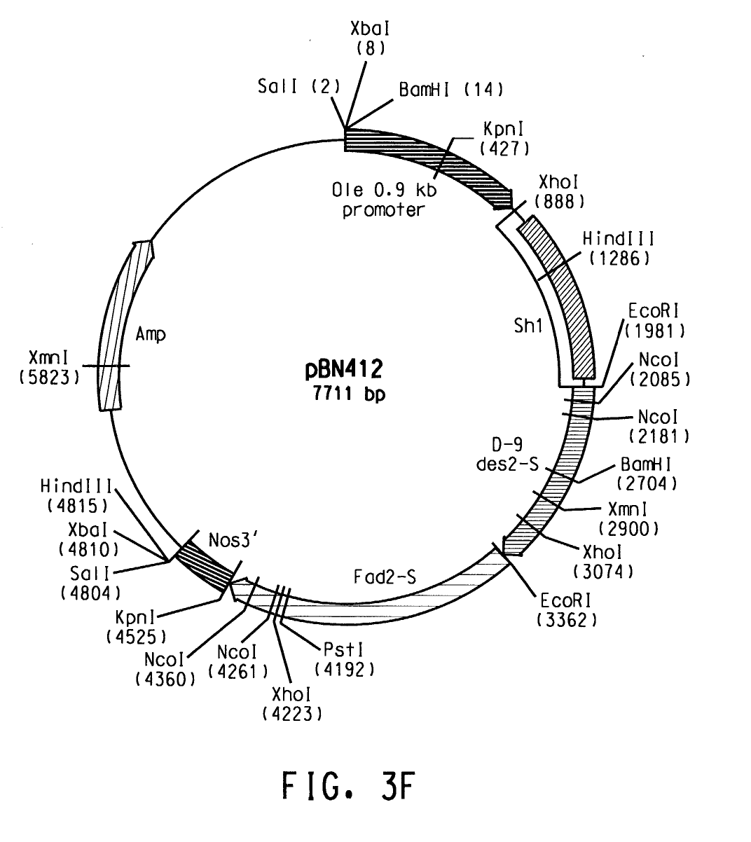

Expression constructs comprising a maize oleosin 16 kDa promoter (0.9 kb in length, Table 1 and 2, and SEQ ID NO:39), an intronl/exonl element (1.1 kb) from the shrunken-i gene located between (3′ to) the promoter and (5′ to) the cDNA fragment, a cDNA fragment encoding a portion of the trait gene in either sense or antisense orientation with respect to the promoter, and a Nos 3′-end located 3′ to the cDNA fragment, were constructed and used in corn transformation to alter the level of the enzyme encoded by the trait gene in corn grains (FIGS. 3B-3F). The construct design is suitable to express any target trait gene not mentioned in this patent in a corn embryo/aleurone-specific manner. The selectable marker on the vector backbone may be any antibiotic (e.g., ampicillin, hygromycin, kanamycin) resistant gene.

An intermediate construct, pBN256, modified from pBN220 was made as the starting vector for the various expression constructs with lipid trait genes. pBN220 was digested with NcoI and EcoRI to delete the GUS coding sequence, end-filled with dNTPs and Klenow fragment of DNA polymerase I, and re-ligated. The resulting plasmid was designated pBN256 (FIG. 3A).

PCR was used to obtain a fragment containing the fad2-1 coding region with Kpn I restriction site at both ends. The fad2-1 cDNA clone was used as the template with primers (SEQ ID NOS:50 and 51) specific to the fad2-1 sequence each containing a site for KpnI (underlined).

| | |
|---|---|
| 5′-CGGGGTACCGATGACCGAGAAGGAGCGGG-3′ | SEQ ID NOS:50 |
| 5′-GGCGGTACCTAGAACTTCTTGTTGTACCA-3′ | SEQ ID NOS:51 |

The expected 1.2 kb fragment was gel-purified, digested with Kpn I, and cloned into a vector with a comparative Kpn I site to facilitate propogation and further manipulation. The Kpn I fragment was digested out from this new construct, and the ends were blunted as above, inserted into the Sma I site of pBN256, to become pBN257. This clone contains a near full-length of fad2-1 coding region, but the ATG translation start codon is out of frame (FIG. 3B).

A DNA fragment containing the delta-9 desaturase coding region was recovered by PCR using the delta-9 desaturase cDNA clone (SEQ ID NO:8) DNA as the template and coding region-specific primers (SEQ ID NOS:52 and 53) that contained NcoI sites. The resulting fragment was gel purified, cut by Nco I, and inserted into the Nco I site of the modified pBN220 in which the GUS gene had been previously removed.

| | |
|---|---|
| 5′-GGCCTCCGCCATGGCGCTCCGCTCCACGACG-3′ | SEQ ID NOS:52 |
| 5′-CTCCAACTCAAGCAGTCGCCATGGGTTTCC-3′) | SEQ ID NOS:53 |

(Plasmid pBN220 was cut by Nco I and Sma I to remove the GUS gene, end-filled in by Klenow treatment, and religated as the modified GUS-free vector.) The resulting clones contained a truncated corn delta-9 desaturase coding region (approximately 0.9 kb, comprising 79% of the full-length coding sequence) in each of the two possible orientations, sense (pBN264, FIG. 3C) and antisense (pBN262, FIG. 3D).

The 0.9 kb Nco I fragment of the delta-9 desaturase gene(SEQ ID NO:8) was also cloned into the Nco I site of pBN257 to create a construct, pBN414, containing a fused trait gene of fad2-1 and delta-9 desaturase, both in the sense orientation, as shown in FIG. 3E.

The coding sequence of fad2-1 in pBN414 is out of frame as in pBN257, and its C-terminal sequence was interrupted by the insertion of the delta-9 desaturase fragment (79% of the full length coding region shown in SEQ ID NO:8).

The second delta-9 desaturase clone (SEQ ID NO: 10) was cut by EcoRI, and the 1.1 kb EcoRI fragment was purified and inserted into the EcoRI site of pBN257 to create a new construct, pBN412 (FIG. 3F), containing a fused trait gene of delta-9 desaturase and fad2, both in sense orientation. In pBN412, the delta-9 desaturase fragment contains a full-length coding region (SEQ ID NO: 10). The translation start codon ATG for the delta-9 desaturase is in frame in pBN412, but fad2 coding sequence is out of frame.

### Example 8

### Transgenic Corn

a. Corn Transformation

The chimeric genes described above can be introduced into corn cells by the following procedure. Immature corn embryos are dissected from developing caryopses derived from crosses of the inbred corn lines H99 and LH132, or from crosses of the inbred corn lines H99 and LH195, or a public High II line (Armstrong, 1991, Maize Genetics Co. News Letter 65:92-93), or any corn lines which are transformable and regenerable. The embryos are isolated 10 to 11 days after pollination when they are 1.0 to 1.5 mm long. The embryos are then placed with the axis-side facing down and in contact with agarose-solidified N6 medium (Chu et al., (1975) Sci. Sin. Peking 18:659-668). The embryos are kept in the dark at 27°. Friable embryogenic callus proliferates from the scutellum of these immature embryos. It consists of undifferentiated masses of cells with somatic proembryoids and embryoids borne on suspensor structures. The embryogenic callus isolated from the primary explant can be cultured on N6 medium and sub-cultured on this medium every 2 to 3 weeks. The plasmid, p35S/Ac (obtained from Dr. Peter Eckes, Hoechst Ag, Frankfurt, Germany) may be used in transformation experiments along with the trait gene (co-bombardment) in order to provide for a selectable marker. This plasmid contains the Pat gene (see European Patent Publication 0 242 236) which encodes phosphinothricin acetyl transferase (PAT). This gene is from Streptomyces viridochromogenes, and its sequence is found as GenBank accession X65195. The enzyme PAT confers resistance to herbicidal glutamine synthetase inhibitors such as phosphinothricin (also available as the compound designated gluphosinate). Thepat gene in p35S/Ac is under the control of the 35S promoter from Cauliflower Mosaic Virus (Odell et al. (1985) Nature 313:810-812) and the 3′ region of the nopaline synthase gene (NOS 3′-end) from the T-DNA of the Ti plasmid of Agrobacterium tumefaciens. Alternatively, the gel-purified pat gene fragment, including the 35 S promoter, pat gene coding region, and the NOS 3′-end, may be used as the selectable marker. It will be appreciated by the skilled worker that the fragment used to provide selection in transformations can vary considerably, and that any fragment containing the 35S promoter operably linked to the pat gene is capable of providing the desired selectable trait. Another gene that is useful as a selectable marker for resistance to phosphinothricin, and which may be provided on a plasmid or as a separate DNA fragment, is the bar gene from Streptomyces hygroscopicus (GenBank accession X17220).

The particle bombardment method (Klein et al., (1987) Nature 327:70-73) was used to transfer genes to the callus culture cells. According to this method, gold particles (0.6 μm or 1 μm in diameter) were coated with DNA using the following technique. Approximately 10 μg of plasmid DNAs were added to 50 μL of a suspension of gold particles (60 mg per mL). Calcium chloride (50 μL of a 2.5 M solution) and spermidine free base (20 μL of a 1.0 M solution) were added to the particles. The suspension was vortexed during the addition of these solutions. After 10 minutes, the tubes were briefly centrifuged (5 sec at 15,000 rpm) and the supernatant was removed. The particles were resuspended in 200 μL of absolute ethanol, centrifuged again and the supernatant was removed. The ethanol rinse was performed again and the particles were resuspended in a final volume of 30 μL of ethanol. An aliquot (5 μL) of the DNA-coated gold particles was then placed in the center of a Kaptona flying disc (Bio-Rad Labs). The embryogenic tissue was placed on filter paper over agarose-solidified N6 medium. The tissue was arranged as a thin lawn that covered a circular area of about 5 cm in diameter. The petri dish containing the tissue was placed in the chamber of the PDS-1000/He approximately 8 cm from the stopping screen. The air in the chamber was evacuated to a vacuum of 28 inches of Hg. The DNA-coated particles were accelerated into the corn tissue with a Biolisticä PDS-1000/He (Bio-Rad Instruments, Hercules, Calif.), using a helium pressure of 1000 psi, a gap distance of 0.5 cm and a flying distance of 1.0 cm.

Seven days after bombardment the tissue was transferred to N6 medium that contained gluphosinate (5 mg per liter) and lacked casein or proline. The tissue continued to grow slowly on this medium. After an additional 2 weeks the tissue was transferred to fresh N6 medium containing gluphosinate (selection medium). The tissue was cultured on the selection medium and was transferred every 2 weeks for a total 3-4 passages. Areas of about 1 cm in diameter of actively growing callus were identified on some of the plates containing the selection medium. These calli continued to grow when sub-cultured on the selective medium.

Plants were regenerated from the transgenic callus by first transferring clusters of tissue to N6 medium supplemented with 0.2 mg per liter of 2,4-D (regeneration medium). After 2-3 weeks the tissues began to form somatic embryo-like structures and showed green areas when the tissues are transferred and grown under light. Plantlets emerged after a total of 3-4 weeks on regeneration medium, and were transferred individually into plant tissue culture vessels containing the regeneration medium. After sufficient growth of root and shoot, the plantlets were transplanted to 4 inches pots in the growth chamber, and later re-potted into 10-12 inches pots, and grown to maturity in the greenhouse (Fromm et al., (1990) Bio/Technology 8:833-839).

b. Transgenic Corn with High Saturate Fatty Acid Composition in the Grain

Using biolistic gun method described above, corn callus was co-bombarded with pBN262 plasmid DNA, and the bar gene fragment. Stable transformants were selected according to procedures described above, and transgenic corn plants were regenerated. Primary transformants (designated as R0 plants) were grown in the greenhouse. The plants were either selfed or crossed using wild type pollen from Holdens line LH132. The cobs were harvested at 30 DAP. Embryos were dissected out of kernels, and sterilized. Small pieces of scutella were taken from each individual embryo and used for fatty acid composition assays by the GC method as described in WO 94/11516. The remaining embryos were planted in tissue culture vessels containing the regeneration medium. Embryos with a positive phenotype (i.e., a high level of saturated fatty acids in the lipid fraction) were transplanted from the culture vessels in pots, and grown into R1 plants in the greenhouse. The mature R1 plants were either selfed or crossed with the wild type pollen (from line 5-12-24, Pioneer Hybrid International, Johnston, Iowa). The cobs were harvested at 45 DAP, and R2 kernels were collected. Small piece of scutella were taken from individual kernels, and used for analyses of their fatty acids.

Two independent transgenic lines were identified as having a high saturated fatty acid phenotype, FA01 3-2-4 and FAO1 3-3-2.

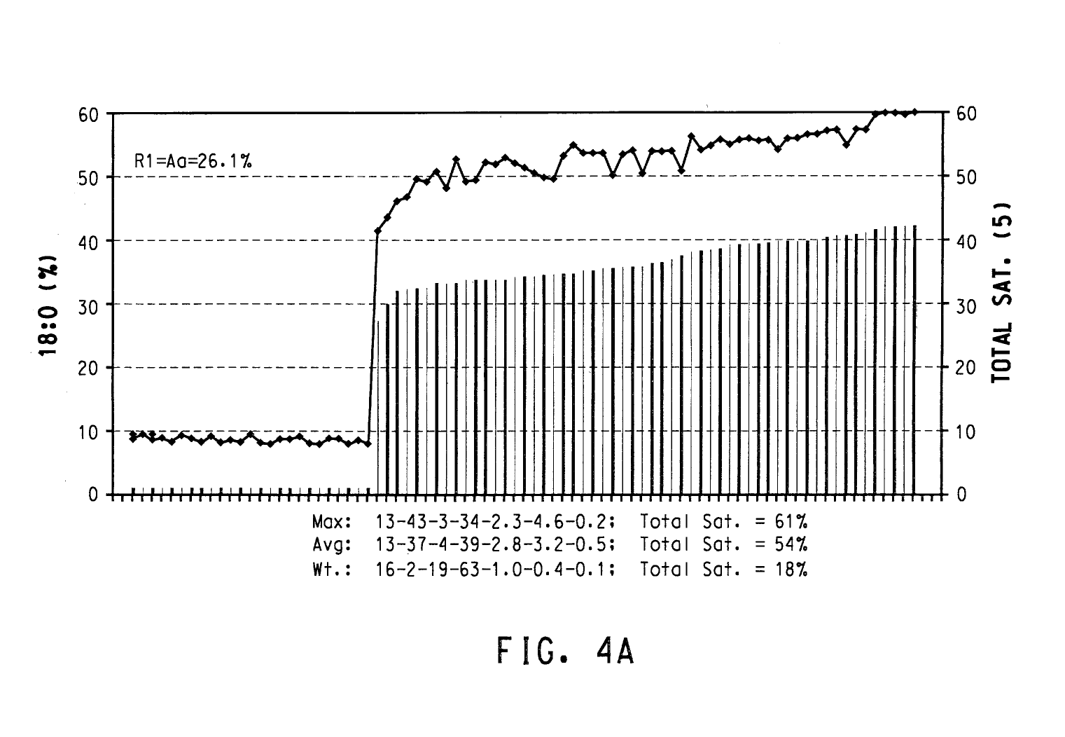

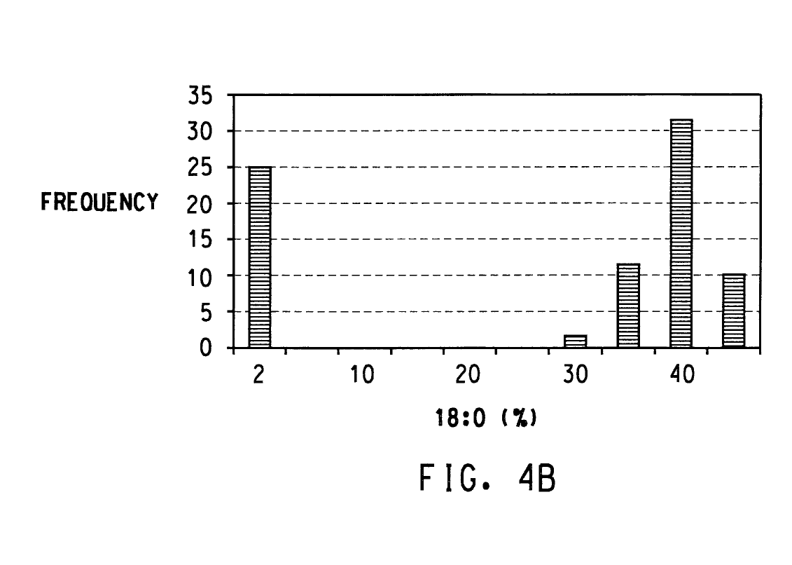

FIG. 4A shows a typical example of the phenotype of R1:2 kernel segregants from a single cob harvested from a R1 plant of line FA013-2-4. The R0 generation of this plant was cross-pollinated with wild-type pollen from LH132 (Holder). The cob was harvested and lipid composition of single kernels analyzed. The results shows a 1:1 (high saturate phenotype: wild type) seed segregation indicating the presence of a single transgene insertion locus in FA013-2-4. A heterozygous kernel that contained 26.1% of stearic acid (vs. wild type as 2%) was planted and grown into a R1 plant. The R1 plant was selfed, and the data from analyses of the R2 seeds indicated a segregation ratio as 3:1 (FIGS. 4A and 4B), confirming that FA013-2-4 contains a single locus of transgene insertion, and that the trait phenotype is dominant. In the R2 seed segregants, the stearate content in the kernels ranged from 27-43%, and the average fatty acid composition was 13% 16:0, 37% 18:0, 4% 18:1, 39% 18:2, 2.8% 18:3, and 0.5% 20:0 and 20:1. The total saturate fatty acid content was 54%. The maximum saturated fatty acid content was found to be as high as 61%. This was in a line that had an overall composition of 13% 16:0, 43% 18:0, 3% 18:1, 34% 18:2, 2.3% 18:3, 4.6% 20:0, and 0.2% 20:1. This is compared to the composition of the wild-type segregants profile of 16% 16:0, 2% 18:0, 19% 18:1, 63% 18:2, 1.0% 18:3, and 0.1% 20:0. The wild-type segregants had a total saturated fatty acid content of 18%.

The germination rate of seed from line FA013-2-4 is close to 100% in standard growth chamber conditions, indicating that the saturated fatty acid content in embryo/aleurone does not affect the seed viability.

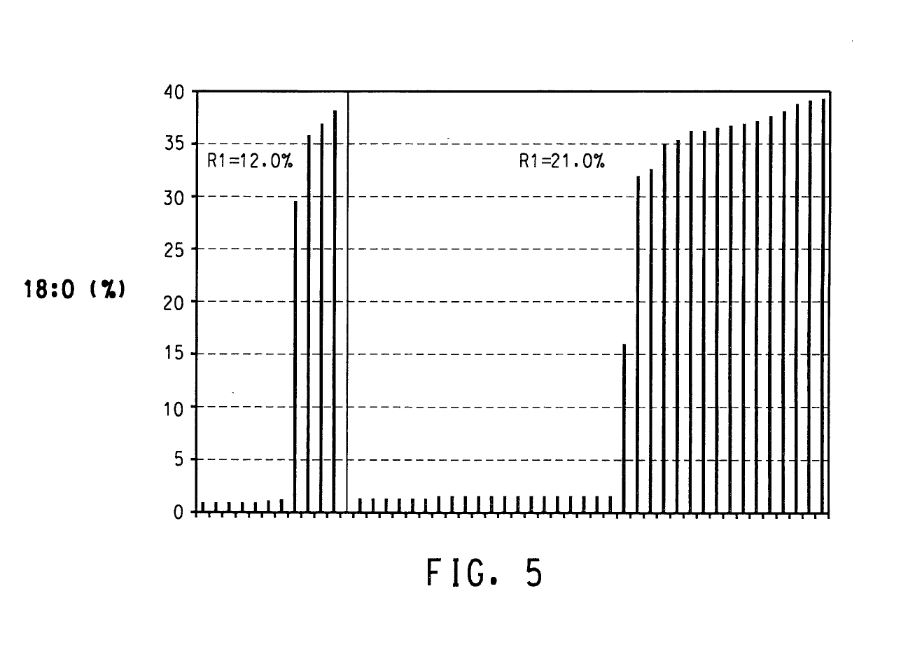

FIG. 5 shows a typical example of the phenotype and segregation of R1:2 kernels harvested from two R1 plants of line FA013-3-2-15. Their respective RO plant was selfed, and the corresponding R1 plants were both cross-pollinated with the wild type pollen from line 5-12-24. The first plant was derived from a R0:1 kernel originally containing 12% stearate, and the second plant from a kernel with 21% stearate content. However, the maximal stearate content of R1:2 kernels from both plants reaches up to 38-39%. The range of variation in the R1:2 kernels stearate levels was 29-38%, and 16-39%, respectively. This indicated the presence of a single transgene insertion locus in line FA013-3-2-15 based on the segregation ratio. The average total saturate content was more than 50%, and the seed germination rate for this line was about 40%.

R3:4 seeds were obtained from homozygous plant of FA013-2-4 event. The lipid composition of the homozygous grains was, on average, 15% 16:0, 15% 18:0, 14% 18:1, 53% 18:2, 1.5% 18:3, 1.5% 20:0, and 0.5% 20:1. However, kernels harvested from a heterozygous plant at the same R3 :4 generation contains a higher stearate content (31% versus the 15% from the homozygous background). A similar result was obtained in the grains harvested from the crossing using this heterozygous plant as the pollen donor onto a hybrid female plant (34K77, DuPont) in the TopCross® (TC) grain production method (Table 3).

TABLE 3
Kernel lipid composition in RO:1, homozygous and heterozygous
R3:4, and various crossing of FA013-2-4.

| | Phenotype (%) | | | | | | |
|---|---|---|---|---|---|---|---|
| Genotype | 16:0 | 18:0 | 18:1 | 18:2 | 18:3 | 20:0 | 20:1 |
| R0:1 × LH132 | 14 | 23 | 12 | 47 | 4 | | |
| R3:4 selfed (homozygous) | 15 | 15 | 14 | 53 | 1.5 | 1.5 | 0.5 |
| R3:4 selfed(heterozygous)^{a} | 12 | 31 | 8 | 44 | 2.4 | 3.0 | 0.3 |
| 34K77 (TC) × R3^{a} | 12 | 32 | 7 | 45 | 2.1 | 2.7 | 0.3 |
| WT^{b} | 15 | 1.2 | 18 | 65 | 0.7 | 0.3 | 0.3 |

^{a}The data represent the average lipid composition from kemels with the positive phenotype. The kernels of R3:4 were from the selfed cob of the heterozygous R3 plant. The same R3 plant was used as the pollen donor to pollinate 34K77 plants.
^{b}A few 34K77 plants were selfed to obtained the wild-type kernels as the control.

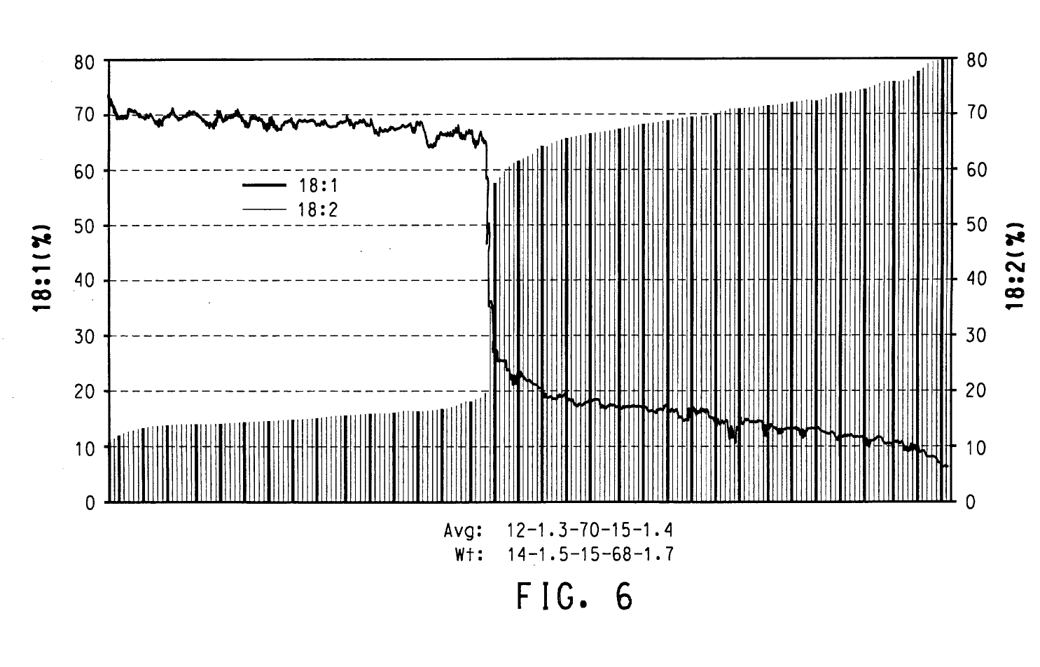

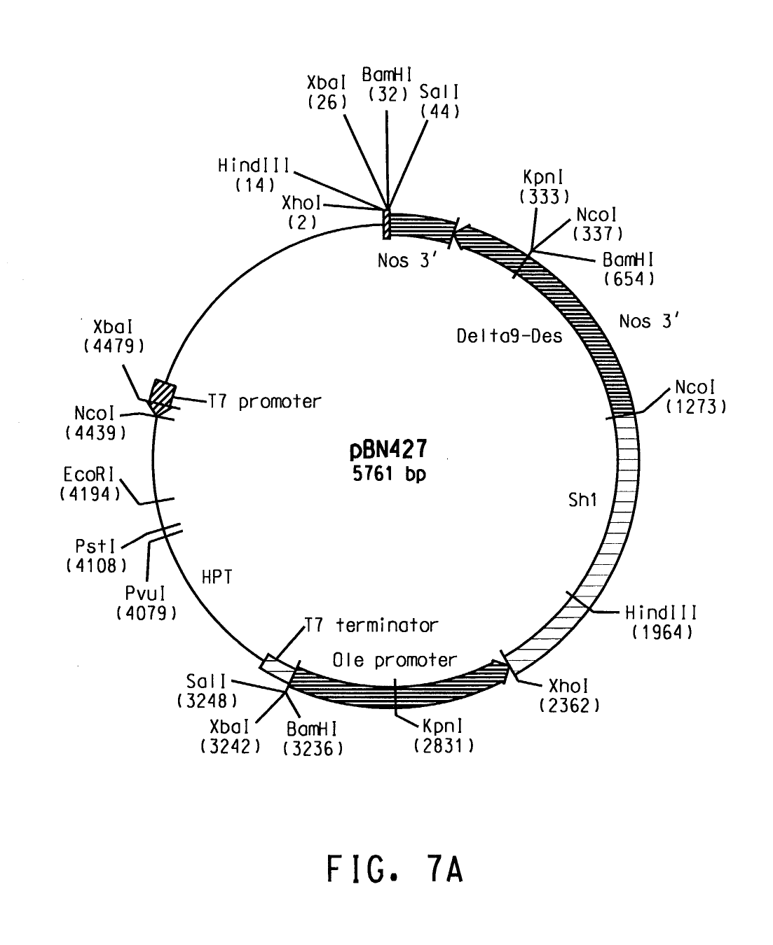

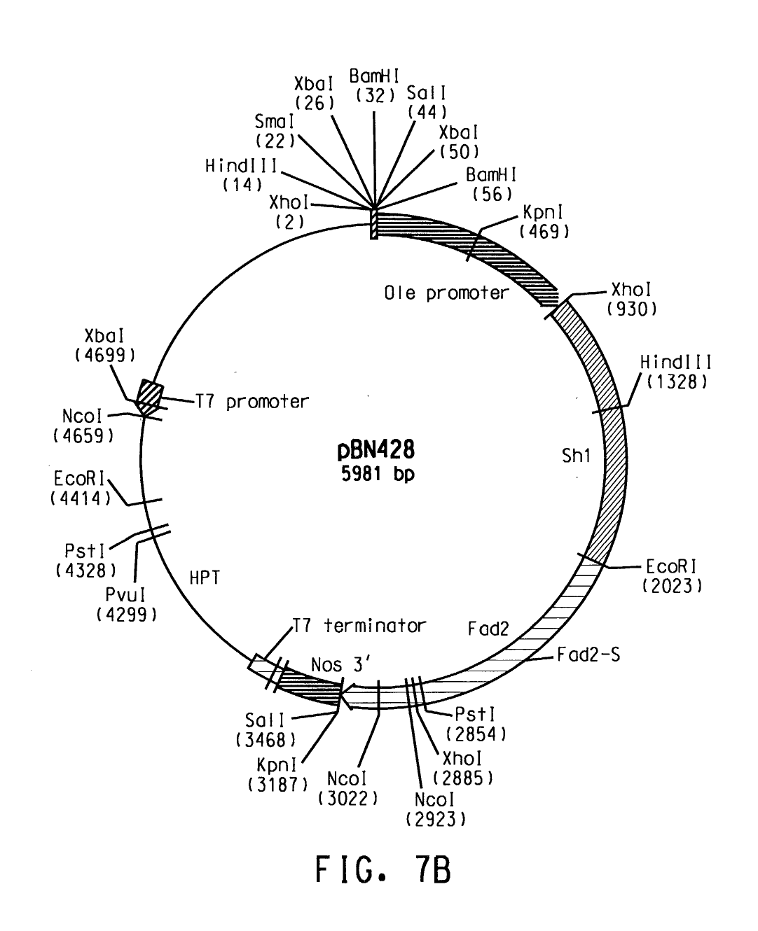

Using processes similar to those described above, new transgenic events with high stearate—and hence high saturate—phenotypes were generated (Table 4). The trait gene constructs used in these experiments are from either pBN264 or pBN427 (FIG. 7A). Plasmid pBN264 is similar to the pBN262, except that the delta-9 desaturase is in a sense orientation relative to the promoter. The transgene sequence is contained within a Sal I fragment (position 3248-44) of pBN427 and is identical to the corresponding Sal I fragment of pBN264 (position 2-3206). However, pBN427 uses a vector backbone with a hygromycin resistance selectable marker (HPT, from pKS 17, described in WO 94/11516), versus the ampicillin marker in pBN262 and pBN264. The transgene prepared for the bombardment were either the restriction enzyme digested and agarose gel purified DNA fragment from pBN264 (for events derived from the FA025 experiment, the transgene fragment was marked as fBN264), or the intact pBN427 plasmid DNA (for events derived from the FA029 experiment). The restriction enzyme used to cut out the transgene may be Sal I or Xba I, which release a transcriptionally functional transgene fragment of 3.2 kb, which can then be purified following agarose gel electrophoresis. The use of a transgene DNA fragment, rather than the entire plasmid, allows the recovery of transgenic events which do not contain a bacterial antibiotic resistance gene.

TABLE 4
Transgenic events with high stearate phenotype

| | Transgenic events | Stearate^{a} | Total Sat.^{b} | Construct^{c} | Co-supp. freq.^{d} |
|---|---|---|---|---|---|
| | Wild-type | <2% | 18% | | |
| 1) | FA025-1-4 | 16-27% | 32-42% | fBN264 | |
| 2) | FA025-2-1 | 12-39% | 28-60% | fBN264 | |
| 3) | FA025-2-12 | 17-39% | 50-55% | fBN264 | 6/30 = 20% |
| 4) | FA025-2-17 | 10% | 27% | fBN264 | |
| 5) | FA025-3-5 | 22-27% | 41-48% | fBN264 | |
| 6) | FA025-3-9 | 6-35% | 22-53% | fBN264 | |
| 7) | FA029-2-4 | 17-34% | 32-50% | pBN427 | |
| 8) | FA029-2-5 | 18-25% | 35-42% | pBN427 | |
| 9) | FA029-2-7 | 29% | 46% | pBN427 | |
| 10) | FA029-3-2 | 9-33% | 25-50% | pBN427 | 5/25 = 20% |
| 11) | FA029-3-4 | 26-29% | 40-43% | pBN427 | |

^{a}Typically, 20 kernels from 4 sibling cobs of each event were analyzed on the single kernel basis. The range indicates the lowest to the highest stearate content from the single kernel result of that event.
^{b}Total saturate fatty acids = 16:0 + 18:0 + 20:0.
^{c}f = purified fragment, p = intact plasmid DNA.
^{d}Co-suppression frequency = total number of events showing positive phenotype/total number of basta resistant clones generated from the respective transformation experiment.

Transgenic phenotypes in the new events were determined by the lipid composition in single kernels harvested from fully matured cobs using the same GC method described above. The sampling was non-destructive because only very small pieces of embryos were cut out from individual kernels and used for fatty acid composition assays. The kernels remain viable and can be planted in either the greenhouse or the field for propogatiom of the next generation.

Table 4 shows transgenic events identified with high stearate (and high total saturate fatty acids) phenotypes at the R0:1 generation. Typically, lipid assays were performed on 5-20 kernels from each cob, taken from 4-6 cobs from sibling plants for each transgenic event. The stearate and total saturate fatty acid contents are shown as percentage in oil, and the ranges presented indicate the lowest to highest percentages among all the single kernels analyzed in the event.

The results indicate that a consistently high frequency (10-20%) of co-suppression events may be obtained in corn (Table 4 and 6), whether using intact plasmid DNA or purified fragment. However, a small portion of vector DNA contamination may still be present in the preparations of purified fragment, and Southern blot analysis may be performed to verify the events truly free of a bacterial selectable marker. The Southern blot analysis that were performed indicated that use of a DNA fragment tends to generate events with simpler insertion patterns (one or few copies transgene insertion), than using the intact plasmid DNA. The latter may form complex concatemers and integrate together into the plant genome when used in the biolistic method, resulting in a complex insertion locus which may cause some transgene instability.

c. Transgenic Corn with a High Oleic Acid Content in Grains

Corn callus was co-bombarded with pBN257 DNA (SEQ ID NO:58) and a bar gene fragment, transgenic corn plants were produced, and R0:1 kernels were harvested and lipid composition analyzed as described above.

One transgenic event, FA014-5-1, was identified with a high oleate phenotype. FIG. 6 shows a typical example of segregation of R0:1 seeds harvested from a single cob, and their corresponding phenotypes. The cob was harvested from a wild type female plant (LH132), pollinated with pollen from a transgenic plant of line FA014-5-1. The ratio of positive phenotype: wild-type=1:1, indicating that line FA014-5-1 contains a single locus insertion, and the high oleate transgene trait may be dominant. The lipid profile of the positive phenotype is, on average, 12% 16:0, 1.3% 18 0, 70% 18:1, 15% 18:2, and 1.4% 18:3. The highest content of oleic acid found in samples taken from this cob was 81%, and in one of other cobs the content of oleic acid in some of the kernels was 83%. Accumulation of high levels of oleic acid is at the expense of linoleate, as shown in FIG. 6. There is about 2-4% decrease in palmitic acid, without any major change in 18:0, 18:3, 20:0 or 20:1 contents.

R3:4 kernels were harvested from homozygous plants, with the lipid composition as 10% 16:0, 1.5% 18:0,68% 18:1, 19% 18:2, and0.8% 18:3. The compositionresult is similar to that of the heterozygous R0:1 with a 2% lower oleate content, indicating that genotypic background may influence the transgenic phenotype. When the transgenic homozygous R3 plants were used as the pollen source, and crossed onto the high oil inbred lines QX47 (which possesses a total oil content of 14%), QHlO2 (which possesses a total oil content of 9%), or a hybrid line 34K77 in the TopCross® grain production method (U.S. Pat. Nos. 5,704,160 and 5,706,603), the respective lipid composition of kernels in each crossing are shown in Table 5. Oleate content in kernels from pure QX47 line is ˜43%, and the crossing of FA014-5-1 with this line also resulted in a higher oleate content in the grains (79% versus 68% from kernels of the homozygous FA014-5-1 plants). The total oil content of grains from crossing FA014-5-1 to QX47 is 8%-10%, and is 6%-7% from crossing FA014-5-1 to QH102.

TABLE 5
Kernel lipid composition in RO:1, homozygous R3:4,
and various crossing of FA014-5-1.

| | Phenotype | | | | |
|---|---|---|---|---|---|
| Genotype | 16:0 | 18:0 | 18:1 | 18:2 | 18:3 |
| R0:1 × LH132 | 12 | 1.3 | 70 | 15 | 1.7 |
| R3:4 selfed^{a} | 10 | 1.5 | 68 | 19 | 0.8 |
| QX47(HO) × R3 | 9 | 2 | 79 | 10 | 0.4 |
| QH102(HO) × R3 | 10 | 2 | 71 | 16 | 0.5 |
| 34K77 (TC) × R3 | 10 | 1 | 71 | 16 | 0.7 |
| WT^{b} | 15 | 1.2 | 18 | 65 | 0.7 |

^{a}The kernels were from selfed homozygous R3 plants. The same homozygous plants were used as the pollen source for the crossing with the female plants listed below.
^{b}A few 34K77 hybrid plants were selfed to obtain the wild-type kernels as the control.

Using similar processes, new transgenic events with high oleate phenotypes were generated (Table 6). The trait gene constructs used in these experiments are from either pBN257 or pBN428 (FIG. 7B). The transgene sequence in Sal I fragment (position 44-3468) of pBN428 is identical to the Sal I fragment of pBN257 (position 2-3426), except that pBN428 is using a vector backbone with a hygromycin resistance selectable marker gene (HPT, from pKS17, described in WO94/11516), versus the ampicillin selection in pBN257. The transgene prepared for bombardment was either the restriction enzyme digested and agarose gel purified DNA fragment, or the intact plasmid DNA as indicated in Table 6. The restriction enzyme used to cut out the transgene may be Sal I or Xba I, which release a transcriptionally functional transgene fragment of 3.4 kb, and can be purified by agarose gel electrophoresis.

TABLE 6
Transgenic events with high oleate phenotype

| | Transgenic events | Oleate^{a} | Construct^{b} | Co-suppression freq.^{c} |
|---|---|---|---|---|
| | Wild-type | ˜22% | | |
| 1) | FA014-5-1 | ˜70% | pBN257 | 1/10 = 10% |
| 2) | FA027-1-9 | 60-69% | fBN257 | |
| 3) | FA027-4-1 | 79-87% | fBN257 | 3/20 = 15% |
| 4) | FA027-4-5 | 81-87% | fBN257 | |
| 5) | FA028-1-8 | 39-63% | pBN428 | |
| 6) | FA028-1-10 | 50-55% | pBN428 | |
| 7) | FA028-3-1 | 64-78% | pBN428 | 4/32 = 13% |
| 8) | FA028-3-3 | 30-83% | pBN428 | |
| 9) | FA030-2-1 | 78-82% | fBN428 | |
| 10) | FA030-2-9 | 82-83% | fBN428 | 6/61 = 10% |
| 11) | FA030-3-1 | 80-84% | fBN428 | |
| 12) | FA030-3-3 | 40-68% | fBN428 | |
| 13) | FA030-4-25 | 42-77% | fBN428 | |
| 14) | FA030-5-17 | 71-86% | fBN428 | |
| 15) | FA031-5-8 | 58-76% | fBN428 | 1/6 = 17% |

^{a}Typically, 20 kernels from 4 sibling cobs of each event were analyzed on the single kernel basis. The range indicates the lowest to the highest stearate content from the single kernel result of that event.
^{b}f = purified fragment, p = intact plasmid DNA.
^{c}Co-suppression frequency = total number of events showing positive phenotype/total number of basta resistant clones generated from the respective transformation experiment.

Two of the high oleate events, FA027-4-1 and FA027-4-5 were carried forward to the R1:2 generation. The oleate content of kernels from these progenies indicated a consistent high oleate phenotype (81-87% oleate by single kernel analyses).

d. Transgenic Corn with High Levels of Saturated and Oleic Acids in Kernels

Corn with a high level of saturated fatty acid and a high level of oleic acid in kernels may be produced by crossing a high saturate transgenic line (FA013-2-4 or FA013-3-2) and the high oleate transgenic line (FA014-5-1), or by crossing the high saturate transgenic line with a high oleic acid mutant such a lines B730L or AEC2720L (WO95/22598).

An alternative approach for obtaining a corn plant high in both saturated fatty acids and oleic acid is to create a transgenic line with a transgene construct containing the filsed fad2 and delta-9 desaturase genes, such as in pBN412 or pBN414 or pBN431 (FIG. 7C), or the transformation may be done by co-bombardment with both pBN257 (or pBN428) and pBN264 (or pBN427 or pBN262).

Transgenic events comprisng the chimeric gene from pBN431 possess a phenotype in which the total saturate level is not less than about 30% of the total seed oil content, the stearic acid level is in the range from about 11% to 31% of the total seed oil content and the oleic acid level is in the range from about 27% to about 37% of the total seed oil content. It is believed that oils may be obtained which possess an oleic acid level in the range from about 35% to about 45% of the total seed oil content by crossing these transgenic events with a line having a high oleic acid phenotype, e.g., any of the transgenic events set forth in Table 6 above, or B730L or AEC2720L which are referred to above.

The high stearic acid and high oleic acid corn oil resulting from such a transgenic event may be used in a blended or unblended form as a margarine or shortening, and it may be blended with a high palmitic acid fat to form a cocoa butter substitute.

What is claimed is:

1. An isolated nucleic acid fragment comprising a corn oleosin promoter wherein said promoter can be full length or partial and further wherein said promoter comprises a nucleotide sequence corresponding substantially to the nucleotide sequence in any of SEQ ID NOS: 19 or 38-49 or said promoter comprises a fragment or subfragment that is substantially similar and functionally equivalent to any of the nucleotide sequences set forth in SEQ ID NOS:19 or 38-49.

2. The fragment or subfragment of claim 1 wherein said fragment or subfragment hybridizes to the nucleotide sequence set forth in SEQ ID NOS:19 or 38-49 under moderately stringent conditions.

3. An isolated nucleic acid fragment encoding a corn delta-9 stearoyl-ACP desaturase corresponding substantially to a nucleotide sequence set forth in any of SEQ ID NOS:8 and 10 or any functionally equivalent subfragment thereof.

4. An isolated nucleic acid fragment encoding a corn delta-12 desaturase corresponding substantially to the nucleotide sequence set forth in SEQ ID NO:2 or any functionally equivalent subfragment thereof.

5. A chimeric gene comprising the nucleic acid fragment of claim 3 or the reverse complement of the nucleic acid fragment of claim 3 operably linked to suitable regulatory sequences wherein expression of the chimeric gene results in an altered corn stearic acid phenotype.

6. A chimeric gene comprising the nucleic acid fragment of claim 4 or the reverse complement of the nucleic acid fragment of claim 4 operably linked to suitable regulatory sequences wherein expression of the chimeric gene results in an altered corn oleic acid phenotype.

7. The chimeric gene of claim 5 further comprising the nucleic acid fragment of claim 1 or 2.

8. The chimeric gene of claim 6 further comprising the nucleic acid fragment of claim 1 or 2.

9. The chimeric gene of claim 5 further comprising a shrunken 1 intron/exon.

10. The chimeric gene of claim 6 further comprising a shrunken 1 intron/exon.

11. The chimeric gene of claim 7 further comprising a shrunken 1 intron/exon.

12. The chimeric gene of claim 8 further comprising a shrunken 1 intron/exon.

13. A chimeric gene comprising the nucleic acid fragment of claim 3 or the reverse complement thereof and a nucleic acid fragment encoding a corn delta-12 desaturase, any functionally equivalent subfragment thereof or the reverse complement of said fragment or subfragment wherein said fragments are operably linked and further wherein expression of the chimeric gene results in an altered corn oil phenotype.

14. The chimeric gene of claim 13 wherein the nucleic acid fragment encoding a corn delta-12 desaturase corresponds substantially to the nucleotide sequence set forth in SEQ ID NO:2 or any functionally equivalent subfragment thereof.

15. A chimeric gene comprising the nucleic acid fragment of claim 1 or 2, the nucleic acid fragment of claim 3 or the reverse complement thereof and a nucleic acid sequence encoding a corn delta-12 desaturase, any functionally equivalent subfragment thereof or the reverse complement of said fragment or subfragment wherein said fragments are operably linked and further wherein expression of the chimeric gene results in an altered corn oil phenotype.

16. The chimeric gene of claim 15 wherein the nucleic acid fragment encoding a corn delta-12 desaturase enzyme corresponds substantially to the nucleotide sequence set forth in SEQ ID NO:2 or any functionally equivalent subfragment thereof.

17. A chimeric gene comprising the nucleic acid fragment of claim 3, a nucleic acid sequence encoding a corn delta-12 desaturase, any functionally equivalent subfragment thereof or the reverse complement of said fragment or subfragment and a shrunken 1 intron/exon wherein said fragments are operably linked and further wherein expression of the chimeric gene results in an altered corn oil phenotype.

18. The chimeric gene of claim 17 wherein the nucleic acid fragment encoding the delta-12 desaturase corresponds substantially to the nucleotide sequence set forth in SEQ ID NO:2 or any functionally equivalent subfragment thereof.

19. A chimeric gene comprising the nucleic acid fragment of claim 1 or 2, the nucleic acid fragment of claim 3 or the reverse complement thereof, a nucleic acid sequence encoding a corn delta-12 desaturase, any functionally equivalent subfragment thereof, or the reverse complement of said fragment or subfragment and a shrunken 1 intron/exon wherein said fragments are operably linked and further wherein expression of the chimeric gene results in an altered corn oil phenotype.

20. The chimeric gene of claim 19 wherein the nucleic acid fragment encoding the delta-12 desaturase corresponds substantially to the nucleotide sequence set forth in SEQ ID NO:2 or any functionally equivalent subfragment thereof.

21. A chimeric gene comprising the nucleic acid fragment of claim 1 or 2, the nucleic acid fragment of claim 3 or the reverse complement thereof, the nucleic acid fragment of claim 4 or the complement thereof and a shrunken 1 intron/exon wherein said fragments are operably linked and further wherein the expression of the chimeric gene results in an altered corn oil phenotype.

22. A chimeric gene comprising an isolated nucleic acid fragment encoding a corn delta-12 desaturase corresponding substantially to the nucleotide sequence set forth in SEQ ID NO: 1, a functionally equivalent subfragment thereof or the reverse complement of said fragment or subfragment, or an isolated nucleic acid fragment corresponding substantially to the nucleotide sequence set forth in SEQ ID NO:58 or 59 or a functionally equivalent subfragment thereof or the reverse complement of such fragment or subfragment, the nucleic acid fragment of claim 1 or 2 and shrunken 1 intron/exon wherein said fragments are operably linked and further wherein expression of the chimeric gene results in an altered corn oleic acid phenotype.

23. A corn plant or plant parts thereof comprising the chimeric gene of claim 5.

24. A corn grain obtained from the plant of claim 23 wherein said grain has either a stearic acid content of not less than about 20% of the total oil content or a total saturate content of not less than about 35% of the total oil content.

25. A corn plant or plant parts thereof comprising the chimeric gene of claim 6.

26. A corn grain obtained from the plant of claim 25 wherein said grain has an oleic acid content of not less than about 60% of the total oil content.

27. A corn plant or plant parts thereof comprising the chimeric gene of claim 7.

28. A corn grain obtained from the plant of claim 27 wherein said grain has either a stearic acid content of not less than about 20% of the total oil content or a total saturate content of not less than about 35% of the total oil content.

29. A corn plant or plant parts thereof comprising the chimeric gene of claim 8.

30. A corn grain obtained from the plant of claim 29 wherein said grain has an oleic acid content of not less than about 60% of the total oil content.

31. A corn plant or plant parts thereof comprising the chimeric gene of claim 9.

32. A corn grain obtained from the plant of claim 31 wherein said grain has either a stearic acid content of not less than about 20% of the total oil content or a total saturate content of not less than about 35% of the total oil content.

33. A corn plant or plant parts thereof comprising the chimeric gene of claim 10.

34. A corn grain obtained from the plant of claim 33 wherein said grain has an oleic acid content of not less than about 60% of the total oil content.

35. A corn plant or plant parts thereof comprising the chimeric gene of claim 11.

36. A corn grain obtained from the plant of claim 35-wherein said grain has either a stearic acid content of not less than about 20% of the total oil content or a total saturate content of not less than about 35% of the total oil content.

37. A corn plant or plant parts thereof comprising the chimeric gene of claim 12.

38. A corn grain obtained from the plant of claim 37 wherein said grain has an oleic acid content of not less than about 60% of the total oil content.

39. A corn plant or plant parts thereof comprising the chimeric gene of claim 13.

40. A corn grain obtained from the plant of claim 39 wherein said grain has a total saturate content of not less than about 30% of the total oil content and an oleic acid content of not less than about 30% of the total oil content.

41. A corn plant or plant parts thereof comprising the chimeric gene of claim 14.

42. A corn grain obtained from the plant of claim 41 wherein said grain has a total saturate content of not less than about 30% of the total oil content and an oleic acid content of not less than about 30% of the total oil content.

43. A corn plant or plant parts thereof comprising the chimeric gene of claim 15.

44. A corn grain obtained from the plant of claim 43 wherein said grain has a total saturate content of not less than about 30% of the total oil content and an oleic acid content of not less than about 30% of the total oil content.

45. A corn plant or plant parts thereof comprising the chimeric gene of claim 16.

46. A corn grain obtained from the plant of claim 45 wherein said grain has a total saturate content of not less than about 30% of the total oil content and an oleic acid content of not less than about 30% of the total oil content.

47. A corn plant or plant parts thereof comprising the chimeric gene of claim 17.

48. A corn grain obtained from the plant of claim 47 wherein said grain has a total saturate content of not less than about 30% of the total oil content and an oleic acid content of not less than about 30% of the total oil content.

49. A corn plant or plant parts thereof comprising the chimeric gene of claim 18.

50. A corn grain obtained from the plant of claim 49 wherein said grain has a total saturate content of not less than about 30% of the total oil content and an oleic acid content of not less than about 30% of the total oil content.

51. A corn plant or plant parts thereof comprising the chimeric gene of claim 19.

52. A corn grain obtained from the plant of claim 51 wherein said grain has a total saturate content of not less than about 30% of the total oil content and an oleic acid content of not less than about 30% of the total oil content.

53. A corn plant or plant parts thereof comprising the chimeric gene of claim 20.

54. A corn grain obtained from the plant of claim 53 wherein said grain has a total saturate content of not less than about 30% of the total oil content and an oleic acid content of not less than about 30% of the total oil content.

55. A corn plant or plant parts thereof comprising the chimeric gene of claim 21.

56. A corn grain obtained from the plant of claim 55 wherein said grain has a total saturate content of not less than about 30% of the total oil content and an oleic acid content of not less than about 30% of the total oil content.

57. A corn plant or plant parts thereof comprising one chimeric gene selected from the chimeric genes of claims 5, 7, 9, and 11 and one chimeric gene selected from the chimeric genes of claims 6, 8, 10, and 12.

58. A corn grain obtained from the plant of claim 57 wherein said grain has a total saturate content of not less than about 30% of the total oil content and an oleic acid content of not less than about 30% of the total oil content.

59. A corn plant or plant parts thereof comprising the chimeric gene of claim 22.

60. A corn grain obtained from the plant of claim 59 wherein said grain has an oleic acid content of not less than about 60% of the total oil content.

61. Seeds of the corn plant of claim 23 or 25.

62. Seeds of the corn plant of claim 27.

63. Seeds of the corn plant of claim 29

64. Seeds of the corn plant of claim 31.

65. Seeds of the corn plant of claim 33.

66. Seeds of the corn plant of claim 35.

67. Seeds of the corn plant of claim 37.

68. Seeds of the corn plant of claim 39.

69. Seeds of the corn plant of claim 41.

70. Seeds of the corn plant of claim 43.

71. Seeds of the corn plant of claim 45

72. Seeds of the corn plant of claim 47.

73. Seeds of the corn plant of claim 49.

74. Seeds of the corn plant of claim 51.

75. Seeds of the corn plant of claim 53.

76. Seeds of the corn plant of claim 55.

77. Seeds of the corn plant of claim 57.

78. Seeds of the corn plant of claim 59.

79. Oil obtained from the grain of the corn plants of claims 23 or 25.

80. Oil obtained from the grain of the corn plant of claim 27.

81. Oil obtained from the grain of the corn plant of claim 29.

82. Oil obtained from the grain of corn the plant of claim 31.

83. Oil obtained from the grain of the corn plant of claim 33.

84. Oil obtained from the grain of the corn plant of claim 35.

85. Oil obtained from the grain of the corn plant of claim 37.

86. Oil obtained from the grain of the corn plant of claim 39.

87. Oil obtained from the grain of the corn plant of claim 41.

88. Oil obtained from the grain of corn the plant of claim 43.

89. Oil obtained from the grain of the corn plant of claim 45.

90. Oil obtained from the grain of the corn plant of claim 47.

91. Oil obtained from the grain of corn the plant of claim 49.

92. Oil obtained from the grain of the corn plant of claim 51.

93. Oil obtained from the grain of the corn plant of claim 53.

94. Oil obtained from the grain of the corn plant of claim 55.

95. Oil obtained from the grain of the corn plant of claim 57.

96. Oil obtained from the grain of the corn plant of claim 59.

97. Animal feed derived from the processing of corn grain of claim 61.

98. Animal feed derived from the processing of corn grain of claim 62.

99. Animal feed derived from the processing of corn grain of claim 63.

100. Animal feed derived from the processing of corn grain of claim 64.

101. Animal feed derived from the processing of corn grain of claim 65.

102. Animal feed derived from the processing of corn grain of claim 66.

103. Animal feed derived from the processing of corn grain of claim 67.

104. Animal feed derived from the processing of corn grain of claim 68.

105. Animal feed derived from the processing of corn grain of claim 69.

106. Animal feed derived from the processing of corn grain of claim 70.

107. Animal feed derived from the processing of corn grain of claim 71.

108. Animal feed derived from the processing of corn grain of claim 72.

109. Animal feed derived from the processing of corn grain of claim 73.

110. Animal feed derived from the processing of corn grain of claim 74.

111. Animal feed derived from the processing of corn grain of claim 75.

112. Animal feed derived from the processing of corn grain of claim 76.

113. Animal feed derived from the processing of corn grain of claim 77.

114. Animal feed derived from the processing of corn grain of claim 78.

115. The use of the oil of claim 79 in food, animal feed, cooking or industrial applications.

116. The use of the oil of claim 80 in food, animal feed, cooking or industrial applications.

117. The use of the oil of claim 81 in food, animal feed, cooking or industrial applications.

118. The use of the oil of claim 82 in food, animal feed, cooking or industrial applications.

119. The use of the oil of claim 83 in food, animal feed, cooking or industrial applications.

120. The use of the oil of claim 84 in food, animal feed, cooking or industrial applications.

121. The use of the oil of claim 85 in food, animal feed, cooking or industrial applications.

122. The use of the oil of claim 86 in food, animal feed, cooking or industrial applications.

123. The use of the oil of claim 87 in food, animal feed, cooking or industrial applications.

124. The use of the oil of claim 88 in food, animal feed, cooking or industrial applications.

125. The use of the oil of claim 89 in food, animal feed, cooking or industrial applications.

126. The use of the oil of claim 90 in food, animal feed, cooking or industrial applications.

127. The use of the oil of claim 91 in food, animal feed, cooking or industrial applications.

128. The use of the oil of claim 92 in food, animal feed, cooking or industrial applications.

129. The use of the oil of claim 93 in food, animal feed, cooking or industrial applications.

130. The use of the oil of claim 94 in food, animal feed, cooking or industrial applications.

131. The use of the oil of claim 95 in food, animal feed, cooking or industrial applications.

132. The use of the oil of claim 96 in food, animal feed, cooking or industrial applications.

133. Products made from the hydrogenation, fractionation, interesterification, or hydrolysis of oil obtained from the grain of the plant of claim 23 or 25.

134. Products made from the hydrogenation, fractionation, interesterification, or hydrolysis of oil obtained from the grain of the plant of claim 27.

135. Products made from the hydrogenation, fractionation, interesterification, or hydrolysis of oil obtained from the grain of the plant of claim 29.

136. Products made from the hydrogenation, fractionation, interesterification, or hydrolysis of oil obtained from the grain of the plant of claim 31.

137. Products made from the hydrogenation, fractionation, interesterification, or hydrolysis of oil obtained from the grain of the plant of claim 33.

138. Products made from the hydrogenation, fractionation, interesterification, or hydrolysis of oil obtained from the grain of the plant of claim 35.

139. Products made from the hydrogenation, fractionation, interesterification, or hydrolysis of oil obtained from the grain of the plant of claim 37.

140. Products made from the hydrogenation, fractionation, interesterification, or hydrolysis of oil obtained from the grain of the plant of claim 39.

141. Products made from the hydrogenation, fractionation, interesterification, or hydrolysis of oil obtained from the grain of the plant of claim 41.

142. Products made from the hydrogenation, fractionation, interesterification, or hydrolysis of oil obtained from the grain of the plant of claim 43.

143. Products made from the hydrogenation, fractionation, interesterification, or hydrolysis of oil obtained from the grain of the plant of claim 45.

144. Products made from the hydrogenation, fractionation, interesterification, or hydrolysis of oil obtained from the grain of the plant of claim 47.

145. Products made from the hydrogenation, fractionation, interesterification, or hydrolysis of oil obtained from the grain of the plant of claim 49.

146. Products made from the hydrogenation, fractionation, interesterification, or hydrolysis of oil obtained from the grain of the plant of claim 51.

147. Products made from the hydrogenation, fractionation, interesterification, or hydrolysis of oil obtained from the grain of the plant of claim 53.

148. Products made from the hydrogenation, fractionation, interesterification, or hydrolysis of oil obtained from the grain of the plant of claim 55.

149. Products made from the hydrogenation, fractionation, interesterification, or hydrolysis of oil obtained from the grain of the plant of claim 57.

150. Products made from the hydrogenation, fractionation, interesterification, or hydrolysis of oil obtained from the grain of the plant of claim 59.

151. A method of improving the carcass quality of an animal by feeding the animal a carcass quality improving amount of the animal feed of claim 97.

152. A method of improving the carcass quality of an animal by feeding the animal a carcass quality improving amount of the animal feed of claim 98.

153. A method of improving the carcass quality of an animal by feeding the animal a carcass quality improving amount of the animal feed of claim 99.

154. A method of improving the carcass quality of an animal by feeding the animal a carcass quality improving amount of the animal feed of claim 100.

155. A method of improving the carcass quality of an animal by feeding the animal a carcass quality improving amount of the animal feed of claim 101.

156. A method of improving the carcass quality of an animal by feeding the animal a carcass quality improving amount of the animal feed of claim 102.

157. A method of improving the carcass quality of an animal by feeding the animal a carcass quality improving amount of the animal feed of claim 103.

158. A method of improving the carcass quality of an animal by feeding the animal a carcass quality improving amount of the animal feed of claim 104.

159. A method of improving the carcass quality of an animal by feeding the animal a carcass quality improving amount of the animal feed of claim 105.

160. A method of improving the carcass quality of an animal by feeding the animal a carcass quality improving amount of the animal feed of claim 106.

161. A method of improving the carcass quality of an animal by feeding the animal a carcass quality improving amount of the animal feed of claim 107.

162. A method of improving the carcass quality of an animal by feeding the animal a carcass quality improving amount of the animal feed of claim 108.

163. A method of improving the carcass quality of an animal by feeding the animal a carcass quality improving amount of the animal feed of claim 109.

164. A method of improving the carcass quality of an animal by feeding the animal a carcass quality improving amount of the animal feed of claim 110.

165. A method of improving the carcass quality of an animal by feeding the animal a carcass quality improving amount of the animal feed of claim 111.

166. A method of improving the carcass quality of an animal by feeding the animal a carcass quality improving amount of the animal feed of claim 112.

167. A method of improving the carcass quality of an animal by feeding the animal a carcass quality improving amount of the animal feed of claim 113.

168. A method of improving the carcass quality of an animal by feeding the animal a carcass quality improving amount of the animal feed of claim 114.

169. A corn grain comprising in its genome the chimeric gene of claim 22 wherein said corn grain has an oil content in the range from about 6% to about 10% on a dry matter basis and further wherein said oil is comprised of not less than about 60% oleic acid of the total oil content of the seed.

170. Animal feed derived from the processing of the corn grain of claim 169.

171. A method for improving the carcass quality of an animal by feeding the animal a carcass quality improving amount of the animal feed of claim 170.


SEQUENCE LISTING (59 sequences)

SEQ ID NO: 1 - DNA, 1790 bp, Zea mays
cggcctctcccctccctcctccctgcaaatcctgcagacaccaccgctcgtttttctctccgggacaggagaaaaggggagagagaggtgaggcgcggtgtccgcccgatctgctctgccccgacgcagctgttacgacctcctcagtctcagtcaggagcaagatgggtgccggcggcaggatgaccgagaaggagcgggagaagcaggagcagctcgcccgagctaccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactactcgctcctggacgacgtggtcggcctggtgctgcactcgtcgctcatggtgccctacttctcgtggaagtacagccaccggcgccaccactccaacacggggtccctggagcgcgacgaggtgttcgtgcccaagaagaaggaggcgctgccgtggtacaccccgtacgtgtacaacaacccggtcggccgggtggtgcacatcgtggtgcagctcaccctcgggtggccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtacaagctggcggcggcgttcggggtctggtgggtggtgcgcgtgtacgccgtgccgctgctgatcgtgaacgcgtggctggtgctcatcacctacctgcagcacacccacccgtcgctcccccactacgactcgagcgagtgggactggctgcgcggcgcgctggccaccatggaccgcgactacggcatcctcaaccgcgtgttccacaacatcacggacacgcacgtcgcgcaccacctcttctccaccatgccgcactaccacgccatggaggccaccaaggcgatcaggcccatcctcggcgactactaccacttcgacccgacccctgtcgccaaggcgacctggcgcgaggccggggaatgcatctacgtcgagcccgaggaccgcaagggcgtcttctggtacaacaagaagttctagccgccgccgctcgcagagctgaggacgctaccgtaggaatgggagcagaaaccaggaggaggagacggtactcgccccaaagtctccgtcaacctatctaatcgttagtcgtcagtcttttagacgggaagagagatcatttgggcacagagacgaaggcttactgcagtgccatcgctagagctgccatcaagtacaagtaggcaaattcgtcaacttagtgtgtcccatgttgtttttcttagtcgtccgctgctgtaggctttccggcggcggtcgtttgtgtggttggcatccgtggccatgcctgtgcgtgcgtggccgcgcttgtcgtgtgcgtctgtcgtcgcgttggcgtcgtctcttcgtgctccccgtgtgttgttgtaaaacaagaagatgttttctggtgtctttggcggaataacagatcgtccgaacgaaaaaaaaaaaaaaaaaaaa

SEQ ID NO: 2 - DNA, 1733 bp, Zea mays
  CDS (176)..(1351)
tcctccctcctcctccctgcaaatcgccaaatcctcaggcaccaccgctcgttttcctgtgcggggaacaggagagaaggggagagaccgagagagggggaggcgcggcgtccgccggatctgctccgacccccgacgcagcctgtcacgccgtcctcactctcagccagcgaaaatgggtgccggcggcaggatgaccgagaaggagcgggaggagcaggagcaggagcaggtcgcccgtgctaccggcggtggcgcggcagtgcagcggtcgccggtggagaagccgccgttcacgttggggcagatcaagaaggcgatcccgccgcactgcttcgagcgctccgtgctgaggtccttctcgtacgtggcccacgacctggcgctcgccgcggcgctcctctacctcgcggtggccgtgataccggcgctaccctgcccgctccgctacgcggcctggccgctgtactgggtggcccaggggtgcgtgtgcacgggcgtgtgggtgatcgcgcacgagtgcggccaccacgccttctccgaccacgcgctcctggacgacgccgtcggcctggcgctgcactcggcgctgctggtgccctacttctcgtggaagtacagccaccggcgccaccactccaacacggggtccctggagcgcgacgaggtgttcgtgccgaggaccaaggaggcgctgccgtggtacgccccgtacgtgcacggcagccccgcgggccggctggcgcacgtcgccgtgcagctcaccctgggctggccgctgtacctggccaccaacgcgtcggggcgcccgtacccgcgcttcgcctgccacttcgacccctacggcccgatctacggcgaccgggagcgcgcccagatcttcgtctcggacgccggcgtcgcggccgtggcgttcgggctgtacaagctggcggcggcgttcgggctctggtgggtggtgcgcgtgtacgccgtgccgctgctgatcgtcaacgcgtggctggtgctcatcacgtacctgcagcacacccacccggcgctgccccactacgactcgggcgagtgggactggctgcgcggcgcgctcgccaccgtcgaccgcgactacggcgtcctcaaccgcgtgttccaccacatcacggacacgcacgtcgcgcaccacctcttctccaccatgccgcactaccacgccgtggaggccaccagggcgatcaggcccgtcctcggcgagtactaccagttcgacccgacccctgtcgccaaggccacctggcgcgaggccagggagtgcatctacgtcgagcctgagaaccgcaaccgcaagggcgtcttctggtacaacagcaagttctagccgccgcttgctttttccctaggaatgggaggagaaatcaggatgagaagatggtcctgtctccatctacctgtctaatggttagtcaccagtctttagacaggaagagagcatttgggcttcagaaaaggaggcttactgcactactgcagtgccatcgctagatcctaggcaaattcagtgtgctcctgtgccccatggctgtgagctttgggtactctcaagtagtcaagttctcttgtttttgtttttagtcgtccgctgttgtaggcttgccggcggcggtcgttcgcgtggccgcgccttgtcgtgtgcgtctctcgccactctcttcgtgctccccaaaaaaaaaaaaaaaaaaaaaaaaaaaaaaaaaaaa

SEQ ID NO: 3 - protein, 392 aa, Zea mays
MGAGGRMTEKEREEQEQEQVARATGGGAAVQRSPVEKPPFTLGQIKKAIPPHCFERSVLRSFSYVAHDLALAAALLYLAVAVIPALPCPLRYAAWPLYWVAQGCVCTGVWVIAHECGHHAFSDHALLDDAVGLALHSALLVPYFSWKYSHRRHHSNTGSLERDEVFVPRTKEALPWYAPYVHGSPAGRLAHVAVQLTLGWPLYLATNASGRPYPRFACHFDPYGPIYGDRERAQIFVSDAGVAAVAFGLYKLAAAFGLWWVVRVYAVPLLIVNAWLVLITYLQHTHPALPHYDSGEWDWLRGALATVDRDYGVLNRVFHHITDTHVAHHLFSTMPHYHAVEATRAIRPVLGEYYQFDPTPVAKATWREARECIYVEPENRNRKGVFWYNSKF

SEQ ID NO: 4 - DNA, 12313 bp, Zea mays
ttgtgatgttgtcaggggggcggagctatggaaaaacgccagcaacgcggctttttacggttcctggcttttgctggcttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccccttgcagcagagagcaagttccaacaatacccccaaccacccaccattcattgcatccaagttttctaacttcccacaacttacaagagctatagcattcaatacaagacacaccaaagagatcaaatcctctcccaagtccatagatcatttccaatcaaataatgactagtgagagggtgacttgtgttcatttgagctcttgcgcttggattgcttctttttctcattctttcttgtgatcaactcaattgtaaccgagacaagagacaccaattgtgtggtggtccttgcggggactttgtgtctcgtttgattgagaagagaagctcactcggtctaagtgatcgtttgagagagggaaagggttgaaagagacccggtctttgtgaccacctcaacgggggagtaggtttgcaagaaccgaacctcggtaaaacaaatattttgcttacaatttgtttttcgccctctctctcggactcgttaatatttctaacgctaacccggcttgtagttgtgcttaagtttataaatttcagattcgccctattcacccccctctaggcgactttcagtaccgttatatatgctttcgatttatcctgcccctaagtcagttactagaaagattgatattcttaggaggcgtcttctttggcaagggggtcgtcagtccaaaaaaattcatttagttgattggttgtcggtgtgctctcccaaaaagtcagggaggtctgggtgttctgaatctcgattttatgaatgattccttaatgactaaatggctttggaatattgaaacttcgaatggcttatggcaaaaaattattaccagtaaatatattaagggaaaaccccttattttgatcaagcaaagacaaggtgattcacacttctggaaaaaaaattctgagtctgcgtgataatttttacaaattttgcaaatctggggtgggaaacggtttgaagactagcttttggaagagtatctggattggaaatctgcccctgtctgttcagtttcctgttctatttgacttgtcttatgacaaagacattacggttaatgatgtcatggcttctaattttgaggttcttacatttagaagaaggattgttggtaatctgagggttctaatggatgagttggtgagttgttgcaatcatgtgttcttgtctgatcaggaggacagaattgtgtggagtctggggagaaaaggcttttctattaattttatttaaaaaagaaaatggcagatcaagttttgatttcatataagttcttgtggaaaatcaagattttcatgtggttggttgtgagaaataaaattcttactaaagacaatatgaagaaaaggaactggaatggttctttggaatgttgtttctgtggcgtggatgaatccattgatcatttattctttcattgtcccattgcgagatatatgtggagagtgattcaagtggccttgaatctgaggatgattccaagtagtattagcaacctttatgacaaccggttatgtagaccaaaagataatattgctaatctggttttgtttggctgtggagctatgttctgggcaatttggcgcactagaaatgattggtgctttgggaataaaactatgcttgatccctctaacatcatttttctttgctgcttctggctggattcctaggctattcgacagagaaagaaggagcaaaaaatagtggtccaaggaagcaagctaatctgaaagacaacaagtgaagcattcagccgagcgtttgggtggtgcccgatagacaggcgtatttctggttgatcttaagctggaacttgaatgatggtgctggtgctatcctttcttttggtggttgtcttggttcagtatcttttgttgcaccagtctgtcatgatatgattgtaaataagaaaggcttatgttgttaatcgtaagtcaaactttattcgctatcataggtcctccactgatctagtttgatagtgttaggagtctagatagagatctgaccttgttcgatttttttggtttattggtcgcatgagtactgttgtttcaaactttcatatttcttaatgaaataggggcttcgcccctacaactctgatcactttcacttgcatacgggagacctctccaattcatactgtgtgttgggggggggggtgggggggacaagaataacgagagaaaaaaatctgagctttaccattacagaggtcagaggttacgaacagctgcatccaccgtcaaaatgcgccagtgcacccacgtcctgttggattaatgtgggcttggcccaaattaatattcaataatagtcaatgctaatggcccactttaatgctatggtgtactaattatttagtaccatattggaagttcaaaggacaaatcaatcaacttaaataggtggaccattggtgcatctattgagaagttgagaaaagaatgaaagactgccacacgcgcgcgcgcgccgccgccgccggccgggcccgtggccgtggccgtggccgtggctcgtggctcgtggtagatcggaccttggtccgaatattcctttcaaacggttgtgcattttgcctggattgatgaccgtcataataaccgtctgtttcctgtcttatggctagtaacggacgtcagttactgtcgtcagtttccagttctaatgcgcgaccgtttctgtccgttgctcttctcccttcttctgaccggctataagaatggagagggagagctcttccagtcaggcgaatttatctcacgcgaattgcaaacaacacattccccgtcccatcttctgcgagcacagagagagtgggagagcaggcctccgaaatcaccgaccgcagagatacacttgcacgggtgtgcgggcgatcagatttttggggagcgtcttcgcgactgctcgcgtgatcgtccacagcttgctgttcgtcgcctacccaagttgacgcgtgctgctgttcttcttcccggcgaccgttcgagggactgcactgcgtacaccttcctgcaccgacttcgtacgactacatcgaacaaacacacgagatgtctcgtgtgaatggagccactggtgccttgagcatcggtccctccgctgggtacactctgttcttcgtatttgtgcatgtttcattgctgtttactgcttatgcgagtagttatacacacatgcacatacatgtcatcacatatatcacactgattttctggattaaattaaaactaaaaatgcctaactttctaacacgtccgagcatcaccgcttgcttgcgccctcggcggtctggaatctgcatgtcgccgggcgcggggcgccggcgcaccgcccccgccgtggtctgctacccgtcagtccgcgccacacttcttgaggagaacatcgtcgcacgcgggcacgcggcgtggccggcggtgacaactgcagagcatggtcgccacttgtcagttctgtcagcaagggtgccggtgccagtgccagcaccgagctcgctttgtttgcctgctgccagtgtggcagacatcggacgacggagctgtaggcgccatgcgcatactagatgggtatctttcggtgcttggaacttggttcacaggtggatgtctgcatgcacatcgtctctacagtctacactgaatcaagcacaccattacaccaatgcatttttctgttgcctgtatggagatagctgattagttcaccgaatgaagcacaccaacgtgcgtacttttccaaccagttgcgcttggagatagctgctggttagttcaccgaatccgcggcctaactccggacacatttttttcttctggtagatcgcatcacatgcttgctccccatcacgggctgcaaggtgccacccctcgctgcctgttccaggccatcaacaccgtgggtttggcaaccggtgttgcgctacccaatgcctgagaaaaatcgtggtacggcccaaccatggaagatcagccaaaatgagctcacatgaaactgcccaaaacaggaagagggtagttgaaataaaatgggttcagtgacggtacgaagtcagatttgaagaagtgcccaacgataatacatagttcaactacattcgtattatttttggacaaatcttcaggtcccaaattatttagttcaccgctgcaaactactatatggaaagatacgacgatcaatcaaaaggcaattttctttggtgaaccaatcgtttcacaagggaaatcaactacgccgatgtctgctgttttccttagggcctgttcgcttcttcaggaatgaacttggattcattcgagctcatcaaaatttatataaattagaaaagtaatccggctaagaactattccagggctccaatccgtgaaaaccgaacagagccttagagagcccgtctgttggataggagtatatagctttttgtttaagcttttttttcaatttctgatcaccagaagatgtcgcaaaactgttaaacatctaactttttaacctgtttctataagaatcattttagtcaaaattatctaaaatcaatatgaggacagaatcaaccgagtccttatgaaaaccgtcattttctatatcctaaatcatataaactattttatctttcttcacactttatctacatgaaactgtattccctacaaccatatttttctggcagtcagattctaaaaaaaatcctcacaaaaaagttgaaccaaactcgcgagccacgggcccgcgtccggcgctgcacgagctgtgtcacgcctcccggcctcccggggtccagccaaatagggctctacatgtgcacagggccagatttcacgtccgccgacgtggttacggcgtcacatgatcacatctggctcctccgggcccaggcgccagtgacgccgtgcccgcctctaaatagcgcctctctcccgggctgccggcggaaccgaggcggtcaggctccctcctccttcctcctccctgcaaatcctgcaggcaccaccgctcgttttcctgtccgggggacaggagagaaggggagagaccgagagagggtgaggcgcggcgtccgcccgatctgctccgccccccgaagcagcctgtcacgtcgtcctcactctcaggtacccgcatttagccttcctggattgttatggatcactagtgccccccctgccactgttccatagattgttccgaatggattggtgaggaatcgaccggcgttcggttctgggttgctgagcccggcaacgggctcgtggccggccgtcgattcggcagcggcactcgccgtcgcgccgcccggtcgggtcgggtcgggtctctgcaaactcgccgtagcgcctgccggtcgagctttgacaccgacctcaccggcgggcatccgcggccctgccgatgtggatttcaggttttgccccgatgaatccacgcttgttcctcaccagatctgtaggtatgattcagcgagtggtgccattcagatattttgcccgtgcaatgggaccgtgattgatctccgcacctcctgccgtgaccactcgttttgggaacatggcatgccacctttagccacgcccacgagctgacgagctcttcgcagctcccgtataaaaagctgcaacctttgcaggtctttgactccaaaggcggcctctttgtttcggcgctcgcccccctccatgttgggcatgatgcgttgcacttggtgcccgactcctctgttttctagctcctaattttttttgctgatgctactatagtactattagctaagcgcggagttggcgatgactgcgctcaagaatcgacctttggctcgggcaatcgatgcatggacgaagccacttgttttttttttctttggtcatgtttttgacatgcgaaactgcgaaggtggcagagtaggtggatctttctgtctatgttttggccctacttgagaggaagagacagtcgccaccgtgcaaagtcccaaaggcatccgacggtggcgcgtcgatcgttacgatgccagacaagcaccacggaacgagccacctcccccgcggccagcccgccatcgagcaggtcacgactgagattgaaagaaacgcgatctaatttttgtcttttcttttcctgtcccaagttccttaatacttgatacgtgagctctataacactagaggttttccattcggaaaaatatgttcgctaaagtcggtctctgattaaagtcggctgcttgacggctgcaactgtaatttatgtaatttattaaaacaaaaacactgtgttgacacctttttggaggcgccaatcacttcaaaagaaccggcggcggtgctctctggtcaggcgcggacggtccgcggcacagggccggacggtccgcgacctggcgtgaggcggcggtgctctctggtcaggcgcggacggtccgcggcacagggccggacggtccgcgacctggagcaggagctcgggttccctgcctgacggtcggacggtccgcgcgtgcgcaggggcggcggaagatcgccggcggcgcctggatctcgctcccgggagggaccccgtcggggaggagagatcctaggagttgtctaggctcgggccggccgacctagactcctctaatcgacgtagagtcgaggagaggcggagaatttggggattggaatactaaactagggctaaactagaactagactagaactactcctaattgtgctgaaaataaatgcgagatagaagtggtattggttcgattgttgggggttcaatcggccgtatcccttcatctatataaagggggaggtctggatccgcttccaactgatttccgagttaatcccgcggttttaggtaacaaatcccgcgagaaactaggaaccctaactgactctgcgcacgcgcggaccgtccgcgccaccaccgcggacggtccggaccgcggaccgtccggcctccgggccggaccgtccgcacggtcattttgggttccaacatatgccccctgccttttggtgaaggtcgacaaaccaaaagcattgaactaaacctgatgtaagtcaccggcttttcgatatggagattattcaataaagcaccaatataaaggccgtttcggattgtatctttctcggccatgaccatttgatcaatggatcaaaaggaatagaatggaggtgccccccagtctggatagacgaagggactatacatgtaccatggattcatcatcgtgccattccatgtttgaacaggataatataccgacgatgagtaaataggtggaaagtaccctggtctcatagaatgaataggcgatgcttgttgtgtcgcctttcgggccgtctttgtttaaccgttttgttttagcaggtggctggggtttctttgttgaccgatcacgtggaacagtctttttgctagcatttttggagagcaactgatcaaaagtaggatcggctttgatcagccgattatatgtgctttgaccttgcgcctttttccttgctttgtgtagaggttgacgcctttggtcatagggggactgtccggctgagttagccggaccgtttgtctgagcaccggatcgtccgcacgtaggtgccggaccatctacgatgttcgggctaggctgatgtgtttggttcattaactgtgcctgccccccagtgtcttcggactttttagccttctcgtccgaagcctttcgagcaatctctttttgtgatatatctgacatgcggggatcgccaatgacgatacccttgcctttgcctttatcggccatttcgggccgaaccaagacctttttgcatgtgagttctattgtattaacaggaaagggttgtgtgtctacttgcatctcctgaaaagccaatcgacactcatttatggccgattgtatctgtcgacgaaaaacattacaatcattggtggcatgagaaaaggaattatgccacttacaataagcacgcctctttaattcatcaggaggaggaatagtatgagttaatttaatgttgccgtttttcagtaactcgtcaaatattttatcgcatttggcaacattaaacgtaaacttaacttcttcttgtcgattctttcgaatcgactgtaaagcagaacacggtgaaggtttagcctgccctggccaaaccagttcagcggtgtatacatccgtggattcatcgtccgagttatcatatcccactagatgcatcttatggctagccgattttgatgtttccttacttcggctttcacatgtcatagcccgctggtgcaagtgcactagcgaaaagaattgggtaccatctaatttttcttttaagtagggtcgcaacccattgaaagctagccctgttagttgtttttccgcgacatgaatctgaaagcatcggtttctagtgtcccggaatctccggatatagtcattaaccgattcttcaggcccctgtcggactgaggctaagtcagccaattctaattcatgttctcctgagaagaagtgttcatgaaatttctgctctaattcttcccaagagttaatagagtttggtggcaaagttgcgtaccatgcaaatgcagtatcagtaagggacaacgaaaataaacgaacgcggtaggcttccccatcagccaattctcctaagtgtgctatgaattggctaatatgttcgtgtgtgcttttcccaccttcaccagaaaacttagagaagtctggtattctagttccctgtggatatggcacggtgtcgaatcggtggctataaggcttccgatacgattgccctgtacctgacaaactaacaccgagtttgtccctgaacatcccggctacctcgtctctgatcctctccgccatatctggcgaccatctattgagtttgtgggtggaaacctcaggttgcctgacatcactctgtcggctttcctcccagggatgtttgaggtgtgcgctaggtgtcctattaatgtcaccaactcctgccctatacctctcaggctctcttgtggccgaacagtattcagccctatgtccatgaggtggtatggcatgattataatgtgttactggtggggcaccataatgttgctgcgatgaatgcggaaactgtgcgtattgcgcagctctcggctctgcgtatgcatatccggacggtccggcataagaggccggatggtccgcgacctggccaaatggttcgaaggtatatccggattgtccagccgtatatggtgctacatgggtagtctcgtacccagaccgtctgtcgtagataactggacggtccgcgatcgggccgaatggtccagggctgtacccggacggatcggtcatatatggcgcgacttgggtagtctgcgcgtggactcgtgtagagtcggatggtccagcgtaatacgtcggccggttcatgccatatccggacggtccggcgtaagatggcgaacggttcgcaacatatccggacggtccggcgtgatgcaccggacggtccgtgatggggccgaagtgttcagggttgtaccctgatggtccggccatgtacggtgcgacctgagtgttttgcgtgcgggcctgcatctggtcggacggcccggcgaaatacgccggacggtccgcggtataaccggactgtctgagatgatgctcggacggtccggtcgcgtccagtacctgccgtgtgcctagtggttgcggctgtgatggtgacacgaaagcgtgcatcggcataccatatgatggctgggtcaagggtgacccgtttatagccgatgtgtttgacgtgggtggcactgtattagactctcgtgatggaaagttaggagcagctgatttctcgtatgcacgtaaatgcattttaatagattcatctacatattgctttaattgatctcctcgttgatccatgaaagtcgtaagagataggtctggagtacttacagcgggaccctgaagtggaggtaggagagattccatatcgatctccccttgacggacgatcttctggtggcgatctaccgtgaagtgtgacaagtacttgtctgccgcctccttgcgcctttcggagagtttatgcagtagttccgcctcttccttgtcgtgttgttcgttccattgccgcatcacctccttctcatcgcgcattacgaggtcttcaaagggcctttggtcatcagccgggagcgcctccatggccggcttgatgatgttggtagtggagatcttggtgtgatccttagaaccggccatttatgggccgatttttgcagattagacacctagtccccagcggagtcgccaaaaagtacgttgacacctttttggaggtgcaatcacttcaaaagaaccggcggcggtgctctctggtcaggcgcggacggtccgcggcacagggccggacggtccgcgacctggcgtgaggtggcggtgctctctggtcaggcgcggacggtccgcggcacagggccggacggtccgcgacctggcgtgaggtggcggtgctctctggtcaggcgcggacggtccgcggcacagggccggacggtccgcgacctggagcaggagctcgggttccctgcctgacggtcggacggtccgcgcgtgcgcaggggcggcggaagatcgccagcggcgcctggatctcgctcccgggagggaccccgtcggggaggagagatcctaggagttgtttaggctcgggccggccgacctagactcctttaatcgacgtagagtcgaggagaggcggagaatttggggattggaatactaaactagggctaaactagaactagactagaactactcctaattgtgctgaaaataaatgcgagatagaagtggtattggttcgattgttgggggttcaatcggccgtatcccttcatctatataaaggggaggtctggatctgcttccaactgatttccgagttaatccagcggttttaggtaacaaatcccgcgagaaactaggaaccctaactgactctgcgcacgcgcggaccgtccgcgccaccaccgcggacggtccggaccgcggaccgccgcacggtcattttgggttccaacacacggtataaacattagaaattggtacggattaaggctaagcgaacagcctagagactgtgagcgctcgaatcccaccttgtgggagcaccggagcacatgtgcagcttcgagccatactggacgctgcactgaaagttttggcattcatatagtaaacgtccgtggtcgacaggcacccacggccttgaacatagcgatggaagtcatggatcgacgaagctgattgagtcagttacaccgaagtcgattgacaaaggctatctaccacgacatcaaatcgcacaggaagacgtgatgaatagcaggtagagagagagggtaaaagatgtagcagattggttttttgatgattgaaagagtcgaccgtgttcatctgatatacgtagaggtggtggtcttatctgagcttccacatgctgcgatcgatttgttggtccccatcttgctctcccacacaggaatactattaaccatgttcaggcaagaaagtgatgcggtcgtgcacggcacatgccagctttgtgggagccgcccctaaccctcgctgaatcagtcagtagtgccaacttgctagagttttttttcttcttgttttggttcactcgacagatttttgtttggatgagatcgctgcaacattgttcttgatccacacttgcctgatcttaccgtctcgttcgtgttcgtgccagcaaccagcgaaaatg

SEQ ID NO: 5 - DNA, 2907 bp, Zea mays
caggtacccgcattagccttcctctattctggatgatcccccctgccagtgtttcatagattgttctgaatggattgatgaggaatcgaccggagtttcggttttgggttgctgagctcggcagcagggtgacagttcgccgtagcgggctcgtgaccggccgtcgccggcggggcgggtccggccgagctcgtgtcgtcgatccgtagcgttgggtctgggagaaagtaatgggatgcggccgaactcgccgtaccccccgccggtcgagcttgacatcgatctcaccggcgggcatccgcacaagccttgcgctgccgatgtggatttgcccagattaatcctggcaaagcgcgcttgtttcccatctcatcagatctgtaggattcagcgtggggtgccgatcagatattttgcccgtgcaatggatccatgatctctgccccctcctgcccactcgtttcgggaacatgacatgccacttttggccacgaacttttcgcagctcccgtcaatcttgtgggtaaaagctgcaacctttacaggcctagcctctttctttatgcgttcggtccctccatgacagccatcgctgcgcctgcgccctccccatgatggccaactgctccgttgttctatcttctgatttttttactggtactattagctaagcacggagttggcgacaattgcacccaagaattgactgaccttttagctccagcaattgctgtgtctaggaagcaactcgttctgctttggtcacacataaaaaatatctacttgtccagatgggaaaccgtatatgcttttctaggaatttggatagaaaaaaatagagcgcgttcctttcaatcccagtcatcacacgctcgaggtsgagggcaggaaaccgccggcggcggcggcggcagcggggatggggagctcgttccgtgggtcttgtctgcttgacctagaaaacggcatcgtgatgaasgacgcgctaccgtccgatgccttgggattttggacggtggcgactgtctcctcccasgtggccacgtacagtcaaaaaccgagacagaaaaagatttcacctactccgcctcaccttcggcatgggccggcggcatgtcagggctctgcagctgtgtctgcgcaacggtacaagacgccgcgggggtcgcagcctgcaaggccggcaccgaattctaggccccacatgatggcatgcaacaccggtgcacagatatttttcgacacgattatccagccgtagaataactcggacaagtgtcgagaggcgtggactagcagatctgggtgcagttggcccctctggtgaccagagtgacccgtccttcaccttggcgtggtcggctgcaactcgctgtccgatgcaaattgctgctactgctatgtccatggcatggagtcgcatgtgccatttcttccctgtttgtttggctctccccgccgtccgatcagaaagttagggagacaatttaggccctgttcctatctcgcgagataaactttagcagcttttttttagctacttttagccatttgtaatctaaacaggagagctaatggtggaaattgaaactaaactttagcacttcaattcatatagctaaagtttagcaggaagttaaagtttatcccgtgagattgaaacgggcctttagacgggcggcccttgtcttgtcagaattaatgcacagtatcggcacggcggccaagcatctctttcgacggatctggtttctgtctccatctgtgggcgccatggttggctggtcgacaggacgcgcttgtgtcatttgggccaagccccaagggagacagataacatccgattccacctcgtgcgagcacatgtgcggcttcgagccataccataccatactgaatgccgcacttccaaagttttggcatcactgataaacgcccaaattttggtaacaagatgaagcaaacagacaatgaaaaaccggatcttttctaagatttatactaatgcgccgtgcatcttttacgttgctatatggtgcttcactaggctttatcgtaaaccgaactgatttaccaccaccttcaatgcacaaggcagagcacctgccatcttacgctgatttttttttgaaatatggtgtgcctctaggctctggactggtaggtgggtttgcatgtagaaaagatgacttgggagctcatgcttgctagcttgtcaaaattgaccacttctaccgatgacgcaagattgccttgctctgtatggctattggatagcttagatttgaccatatatggtagtactaccatttatttttccttccgctgaatcacctcaacgcacgttcttggcgctgccgcttgttagtctctcctgcctgctgctttccattggtccagaagtccctttcacaaatcaccgtccaattgcatgcagtacatcacatgtttctcaagggggttgttggaccagttcgttcaatgtaacatcacaagcgacaggaccttaatctgttttctgcttatttaatgtagatttgccgtagggttttgtaccatccttggtcttgctgtaaagtctgcattttattagttctgtgtggtggtaatcagaattgctggtttgggctcgcacatgctgtgatccccaacttgctgtggcgtggtagttggatcgtgtttaggcaagaaagtaaatgcgatcatgcacggcatatttgccaccttcctgggagacgccccctcgtgccgtgatctgttttactttggttgattggtggcctttctcgtggttcacgtgacagcttttctgatgggatgagatcactgtaatgttgttgcttgattcacgctcgcttgatcttactgtagcgtacttcctcgtttgtgtcagtcaggagcaagatg

SEQ ID NO: 6 - DNA, 18 bp, Artificial Sequence
  Description of Artificial Sequence  SYNTHETIC OLIGONUCLEOTIDE
gayatgathacngargar

SEQ ID NO: 7 - DNA, 17 bp, Artificial Sequence
  Description of Artificial Sequence SYNTHETIC OLIGONUCLEOTIDE
ccrtcrtacatnagatg

SEQ ID NO: 8 - DNA, 1714 bp, Zea mays
  CDS (134)..(1312)
ggcacgagctcactgccatttgtttggttgttcctctcgctcgccagtcgccaccaggcagcaggcatcccaatctcgcgagagccagtagcggcggcggcgcttccggcttcccttcccattggcctccgggatggcgctccgcctccacgacgtcgcgctctgcctctccccgccgctcgccgcccgccgccgcagcggcggcagtttcgtcgccgtcgcctccatgacgtccgccgccgtctccaccagggtggagaacaagaagccatttgctcctccgagggaggtacatgtccaggttacacattcaatgccatctcacaagattgaaattttcaagtcacttgatgattgggctagagataatatcttgacacatctcaagccagtcgagaagtgttggcagccacaggatttcctccctgacccagcatctgaaggatttcatgatgaagttaaggagctcagagaacgtgccaaggagatccctgatgattattttgtttgtttggttggagacatgattactgaggaagctctaccaacataccagactatgcttaacaccctcgacggtgtcagagatgagacaggtgcaagccccactgcttgggctgtttggacgagggcatggactgctgaggagaacaggcatggtgatcttctcaacaagtacatgtacctcactgggagggtagatatcaggcaaattgagaagacaattcagtatcttattggctctggaatggatcctaggactgagaataatccttatcttggtttcgtctacacctccttccaagagcgggcgaccttcatctcgcatgggaacactgctcgtcatgccaaggactttggcgacttaaagctcgcacaaatctgtggcatcatcgcctcagatgagaagcgacatgaaactgcgtacaccaagatcgtggagaagttgtttgagatcgaccctgatggtacagtggttgctctggctgacatgatgaagaagaagatctcaatgcctgcccacctgatgtttgacggtcaggacgacaagctgtttgagcacttctccatggtcgcgcagaggcttggcgtttacaccgccagggactacgccgacattcttgagttccttgttgacaggtggaaggtggcggacctgactggtctgtcgggtgaggggaacaaggcgcaggactacctctgcacccttgcttcaaggatccggaggctagacgagagggcccagagcagagccaagaaagcaggcacgctgcctttcagctgggtatatggtagggaagtccaactgtgaaatcggaaacccattgcgactgcttgagttggagcatagtctatcatgcaccctatgacgcatcgcacgacaagacctggtgtgtcgcgtgacatagttgttcaggttttgaccaaatggtctgggagcatttgttttgccttgtgccgtctcatagagcgttaggatagtgtacgtctgtgttctagctttgttttgtctgctgctttgatgtaacttgtggccatgaggctggacatggagtgaacatgttgtacattgtcgctggcggtatgtttcggtatgttatttcagttgcttgagatctgttaattttttgcgcagctatggaggtcgttctgttctggtcaaaaaaaaaaaaaaaaaaaaaaaaaaaaaaaaaaaaaaaaaaaaaaaaaaaaa

SEQ ID NO: 9 - protein, 392 aa, Zea mays
MALRLHDVALCLSPPLAARRRSGGSFVAVASMTSAAVSTRVENKKPFAPPREVHVQVTHSMPSHKIEIFKSLDDWARDNILTHLKPVEKCWQPQDFLPDPASEGFHDEVKELRERAKEIPDDYFVCLVGDMITEEALPTYQTMLNTLDGVRDETGASPTAWAVWTRAWTAEENRHGDLLNKYMYLTGRVDIRQIEKTIQYLIGSGMDPRTENNPYLGFVYTSFQERATFISHGNTARHAKDFGDLKLAQICGIIASDEKRHETAYTKIVEKLFEIDPDGTVVALADMMKKKISMPAHLMFDGQDDKLFEHFSMVAQRLGVYTARDYADILEFLVDRWKVADLTGLSGEGNKAQDYLCTLASRIRRLDERAQSRAKKAGTLPFSWVYGREVQL

SEQ ID NO: 10 - DNA, 1709 bp, Zea mays
  CDS (102)..(1280)
cggcacgagcacacacaagggaaggggacaaccacaagcgcctaagatcccgtcctccgcgtcgagatctttgccgaggcggtgaccgtcgagggatcgccatggcgttgagggcgtcccccgtgtcgcatggcaccgcggcagcgccgctgccgcctttcgcgcggaggaagatggcccgtggggtggtggtggccatggcgtccaccatcaacagggtcaaaactgtcaaagaaccctatacccctccacgagaggtacatcgccaaattacccattcactaccacctcaaaagcgggagattttcgattcacttcaaccttgggccaaggataacctactgaacctactgaagccagttgaaaagtcatggcagccacaggacttcctaccagagccttcttctgatgggttttatgatgaagttaaagaactgagggagcgggcaaatgaaatacctgatgaatactttgtttgcttagttggtgatatggttactgaggaagccttacctacataccaaacaatgcttaacactcttgatggagtccgggatgaaactggtgcaagttcaaccacgtgggcggtttggacaagggcatggacagctgaagagaacagacatggtgacctccttaacaagtacatgtaccttactggacgggttgacatgaaacaaattgagaagaccatacaatatctgattggttccggaatggatcctggaactgagaacaacccctacttgggtttcctctacacatcattccaagaaagggcaacatttgtgtcgcatgggaatactgcaaggcatgccaaggagtatggtgatctcaagctggcccagatatgtggcacgatagcagccgatgagaagcgccacgaaacagcctacaccaagatagtcgagaagctcttcgagatggaccctgattacacagtgcttgcgtttgctgacatgatgaggaagaagatcacgatgccagcccatctcatgtacgacggtaaggacgacaacctgttcgagcacttcagcgcggtggcgcagaggctgggcgtctacaccgccaaagactacgccgacatcctcgagttcctggtccagaggtggaaagtcgcggagctcacagggctgtctggagaagggagaagcgcgcaggactttgtctgtaccttggcgccgaggatcaggcggctggatgatagagctcaagcgagggcgaagcaagcaccggttattcctttcagttgggtttatgaccgcaaggtgcagctttaatcaagaacgctaggcaatgtgggcatttactacgtatatcattttcagtcctggggttctctataagaaacagtctctaggttatctagcagggtagaattcaactactcgtggatctcactcggtgcaaagtagtgcaaagtacgctatctgttgttaccgtgcaagctgcagagtttggattactatgtgggcctggtggtggagaggaattctgtggggtgcctgcagccagttatgagtggcagctccatcgcaactgagttgttgtattgaatatgttacaggacctatagtaaccgaaagtaataatatggagtttgtatatcgacaagcttgctttggtgattgatgagaatctgaagtaataatatggagtttgcataaaaaaaaaaaaaaaaaaaaaaaaaaaaaaaaaaaaaaaaaaaa

SEQ ID NO: 11 - protein, 392 aa, Zea mays
MALRASPVSHGTAAAPLPPFARRKMARGVVVAMASTINRVKTVKEPYTPPREVHRQITHSLPPQKREIFDSLQPWAKDNLLNLLKPVEKSWQPQDFLPEPSSDGFYDEVKELRERANEIPDEYFVCLVGDMVTEEALPTYQTMLNTLDGVRDETGASSTTWAVWTRAWTAEENRHGDLLNKYMYLTGRVDMKQIEKTIQYLIGSGMDPGTENNPYLGFLYTSFQERATFVSHGNTARHAKEYGDLKLAQICGTIAADEKRHETAYTKIVEKLFEMDPDYTVLAFADMMRKKITMPAHLMYDGKDDNLFEHFSAVAQRLGVYTAKDYADILEFLVQRWKVAELTGLSGEGRSAQDFVCTLAPRIRRLDDRAQARAKQAPVIPFSWVYDRKVQL

SEQ ID NO: 12 - DNA, 18 bp, Artificial Sequence
  Description of Artificial Sequence  SYNTHETIC OLIGONUCLEOTIDE
aggacgctaccgtaggaa

SEQ ID NO: 13 - DNA, 17 bp, Artificial Sequence
  Description of Artificial Sequence  SYNTHETIC OLIGONUCLEOTIDE
gcgatggcactgcagta

SEQ ID NO: 14 - DNA, 21 bp, Artificial Sequence
  Description of Artificial Sequence  SYNTHETIC OLIGONUCLEOTIDE
cttgagagaagaaccacactc

SEQ ID NO: 15 - DNA, 21 bp, Artificial Sequence
  Description of Artificial Sequence  SYNTHETIC OLIGONUCLEOTIDE
ctagacatatcgagcatgctg

SEQ ID NO: 16 - DNA, 22 bp, Artificial Sequence
  Description of Artificial Sequence  SYNTHETIC OLIGONUCLEOTIDE
aggcgctgacggtggcgacgct

SEQ ID NO: 17 - DNA, 20 bp, Artificial Sequence
  Description of Artificial Sequence  SYNTHETIC OLIGONUCLEOTIDE
gtgttggcgaggcacgtgag

SEQ ID NO: 18 - DNA, 46 bp, Artificial Sequence
  Description of Artificial Sequence  SYNTHETIC OLIGONUCLEOTIDE
acctcccgtcgcaccccggtggtgatcagccatggtaggctagcag

SEQ ID NO: 19 - DNA, 1714 bp, Artificial Sequence
  Description of Artificial Sequence  SYNTHETIC OLIGONUCLEOTIDE
tctagaagtgtatgtatgtcaaagatcttatcgggataagagatatgataaagatcttaacggaatcagagccaggtttgtaaaaatagagttggactcgtgtacaacttggtctctggcttagctccgtcatgaatttagtaaccgactcgatatgtaccgtggaacccctagggcatgagccataggatcatcatatccaaacatgcaccaacaaatccaccacacatcgaagatccatattaagaaggggttatctactttacaatttcagagtaaccaatagagccaaactcatagcacaggggagcttcatatcagatggagccattgaattgatataaaaagctgaagttctaaaaagttttaagtgctggaacttcaaagccgctaactagtgaagcaccgaagccttccgtggagagatacatacacgacacgttagggacgtaaaatgacggaattatacagctacctctatatgtgacacttatgtaatagaaaagacagaatccatatgaagatgtataatggatcaatcatataaatagataaacaattgaggtgtttggtttgatgaatcactctatccaaaataaagtggtgcatcatgggtttattcctcaaatttggtggcatgactacattccacatattagtactaagcaactaactttgaggaatgaggtgatgatgaattaactcactccattccacaaaccaaacaaaaatttgaggagtgagaagatgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctaccatggtacgtggcat

SEQ ID NO: 20 - DNA, 32 bp, Artificial Sequence
  Description of Artificial Sequence  SYNTHETIC OLIGONUCLEOTIDE
cttatgtaatagaaaagacaggatccatatgg

SEQ ID NO: 21 - DNA, 33 bp, Artificial Sequence
  Description of Artificial Sequence  SYNTHETIC OLIGONUCLEOTIDE
gaggagtgaggatcctgattgactatctcattc

SEQ ID NO: 22 - DNA, 33 bp, Artificial Sequence
  Description of Artificial Sequence  SYNTHETIC OLIGONUCLEOTIDE
tctggacaccctaccattggatcctcttcggag

SEQ ID NO: 23 - DNA, 32 bp, Artificial Sequence
  Description of Artificial Sequence  SYNTHETIC OLIGONUCLEOTIDE
agagttggatccgtgtacaacttggtctctgg

SEQ ID NO: 24 - DNA, 37 bp, Artificial Sequence
  Description of Artificial Sequence  SYNTHETIC OLIGONUCLEOTIDE
gccgctgatgctcgagctacgactacgagtgaggtag

SEQ ID NO: 25 - DNA, 32 bp, Artificial Sequence
  Description of Artificial Sequence  SYNTHETIC OLIGONUCLEOTIDE
atgcgggactcgagtcgggggcagcgcgacac

SEQ ID NO: 26 - DNA, 32 bp, Artificial Sequence
  Description of Artificial Sequence  SYNTHETIC OLIGONUCLEOTIDE
gtggcggggccgaatctcgagtgggccgtagt

SEQ ID NO: 27 - DNA, 33 bp, Artificial Sequence
  Description of Artificial Sequence  SYNTHETIC OLIGONUCLEOTIDE
gccacgtgccatggtaggctagcagagcgagct

SEQ ID NO: 28 - DNA, 24 bp, Artificial Sequence
  Description of Artificial Sequence  SYNTHETIC OLIGONUCLEOTIDE
aacacacacccatggatatcacag

SEQ ID NO: 29 - DNA, 19 bp, Artificial Sequence
  Description of Artificial Sequence  SYNTHETIC OLIGONUCLEOTIDE
ggtctgacttacgggtgtc

SEQ ID NO: 30 - DNA, 25 bp, Artificial Sequence
  Description of Artificial Sequence  SYNTHETIC OLIGONUCLEOTIDE
ctctcccgtcctcgagaaaccctcc

SEQ ID NO: 31 - DNA, 25 bp, Artificial Sequence
  Description of Artificial Sequence  SYNTHETIC OLIGONUCLEOTIDE
cttggcagccatggctcgatggttc

SEQ ID NO: 32 - DNA, 30 bp, Artificial Sequence
  Description of Artificial Sequence  SYNTHETIC OLIGONUCLEOTIDE
atggtgagcgccagaatcgttgtcctcctc

SEQ ID NO: 33 - DNA, 30 bp, Artificial Sequence
  Description of Artificial Sequence  SYNTHETIC OLIGONUCLEOTIDE
catcctggcggtcaccatcctcaggagcgt

SEQ ID NO: 34 - DNA, 30 bp, Artificial Sequence
  Description of Artificial Sequence  SYNTHETIC OLIGONUCLEOTIDE
atagggaattctctgtttttctaaaaaaaa

SEQ ID NO: 35 - DNA, 30 bp, Artificial Sequence
  Description of Artificial Sequence  SYNTHETIC OLIGONUCLEOTIDE
gctcaccatggtgtagtgtctgtcactgtg

SEQ ID NO: 36 - DNA, 36 bp, Artificial Sequence
  Description of Artificial Sequence  SYNTHETIC OLIGONUCLEOTIDE
gggggatccaagcttgaggagacaggagataaaagt

SEQ ID NO: 37 - DNA, 39 bp, Artificial Sequence
  Description of Artificial Sequence  SYNTHETIC OLIGONUCLEOTIDE
gggctgcagctcgagggtgtagtgtctgtcactgtgata

SEQ ID NO: 38 - DNA, 1108 bp, Artificial Sequence
  Description of Artificial Sequence  SYNTHETIC OLIGONUCLEOTIDE
atccatatgaagatgtataatggatcaatcatataaatagataaacaattgaggtgtttggtttgatgaatcactctatccaaaataaagtggtgcatcatgggtttattcctcaaatttggtggcatgactacattccacatattagtactaagcaactaactttgaggaatgaggtgatgatgaattaactcactccattccacaaaccaaacaaaaatttgaggagtgagaagatgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctc

SEQ ID NO: 39 - DNA, 871 bp, Zea mays
tgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctc

SEQ ID NO: 40 - DNA, 545 bp, Zea mays
atcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctc

SEQ ID NO: 41 - DNA, 952 bp, Zea mays
tgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctacc

SEQ ID NO: 42 - DNA, 1403 bp, Zea mays
cgtgtacaacttggtctctggcttagctccgtcatgaatttagtaaccgactcgatatgtaccgtggaacccctagggcatgagccataggatcatcatatccaaacatgcaccaacaaatccaccacacatcgaagatccatattaagaaggggttatctactttacaatttcagagtaaccaatagagccaaactcatagcacaggggagcttcatatcagatggagccattgaattgatataaaaagctgaagttctaaaaagttttaagtgctggaacttcaaagccgctaactagtgaagcaccgaagccttccgtggagagatacatacacgacacgttagggacgtaaaatgacggaattatacagctacctctatatgtgacacttatgtaatagaaaagacagaatccatatgaagatgtataatggatcaatcatataaatagataaacaattgaggtgtttggtttgatgaatcactctatccaaaataaagtggtgcatcatgggtttattcctcaaatttggtggcatgactacattccacatattagtactaagcaactaactttgaggaatgaggtgatgatgaattaactcactccattccacaaaccaaacaaaaatttgaggagtgagaagatgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgac

SEQ ID NO: 43 - DNA, 990 bp, Zea mays
atccatatgaagatgtataatggatcaatcatataaatagataaacaattgaggtgtttggtttgatgaatcactctatccaaaataaagtggtgcatcatgggtttattcctcaaatttggtggcatgactacattccacatattagtactaagcaactaactttgaggaatgaggtgatgatgaattaactcactccattccacaaaccaaacaaaaatttgaggagtgagaagatgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgac

SEQ ID NO: 44 - DNA, 753 bp, Zea mays
tgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgac

SEQ ID NO: 45 - DNA, 427 bp, Zea mays
atcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgac

SEQ ID NO: 46 - DNA, 1248 bp, Zea mays
cgtgtacaacttggtctctggcttagctccgtcatgaatttagtaaccgactcgatatgtaccgtggaacccctagggcatgagccataggatcatcatatccaaacatgcaccaacaaatccaccacacatcgaagatccatattaagaaggggttatctactttacaatttcagagtaaccaatagagccaaactcatagcacaggggagcttcatatcagatggagccattgaattgatataaaaagctgaagttctaaaaagttttaagtgctggaacttcaaagccgctaactagtgaagcaccgaagccttccgtggagagatacatacacgacacgttagggacgtaaaatgacggaattatacagctacctctatatgtgacacttatgtaatagaaaagacagaatccatatgaagatgtataatggatcaatcatataaatagataaacaattgaggtgtttggtttgatgaatcactctatccaaaataaagtggtgcatcatgggtttattcctcaaatttggtggcatgactacattccacatattagtactaagcaactaactttgaggaatgaggtgatgatgaattaactcactccattccacaaaccaaacaaaaatttgaggagtgagaagatgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccac

SEQ ID NO: 47 - DNA, 835 bp, Zea mays
atccatatgaagatgtataatggatcaatcatataaatagataaacaattgaggtgtttggtttgatgaatcactctatccaaaataaagtggtgcatcatgggtttattcctcaaatttggtggcatgactacattccacatattagtactaagcaactaactttgaggaatgaggtgatgatgaattaactcactccattccacaaaccaaacaaaaatttgaggagtgagaagatgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccac

SEQ ID NO: 48 - DNA, 598 bp, Zea mays
tgattgactatctcattcctcaaaccaaacacctcaaatatatctgctatcgggattggcattcctgtatccctacgcccgtgtaccccctgtttagagaacctcccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccac

SEQ ID NO: 49 - DNA, 272 bp, Zea mays
atcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccac

SEQ ID NO: 50 - DNA, 29 bp, Artificial Sequence
  Description of Artificial Sequence  SYNTHETIC OLIGONUCLEOTIDE
cggggtaccgatgaccgagaaggagcggg

SEQ ID NO: 51 - DNA, 29 bp, Artificial Sequence
  Description of Artificial Sequence  SYNTHETIC OLIGONUCLEOTIDE
ggcggtacctagaacttcttgttgtacca

SEQ ID NO: 52 - DNA, 31 bp, Artificial Sequence
  Description of Artificial Sequence  SYNTHETIC OLIGONUCLEOTIDE
ggcctccgccatggcgctccgctccacgacg

SEQ ID NO: 53 - DNA, 30 bp, Artificial Sequence
  Description of Artificial Sequence  SYNTHETIC OLIGONUCLEOTIDE
ctccaactcaagcagtcgccatgggtttcc

SEQ ID NO: 54 - DNA, 20 bp, Artificial Sequence
  Description of Artificial Sequence  SYNTHETIC OLIGONUCLEOTIDE
ctgcactgaaagttttggca

SEQ ID NO: 55 - DNA, 25 bp, Artificial Sequence
  Description of Artificial Sequence  SYNTHETIC OLIGONUCLEOTIDE
agtacagcggccaggcggcgtagcg

SEQ ID NO: 56 - DNA, 20 bp, Artificial Sequence
  Description of Artificial Sequence  SYNTHETIC OLIGONUCLEOTIDE
aaggggagagagaggtgagg

SEQ ID NO: 57 - DNA, 20 bp, Artificial Sequence
  Description of Artificial Sequence  SYNTHETIC OLIGONUCLEOTIDE
tgcattgaaggtggtggtaa

SEQ ID NO: 58 - DNA, 6337 bp, Zea mays
gtcgactctagaggatccgattgactatctcattcctccaaacccaaacacctcaaatatatctgctatcgggattggcattcctgtatccctacgcccgtgtaccccctgtttagagaacctcccaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacccaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggaagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcgagaaaccctccctccctcctccattggactgcttgctccctgttgaccattggggtatgcttgctctcctgttcatctccgtgctaaacctctgtcctctgggtgggtttttgctgggattttgagctaatctgctggccgcggtagaaaagaccgtgtcccctgatgagctcaagcgctcgccttagccgcgtccttgtcccccgccatttcttgcggtttcgctgtgttcccgtgactcgccgggtgcgtcatcgcctgaatcttgtctgggctctgctgacatgttcttggctagttgggtttatagattcctctgatctaaaaccgtgcctgtgctgcgcacagaactctcccctgtcctttcctggggttttggttacgtggtggtagtaagcttggatttgcacatggataaagttgttctaagctccgtggtttgcttgagatcttgctgttattgcgtgccgtgctcacttcttttgcaatccgaggaatgaatttgtcgtttactcgttttggtggattattagcgcgaaaaaaaactctttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactattgagacaaaatcaatccaatcaagttgatttctttctttaaaaatattatcacagaactaagtgcttgtgcggaatcagtactggcttttgtttggtggaggatcaatacttgcttttgttttggggtggcaactgttttgctataagattccatgtgttcctgttgagatgaatcatatatagtatagctgcatactacaaatctgtttttcaaatttaggttgctttggcatgatcaattttttttcagacagtctttctaagtggtagctcttgatttcttgttcttctacaactggtgctgctgaatcttgaccgtatagctcgaattgcagtattctgaaccatcgagccatgaattcccccgatgaccgagaaggagcgggagaagcaggagcagctcgcccgagctaccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactactcgctcctggacgacgtggtcggcctggtgctgcactcgtcgctcatggtgccctacttctcgtggaagtacagccaccggcgccaccactccaacacggggtccctggagcgcgacgaggtgttcgtgcccaagaagaaggaggcgctgccgtggtacaccccgtacgtgtacaacaacccggtcggccgggtggtgcacatcgtggtgcagctcaccctcgggtggccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtacaagctggcggcggcgttcggggtctggtgggtggtgcgcgtgtacgccgtgccgctgctgatcgtgaacgcgtggctggtgctcatcacctacctgcagcacacccacccgtcgctcccccactacgactcgagcgagtgggactggctgcgcggcgcgctggccaccatggaccgcgactacggcatcctcaaccgcgtgttccacaacatcacggacacgcacgtcgcgcaccacctcttctccaccatgccgcactaccacgccatggaggccaccaaggcgatcaggcccatcctcggcgactactaccacttcgacccgacccctgtcgccaaggcgacctggcgcgaggccggggaatgcatctacgtcgagcccgaggaccgcaagggcgtcttctggtacaacaagaagttctaggggggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgactctagaaagcttactagtgatgcatattctatagtgtcacctaaatctgcggccgctgaccaagtcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggatgccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttggccaagtcggcctctaatacgactcactatagggagctc

SEQ ID NO: 59 - DNA, 1146 bp, Zea mays
atgaccgagaaggagcgggagaagcaggagcagctcgcccgagctaccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactactcgctcctggacgacgtggtcggcctggtgctgcactcgtcgctcatggtgccctacttctcgtggaagtacagccaccggcgccaccactccaacacggggtccctggagcgcgacgaggtgttcgtgcccaagaagaaggaggcgctgccgtggtacaccccgtacgtgtacaacaacccggtcggccgggtggtgcacatcgtggtgcagctcaccctcgggtggccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtacaagctggcggcggcgttcggggtctggtgggtggtgcgcgtgtacgccgtgccgctgctgatcgtgaacgcgtggctggtgctcatcacctacctgcagcacacccacccgtcgctcccccactacgactcgagcgagtgggactggctgcgcggcgcgctggccaccatggaccgcgactacggcatcctcaaccgcgtgttccacaacatcacggacacgcacgtcgcgcaccacctcttctccaccatgccgcactaccacgccatggaggccaccaaggcgatcaggcccatcctcggcgactactaccacttcgacccgacccctgtcgccaaggcgacctggcgcgaggccggggaatgcatctacgtcgagcccgaggaccgcaagggcgtcttctggtacaacaagaagttctag